(19) United States
(12) Patent Application Publication
(10) Pub. No.: US 2013/0122095 A1
(43) Pub. Date: May 16, 2013

(54) PLATELET-DERIVED GROWTH FACTOR COMPOSITIONS AND METHODS FOR THE TREATMENT OF OSTEOCHONDRAL DEFECTS

(71) Applicant: BIOMIMETIC THERAPEUTICS, INC., Franklin, TN (US)

(72) Inventors: Hans K. Kestler, Brentwood, TN (US); Joshua Nickols, Nashville, TN (US); Leslie A. Wisner-Lynch, Franklin, TN (US); Colleen M. Roden, Murfreesboro, TN (US); Yanchun Liu, Franklin, TN (US)

(73) Assignee: BIOMIMETIC THERAPEUTICS, INC., Franklin, TN (US)

(21) Appl. No.: 13/667,566

(22) Filed: Nov. 2, 2012

Related U.S. Application Data
(63) Continuation of application No. 12/718,942, filed on Mar. 5, 2010, now abandoned.
Application No. 12/718,942 is a continuation-in-part of application No. PCT/US2010/026454, filed on Mar. 5, 2010.
(60) Provisional application No. 61/209,520, filed on Mar. 5, 2009.
(60) Provisional application No. 61/164,259, filed on Mar. 27, 2009.

(51) Int. Cl. A61K 9/00

(52) U.S. Cl. CPC A61K 9/0002
USPC 424/484; 514/8.2

(57) ABSTRACT

The present invention provides compositions and methods for treating an osteochondral defect. In one embodiment, provided is a composition for treating an osteochondral defect comprising a biphasic biocompatible matrix and platelet derived growth factor (PDGF), wherein the biphasic biocompatible matrix comprises a scaffolding material and wherein the scaffolding material forms a porous structure comprising an osseous phase and a cartilage phase. In another embodiment, also provided is a method for treating an osteochondral defect in an individual comprising administering to the individual an effective amount of a composition comprising a biphasic biocompatible matrix and PDGF to at least one site of the osteochondral defect, wherein the biphasic biocompatible matrix comprises a scaffolding material and wherein the scaffolding material forms a porous structure comprising an osseous phase and a cartilage phase.

### CROSS-REFERENCE TO RELATED APPLICATIONS

This application claims the benefit under 35 U.S.C. §119(e) of U.S. Provisional Patent Application No. 61/209,520, filed Mar. 5, 2009, and U.S. Provisional Patent Application No. 61/164,259, filed Mar. 27, 2009, this application is also a continuation-in-part of PCT Application No. ______, filed on Mar. 5, 2010, Attorney Docket No. 597792001540, titled “Platelet-Derived Growth Factor Compositions And Methods For The Treatment Of Osteochondral Defects”; the entireties of which are herein incorporated by reference.

### TECHNICAL FIELD

This invention relates to compositions and methods for treating an injury or a defect in a cartilage and a bone, particularly to the treatment of osteochondral defects in a cartilage and a bone adjacent to the cartilage in an individual by administering compositions to the individual comprising a biphasic biocompatible matrix in combination with platelet-derived growth factor (PDGF) to at least one site of the osteochondral defect.

### BACKGROUND OF THE INVENTION

Cartilage is a specialized connective tissue composed of chondrocytes. In general, there are three main types of cartilage, namely articular (hyaline) cartilage, fibrocartilage, and elastic cartilage, all of which differ in structure and function.

Articular cartilage comprises a network of collagen fibers (Type II collagen) and a proteoglycan matrix containing chondrocytes. Its principle functions are to provide an almost frictionless articulating surface as well as to provide a shock-absorbent structure which can withstand compression, tension, and shear forces, and to dissipate load. The composition of articular cartilage varies with anatomical location on the joint surface, with age and with depth from the surface. See Lipshitz H. et al., J. Bone Joint Surg., 57(4):527-34 (1975). Articular cartilage differs from other musculoskeletal tissues in that it does not have the ability to regenerate following traumatic or pathologic challenges. Once disease or trauma affects the health of articular cartilage, an inevitable degenerative process can occur. See Convery F. R. et al., Clin. Orthop., 82:253-62 (1972).

Fibrocartilage is characterized by a dense network of Type I collagen. It contains more collagen and less proteoglycan than articular cartilage. It is present in areas most subject to frequent stress, such as intervertebral discs, meniscus, the symphysis pubis, and the attachments of certain tendons and ligaments.

Elastic cartilage contains large amounts of elastin throughout the matrix. It functions to prevent tubular structures from collapsing and can be found in the pinna of the ear and in tubular structures, such as auditory tubes and epiglottis.

Injury or trauma to the cartilage has been increasingly recognized as a cause of pain and functional problems in patients. Cartilage in general has limited repair capabilities because chondrocytes are bound in lacunae and cannot migrate to damaged areas. Further, in the case of articular cartilage damage, due to the absence of innervation and penetration by the vascular and lymphatic system and derivation of nutrition primarily through the synovial fluid and to some degree from the adjacent bone, injury or trauma to the articular cartilage is very difficult to heal, especially in the case of adult articular cartilage, which is mostly avascular and only 5% cellular. See Bora F. W. Jr. and Miller G., Hand Clin., 3(3):325-36 (1987).

There are two types of injury or defect recognized in the cartilage: chondral defects (or superficial defects) and osteochondral defects (or full-thickness defects). While injury or trauma in chondral defects is only restricted in the cartilage itself without affecting the subchondral bone structures, injury or trauma in osteochondral defects affects both the cartilage and its underlying bone, and is very difficult to treat. Osteochondral defects (or focal osteochondral defects) are believed to arise as traumatic injuries sustained at the cartilage surface that initiate a cascade of cell death in cartilage that transmits to bone, for example, as found in cases of severe osteoarthritis. The compressive forces further impact underlying bone and cause injury to the blood supply and eventual necrosis. Current treatments for osteochondral defects include osteoarticular transfer system (OATS)/mosaicplasty, allograft, autologous chondrocyte implantation (ACI)/Matrix-ACI (MACI), and microfracture. However, each of these treatments has various drawbacks.

Osteoarticular transfer system (OATS)/mosaicplasty requires transfer of cylindrical plugs of non-weight bearing healthy cartilage into areas of the damaged cartilage. This treatment is complicated by the technical challenges of optimal plug positioning and tissue necrosis from the force required for harvesting the tissues. Furthermore, patients often suffer from comorbidity of the harvest site and must remain in surgery for longer periods of time.

The second treatment option, allograft, is routinely used in knee procedures. However, it has the main drawbacks of disease transmission risk and inferior result in comparison to the fresh autologous tissue grafting.

Autologous chondrocyte implantation (ACI)/Matrix-ACI (MACI) requires a cartilage explant (between 200 mg and 300 mg) removed from a non-weight-bearing area in the knee (e.g., the femoral condyle). The chondrocytes in the tissue samples are then separated from their surrounding cartilage and cultured for four to five weeks. The defect area is prepared by removing dead cartilage and smoothing the surrounding living cartilage below. A piece of periosteum, the membrane which covers bone, is taken from the patient's tibia and sutured over the prepared defect, underneath which the cultured chondrocytes are injected by the surgeon. ACI has not been widely used due to its high cost (i.e., greater than $20,000 per procedure), necessity of two operations to harvest and implant the chondrocytes, increased operation time, localized morbidity at the harvest site, and inability to produce better outcomes than microfracture alone.

Microfracture surgery is performed through an arthroscopic approach. The surgeon first removes any calcified cartilage from the lesion with a curette or burr. Tiny fractures are then created in the adjacent bones through the use of an awl. Blood and bone marrow (which contains stem cells) seep out of the fractures, creating a blood clot that releases cartilage-building cells. The microfractures are treated as an injury by the body, and the surgery results in newly replaced cartilage. The procedure is less effective in treating older or overweight patients, or cartilage damage that is larger than 2.5 cm. Approximately 120,000 microfracture procedures (including Grades 3 and 4 lesions) occur per year. Microfracture is also an incomplete fix for the osteochondral injury, because 1) an insufficient clot and quantity of cells are drawn into the defect to regenerate cartilage; 2) delamination/migration of the clot occurs after formation; and 3) Type I collagen found in fibrocartilage is generated, not the desirable Type II hyaline cartilage.

Accordingly, there is a need to provide new compositions and methods for a more effective, efficient, and economical treatment for osteochondral defects in the cartilage and its underlying bone, in particular in the articular cartilage, fibrocartilage, or elastic cartilage, and its underlying bone.

All references cited herein, including, without limitation, patents, patent applications and scientific references, are hereby incorporated in their entirety.

### SUMMARY OF THE INVENTION

The present invention provides for compositions and methods for treating an osteochondral defect. In one aspect of the invention, a composition is provided comprising a biphasic biocompatible matrix and platelet derived growth factor (PDGF), wherein the biphasic biocompatible matrix comprises a scaffolding material and wherein the scaffolding material forms a porous structure comprising an osseous phase and a cartilage phase.

In another aspect of the invention, provided are methods for treating an osteochondral defect in an individual comprising administering to said individual an effective amount of a composition comprising a biphasic biocompatible matrix and platelet derived growth factor (PDGF) to at least one site of an osteochondral defect, wherein the biphasic biocompatible matrix comprises a scaffolding material and wherein the scaffolding material forms a porous structure comprising an osseous phase and a cartilage phase.

In some embodiments of the present invention, the osteochondral defect is in a cartilage and a bone adjacent to the cartilage, and the cartilage comprises an articular cartilage, a fibrocartilage, or an elastic cartilage.

In some embodiments of the present invention, the osteochondral defect is in a cartilage and a bone adjacent to the cartilage, and the bone adjacent to the cartilage comprises a subchondral bone or a cancellous bone.

In some embodiments, the at least one site of the osteochondral defect comprises the bone adjacent to the cartilage, the cartilage, an interface between the cartilage and the bone adjacent to the cartilage, or combinations thereof.

In some embodiments of the present invention, the osseous phase comprises a calcium phosphate and collagen. In some embodiments, the calcium phosphate is tricalcium phosphate. In some embodiments, the osseous phase comprises a calcium sulfate and collagen.

In some embodiments, the calcium phosphate consists of particles in a range of about 100 μm to about 5000 μm in size. In some embodiments, the calcium phosphate consists of particles in a range of about 100 μm to about 3000 μm in size. In some embodiments, the calcium phosphate consists of particles in a range of about 250 μm to about 1000 μm in size.

In some embodiments, the calcium phosphate used in the osseous phase has a lower volume percentage in comparison to the total of volume of the biphasic biocompatible matrix. In some embodiments, the calcium phosphate has a volume percentage ranging from about less than about 5% to about less than 50% of the total volume of the biphasic biocompatible matrix. In some embodiments, the calcium phosphate has a volume percentage less than about 5% of the total volume of the biphasic biocompatible matrix. In some embodiments, the calcium phosphate has a volume percentage less than about 10% of the total volume of the biphasic biocompatible matrix. In some embodiments, the calcium phosphate has a volume percentage less than about 15% of the total volume of the biphasic biocompatible matrix. In some embodiments, the calcium phosphate has a volume percentage less than about 20% of the total volume of the biphasic biocompatible matrix. In some embodiments, the calcium phosphate has a volume percentage less than about 30% of the total volume of the biphasic biocompatible matrix. In some embodiments, the calcium phosphate has a volume percentage less than about 35% of the total volume of the biphasic biocompatible matrix. In some embodiments, the calcium phosphate has a volume percentage less than about 40% of the total volume of the biphasic biocompatible matrix. In some embodiments, the calcium phosphate has a volume percentage less than about 45% of the total volume of the biphasic biocompatible matrix. In some embodiments, the calcium phosphate has a volume percentage less than about 50% of the total volume of the biphasic biocompatible matrix.

In some embodiments of the present invention, the osseous phase comprises a calcium phosphate and an allograft material. In some embodiments, the osseous phase comprises a calcium sulfate and an allograft material. In some embodiments, the allograft material is a demineralized bone matrix.

In some embodiments, the osseous phase comprises a calcium phosphate, an allograft material, and collagen. In some embodiments, the osseous phase comprises β-tricalcium phosphate, an allograft material, and collagen. In some embodiments, the osseous phase comprises a calcium phosphate, a demineralized bone matrix, and collagen. In some embodiments, the osseous phase comprises β-tricalcium phosphate, a demineralized bone matrix, and collagen. In some embodiments, the osseous phase comprises calcium sulfate, an allograft material, and collagen. In some embodiments, the osseous phase comprises calcium sulfate, a demineralized bone matrix, and collagen.

In some embodiments, the osseous phase comprises an allograft material and collagen. In some embodiments, the osseous phase comprises a demineralized bone matrix and collagen.

In some embodiments of the present invention, the osseous phase forms a porous structure and comprises pores with a porosity greater than about 40%. In some embodiments, the osseous phase has a porosity greater than about 50%. In some embodiments, the osseous phase has a porosity greater than about 75%. In some embodiments, the osseous phase has a porosity greater than about 85%. In some embodiments, the osseous phase has a porosity greater than about 90%. In some embodiments, the osseous phase has a porosity greater than about 95%. In some embodiments, the osseous phase comprises a porous structure having pores that are interconnected. In some embodiments, the calcium phosphate in the osseous phase has interconnected pores. In some embodiments, the porosity is macroporosity.

In some embodiments of the present invention, the osseous phase forms a porous structure and comprises pores with a pore area size ranging from about from about 4500 μm² to about 20000 μm², and a pore perimeter size ranging from about 200 μm to about 500 μm. In some embodiments, the osseous phase forms a porous structure and comprises pores with a pore area size ranging from about 6000 μm² to about 15000 μm².

In some embodiments of the present invention, the porous structure of the osseous phase allows for infiltration of cells into pores of the osseous phase. In some embodiments, the osseous phase allows for attachment of cells. In some embodiments, the infiltrating or attached cells are mesenchymal stem cells (or marrow stromal cells). In some embodiments, the infiltrating or attached cells are osteoblasts. In some embodiments, the infiltrating or attached cells are chondrocytes.

In some embodiments of the present invention, the osseous phase is capable of increasing cell number or cell growth by about 100% to about 1000% (measured at about 2 days after cell seeding) in cells treated with PDGF in comparison to cells not treated with PDGF. In some embodiments, the osseous phase is capable of increasing cell number or cell growth by about 100% (measured at about 2 days after cell seeding) in cells treated with PDGF in comparison to cells not treated with PDGF. In some embodiments, the osseous phase is capable of increasing cell number or cell growth by about 200% (measured at about 2 days after cell seeding) in cells treated with PDGF in comparison to cells not treated with PDGF. In some embodiments, the osseous phase is capable of increasing cell number or cell growth by about 300% (measured at about 2 days after cell seeding) in cells treated with PDGF in comparison to cells not treated with PDGF. In some embodiments, the osseous phase is capable of increasing cell number or cell growth by about 400% (measured at about 2 days after cell seeding) in cells treated with PDGF in comparison to cells not treated with PDGF. In some embodiments, the osseous phase is capable of increasing cell number or cell growth by about 600% (measured at about 2 days after cell seeding) in cells treated with PDGF in comparison to cells not treated with PDGF. In some embodiments, the osseous phase is capable of increasing cell number or cell growth by about 800% (measured at about 2 days after cell seeding) in cells treated with PDGF in comparison to cells not treated with PDGF. In some embodiments, the osseous phase is capable of increasing cell number or cell growth by about 1000% (measured at about 2 days after cell seeding) in cells treated with PDGF in comparison to cells not treated with PDGF.

In some embodiments of the present invention, the trabecular number is increased by about 100% to about 1000% (measured at 12 weeks after administration of the matrix) in an individual treated with a composition comprising a biphasic biocompatible matrix and PDGF in comparison to an individual treated with a composition comprising the biphasic biocompatible matrix alone. In some embodiments, the trabecular number is increased by about 100% (measured at 12 weeks after administration of the matrix) in an individual treated with a composition comprising a biphasic biocompatible matrix and PDGF in comparison to an individual treated with a composition comprising the biphasic biocompatible matrix alone. In some embodiments, the trabecular number is increased by about 200% (measured at 12 weeks after administration of the matrix) in an individual treated with a composition comprising a biphasic biocompatible matrix and PDGF in comparison to an individual treated with a composition comprising the biphasic biocompatible matrix alone. In some embodiments, the trabecular number is increased by about 250% to about 1000% (measured at 12 weeks after administration of the matrix) in an individual treated with a composition comprising a biphasic biocompatible matrix and PDGF in comparison to an individual treated with a composition comprising the biphasic biocompatible matrix alone. In some embodiments, the trabecular number is increased by about 300% (measured at 12 weeks after administration of the matrix) in an individual treated with a composition comprising a biphasic biocompatible matrix and PDGF in comparison to an individual treated with a composition comprising the biphasic biocompatible matrix alone.

In some embodiments of the present invention, the cartilage phase comprises a glycosaminoglycan (GAG) and collagen. In some embodiments, the cartilage phase comprises a GAG and an allograft material. In some embodiments, the allograft material is not a demineralized bone matrix. In some embodiments, the allograft material is a mineralized bone matrix.

In some embodiments, the cartilage phase comprises a GAG, an allograft material, and collagen. In some embodiments, the cartilage phase comprises chondroitin sulfate, an allograft material, and collagen. In some embodiments, the cartilage phase comprises a GAG, a mineralized bone matrix, and collagen. In some embodiments, the cartilage phase comprises a chondroitin sulfate, a mineralized bone matrix, and collagen.

In some embodiments, the cartilage phase comprises collagen and a proteoglycan. In some embodiments, the cartilage phase comprises an allograft material and a proteoglycan. In some embodiments, the cartilage phase comprises a mineralized bone matrix and a proteoglycan.

In some embodiments, the cartilage phase comprises collagen, a proteoglycan, and an allograft material. In some embodiments, the cartilage phase comprises a mineralized bone matrix, a proteoglycan, and collagen.

In some embodiments of the present invention, the cartilage phase forms a porous structure and comprises pores with a porosity greater than about 40%. In some embodiments, the cartilage phase has a porosity greater than about 50%. In some embodiments, the cartilage phase has a porosity greater than about 75%. In some embodiments, the cartilage phase has a porosity greater than about 85%. In some embodiments, the cartilage phase has a porosity greater than about 90%. In some embodiments, the cartilage phase has a porosity greater than about 95%. In some embodiments, the cartilage phase comprises a porous structure having pores that are interconnected. In some embodiments, the porosity is macroporosity.

In some embodiments of the present invention, the cartilage phase forms a porous structure and comprises pores with a pore area size ranging from about from about 4500 μm² to about 20000 μm² and a pore perimeter size ranging from about 200 μm to about 500 μm. In some embodiments, the cartilage phase forms a porous structure and comprises pores with a pore area size ranging from about 6000 μm² to about 15000 μm².

In some embodiments of the present invention, the porous structure of the cartilage phase allows for infiltration of cells into pores of the cartilage phase. In some embodiments, the cartilage phase allows for attachment of cells. In some embodiments, the infiltrating or attached cells are mesenchymal stem cells (or marrow stromal cells). In some embodiments, the infiltrating or attached cells are osteoblasts. In some embodiments, the infiltrating or attached cells are chondrocytes.

In some embodiments of the present invention, the cartilage phase is capable of increasing cell number or cell growth by about 100% to about 1000% (measured at about 2 days after cell seeding) in cells treated with PDGF in comparison to cells not treated with PDGF. In some embodiments, the cartilage phase is capable of increasing cell number or cell growth by about 100% (measured at about 2 days after cell seeding) in cells treated with PDGF in comparison to cells not treated with PDGF. In some embodiments, the cartilage phase is capable of increasing cell number or cell growth by about 200% (measured at about 2 days after cell seeding) in cells treated with PDGF in comparison to cells not treated with PDGF. In some embodiments, the cartilage phase is capable of increasing cell number or cell growth by about 300% (measured at about 2 days after cell seeding) in cells treated with PDGF in comparison to cells not treated with PDGF. In some embodiments, the cartilage phase is capable of increasing cell number or cell growth by about 400% (measured at about 2 days after cell seeding) in cells treated with PDGF in comparison to cells not treated with PDGF. In some embodiments, the cartilage phase is capable of increasing cell number or cell growth by about 600% (measured at about 2 days after cell seeding) in cells treated with PDGF in comparison to cells not treated with PDGF. In some embodiments, the cartilage phase is capable of increasing cell number or cell growth by about 800% (measured at about 2 days after cell seeding) in cells treated with PDGF in comparison to cells not treated with PDGF. In some embodiments, the cartilage phase is capable of increasing cell number or cell growth by about 1000% (measured at about 2 days after cell seeding) in cells treated with PDGF in comparison to cells not treated with PDGF.

In some embodiments of the present invention, both the osseous phase and the cartilage phase are capable of increasing cell number or cell growth in both phases by about 100% to about 1000% (measured at about 2 days after cell seeding) in cells treated with PDGF in comparison to cells not treated with PDGF. In some embodiments, both the osseous phase and the cartilage phase are capable of increasing cell number or cell growth in both phases by about 100% (measured at about 2 days after cell seeding) in cells treated with PDGF in comparison to cells not treated with PDGF. In some embodiments, both the osseous phase and the cartilage phase are capable of increasing cell number or cell growth in both phases by about 200% (measured at about 2 days after cell seeding) in cells treated with PDGF in comparison to cells not treated with PDGF. In some embodiments, both the osseous phase and the cartilage phase are capable of increasing cell number or cell growth in both phases by about 300% (measured at about 2 days after cell seeding) in cells treated with PDGF in comparison to cells not treated with PDGF. In some embodiments, both the osseous phase and the cartilage phase are capable of increasing cell number or cell growth in both phases by about 400% (measured at about 2 days after cell seeding) in cells treated with PDGF in comparison to cells not treated with PDGF. In some embodiments, both the osseous phase and the cartilage phase are capable of increasing cell number or cell growth in both phases by about 600% (measured at about 2 days after cell seeding) in cells treated with PDGF in comparison to cells not treated with PDGF. In some embodiments, both the osseous phase and the cartilage phase are capable of increasing cell number or cell growth in both phases by about 800% (measured at about 2 days after cell seeding) in cells treated with PDGF in comparison to cells not treated with PDGF. In some embodiments, both the osseous phase and the cartilage phase are capable of increasing cell number or cell growth by about 1000% (measured at about 2 days after cell seeding) in cells treated with PDGF in comparison to cells not treated with PDGF.

In some embodiments of the present invention, the biphasic biocompatible matrix further comprises a biocompatible binder in the osseous and/or the cartilage phase.

In some embodiments of the present invention, the biphasic biocompatible matrix is bioresorbable. In some embodiments, the biphasic biocompatible matrix can be resorbed within about one year of in vivo administration. In some embodiments, the biphasic biocompatible matrix can be resorbed within about 1, 2, 3 4, 5, 6, 7, 8, 9, 10, or 11 months of in vivo administration. In some embodiments, the biphasic biocompatible matrix can be resorbed within about 30 days of in vivo administration. In some embodiments, the biphasic biocompatible matrix can be resorbed within about 10-14 days of in vivo administration. In some embodiments, the biphasic biocompatible matrix can be resorbed within about 10 days of in vivo administration. In some embodiments, the biphasic biocompatible matrix is resorbed such that at least about 70% to about 95% of the matrix is resorbed. In some embodiments, the biphasic biocompatible matrix is resorbed such that at least about 80% of the matrix is resorbed.

In some embodiments of the present invention, the biphasic biocompatible matrix allows for release of PDGF from the matrix. In some embodiments, the biphasic biocompatible matrix allows for release of at least about 70% of PDGF at 24 hrs. In some embodiments, the biphasic biocompatible matrix allows for release of at least about 71% of PDGF at 24 hrs. In some embodiments, the biphasic biocompatible matrix allows for release of at least about 72% of PDGF at 24 hrs. In some embodiments, the biphasic biocompatible matrix allows for release of at least about 73% of PDGF at 24 hrs. In some embodiments, the biphasic biocompatible matrix allows for release of at least about 74% of PDGF at 24 hrs. In some embodiments, the biphasic biocompatible matrix allows for release of at least about 75% of PDGF at 24 hrs.

In some embodiments of the present invention, the maximum gross score by area is increased by about 100% to about 500% (measured at 12 weeks after administration of the matrix) in an individual treated with a composition comprising a biphasic biocompatible matrix and PDGF in comparison to an individual treated with a composition comprising the biphasic biocompatible matrix alone. In some embodiments, the maximum gross score by area is increased by about 100% (measured at 12 weeks after administration of the matrix) in an individual treated with a composition comprising a biphasic biocompatible matrix and PDGF in comparison to an individual treated with a composition comprising the biphasic biocompatible matrix alone. In some embodiments, the maximum gross score by area is increased by about 200% (measured at 12 weeks after administration of the matrix) in an individual treated with a composition comprising a biphasic biocompatible matrix and PDGF in comparison to an individual treated with a composition comprising the biphasic biocompatible matrix alone. In some embodiments, the maximum gross score by area is increased by about 300% (measured at 12 weeks after administration of the matrix) in an individual treated with a composition comprising a biphasic biocompatible matrix and PDGF in comparison to an individual treated with a composition comprising the biphasic biocompatible matrix alone.

In some embodiments, the biphasic biocompatible matrix is capable of absorbing an amount of a solution comprising PDGF that is between a range of about 25% to about 2000% by weight of the biphasic biocompatible matrix. In some embodiments, the biphasic biocompatible matrix is capable of absorbing an amount of a solution comprising PDGF that is between a range of about 100% to about 1600% by weight of the biphasic biocompatible matrix. In some embodiments, the biphasic biocompatible matrix is capable of absorbing an amount of a solution comprising PDGF that is equal to at least about 25% by weight of the biphasic biocompatible matrix. In some embodiments, the biphasic biocompatible matrix is capable of absorbing an amount of a solution comprising PDGF that is equal to at least about 100% by weight of the biphasic biocompatible matrix. In some embodiments, the biphasic biocompatible matrix is capable of absorbing an amount of a solution comprising PDGF that is equal to at least about 500% by weight of the biphasic biocompatible matrix. In some embodiments, the biphasic biocompatible matrix is capable of absorbing an amount of a solution comprising PDGF that is equal to at least about 1000% by weight of the biphasic biocompatible matrix. In some embodiments, the biphasic biocompatible matrix is capable of absorbing an amount of a solution comprising PDGF that is equal to at least about 1550% by weight of the biphasic biocompatible matrix. In some embodiments, the biphasic biocompatible matrix is capable of absorbing an amount of a solution comprising PDGF that is equal to at least about 1600% by weight of the biphasic biocompatible matrix. In some embodiments, the biphasic biocompatible matrix is capable of absorbing an amount of a solution comprising PDGF that is equal to at least about 2000% by weight of the biphasic biocompatible matrix.

In some embodiments of the present invention, are provided compositions and methods for treating osteoarthritis.

In some embodiments of the present invention, PDGF is present in a solution and is at a concentration in the range of about 0.01 mg/ml to about 10.0 mg/ml. In some embodiments, PDGF is present in a solution and is at a concentration in the range of about 0.01 mg/ml to about 1.0 mg/ml. In some embodiments, PDGF is present in a solution and is at a concentration in the range of about 0.01 mg/ml to about 2.0 mg/ml. In some embodiments, PDGF is present in a solution and is at a concentration in the range of about 0.01 mg/ml to about 3.0 mg/ml. In some embodiments, PDGF is present in a solution and is at a concentration in the range of about 0.05 mg/ml to about 5.0 mg/ml. In some embodiments, PDGF is present in a solution and is at a concentration in the range of about 0.1 mg/ml to about 5.0 mg/ml. In some embodiments, PDGF is present in a solution and is at a concentration in the range of about 0.1 mg/ml to about 3.0 mg/ml. In some embodiments, PDGF is at a concentration in the range of about 0.1 mg/ml to about 1.0 mg/ml. In some embodiments, PDGF is at a concentration of about 0.03 mg/ml, about 0.15 mg/ml, about 0.3 mg/ml, or about 1.0 mg/ml.

In some embodiments of the present invention, PDGF is present in a solution and is at an amount in the range of about 1 μg to about 50 mg, about 1 μg to about 10 mg, about 1 μg to about 1 mg, about 1 μg to about 500 μg, about 10 μg to about 25 mg, about 10 μg to about 500 μg, about 100 μg to about 10 mg, or about 250 μg to about 5 mg. In some embodiments, PDGF is at an amount of about 15 μg, about 75 μg, about 150 μg, or about 500 μg.

In some embodiments of the present invention, the method may be performed using open or mini-open arthroscopic techniques, endoscopic techniques, laparoscopic techniques, or any other suitable minimally-invasive techniques.

In some embodiments of the present invention, PDGF is a PDGF homodimer. In some embodiments, PDGF is a heterodimer. Examples of PDGF include PDGF-AA, PDGF-BB, PDGF-AB, PDGF-CC, PDGF-DD, and mixtures and derivatives thereof. In some embodiments, PDGF comprises PDGF-BB. In some embodiments, PDGF comprises a recombinant human (rh) PDGF such as recombinant human PDGF-BB (rhPDGF-BB).

In some embodiments of the present invention, PDGF is a PDGF fragment. In some embodiments, rhPDGF-B comprises the following fragments: amino acid sequences 1-31, 1-32, 33-108, 33-109, and/or 1-108 of the entire B chain.

In some embodiments, it is to be understood that one, some, or all of the properties of the various embodiments described herein may be combined to form some embodiments of the present invention.

### BRIEF DESCRIPTION OF THE FIGURES

FIGS. 1A-1O depict the physical characteristics of a biphasic matrix plug (Chondromimetic, Orthomimetic®, Cambridge, United Kingdom) by scanning electron microscopy: FIGS. 1A-1F (top surface of plug material); FIGS. 1G-1I (top phase of plug material); FIGS. 1J-1K (top phase on left/bottom phase on right—vertical cut through plug material, interior surface); and FIGS. 1L-1O (bottom phase).

FIG. 2 depicts changes in size in plug material over 96 hours.

FIG. 3 depicts the steps of loading of rhPDGF-BB on a biphasic matrix disc.

FIGS. 4A-4B depict cumulative release (ng or % release) profile of rhPDGF-BB from the Chondromimetic biphasic matrix plug combined with rhPDGF-BB over 24 hours at 37° C. as compared to control rhPDGF-BB sample.

FIG. 5A shows recovery of rhPDGF in eluates from the biphasic matrix plug at different salt concentrations. Averages of two experiments are shown.

FIG. 5B depicts binding curves of rhPDGF-BB eluted from the biphasic matrix plugs at different salt concentrations. ELISA assay was performed at eight different concentrations of rhPDGF-BB in duplicates. Negative controls (no receptor coated to the plate) were subtracted.

FIG. 6 depicts the steps of cell (human marrow stromal cells (hMSC)) seeding onto a biphasic matrix disc.

FIGS. 7A-7F show the physical characteristics of a biphasic matrix disc with or without cell seeding by scanning electron microscopy. FIGS. 7A-7C depict the lower phase of the biphasic matrix comprising cross-linked fibers with a calcium phosphate coating without hMSC cells (FIGS. 7A-7B) or with hMSC cells (FIG. 7C). The top layer parallel fiber alignment is shown without hMSC cells (FIGS. 7D-7E) or with hMSC cells (FIG. 7F).

FIG. 8 shows the result of luminescent cell viability ATP assay. Error bars represent the standard deviation. Statistical significance (P<0.05) between the rhPDGF-BB treated and control groups for both the top and lower phases are shown.

FIGS. 9A-9E depict maximum gross score by area for each specimen within each treatment group: 9A: empty defect treatment group; 9B: 0 μg rhPDGF-BB treatment group; 9C: 15 μg rhPDGF-BB treatment group; 9D: 75 μg rhPDGF-BB treatment group; 9E: 500 μg rhPDGF-BB treatment group.

FIG. 10 shows gross articular cartilage repair evaluation of rhPDGF-BB treatment groups, Maximum score by area. *: Indicates significant difference (p<0.05) compared to the Empty Defect treatment group. ‡: Indicates significant difference compared to Empty Defect, 0 μg rhPDGF-BB, and 15 μg rhPDGF-BB treatment groups.

FIGS. 11A-11F show the trabecular number (1/mm), trabecular thickness (mm), or bone volume (mm³) of rhPDGF-BB treatment groups by microtomography (microCT). FIG. 11A depicts trabecular number (1/mm) of 8 mm×6.25 mm contour of rhPDGF-BB treatment groups by microCT. FIG. 11B depicts bone volume (mm³) of 8 mm×6.25 mm contour of rhPDGF-BB treatment groups by microCT. FIG. 11C depicts trabecular number (1/mm) of 8 mm×7.5 mm depth contour of rhPDGF-BB treatment groups by microCT. FIG. 11D depicts trabecular thickness (mm) of 4 mm×6.25 mm depth contour of rhPDGF-BB treatment groups by microCT. FIG. 11E depicts bone volume (mm³) of 4 mm diameter×6.25 mm depth contour of rhPDGF-BB treatment groups by microCT. FIG. 11F depicts bone volume (mm³) of 6 mm diameter×6.25 mm depth contour of rhPDGF-BB treatment groups by microCT. *: Indicates significant difference p<0.05.

### DETAILED DESCRIPTION OF THE INVENTION

Inventors have discovered that a composition comprising a biphasic biocompatible matrix having an osseous phase and a cartilage phase in combination with platelet derived growth factor (PDGF) augments or enhances subchondral bone and cartilage repair. In some embodiments, the composition is capable of significantly increasing trabecular number and/or enhancing bony bridging in a subject in comparison to a subject being treated without the composition. In some embodiments, the composition is capable of enhancing gross articular cartilage repair, for example, as evidenced by an increase in the maximum gross score by area in a subject treated with such composition. In some embodiments, the composition allows for increased release of PDGF. In some embodiments, both the osseous phase and the cartilage phase are capable of increasing cell number or cell growth in cells treated with PDGF in comparison to cells not treated with PDGF.

Without wishing to be bound by theory, a composition comprising a biphasic biocompatible matrix having an osseous phase and a cartilage phase in combination with platelet derived growth factor (PDGF) may increase the formation of cartilage and bone in osteochondral defects, e.g., through recruitment of stem cells, increased synthesis of appropriate collagen subtypes and bone ingrowth, and/or by providing a framework or scaffold for new bony tissue ingrowth and the cartilage regeneration.

The present invention provides for compositions and methods for treating an osteochondral defect, in a cartilage and/or in a bone adjacent to the cartilage. In one aspect of the invention, a composition is provided comprising a biphasic biocompatible matrix and platelet derived growth factor (PDGF), wherein the biphasic biocompatible matrix comprises a scaffolding material and wherein the scaffolding material forms a porous structure comprising an osseous phase and a cartilage phase.

In another aspect of the invention, provided are methods for treating an osteochondral defect in a cartilage and/or a bone adjacent to the cartilage in an individual comprising administering to said individual an effective amount of a composition comprising a biphasic biocompatible matrix and platelet derived growth factor (PDGF) to at least one site of the osteochondral defect, wherein the biphasic biocompatible matrix comprises a scaffolding material and wherein the scaffolding material forms a porous structure comprising an osseous phase and a cartilage phase.

For purposes of interpreting this specification, the following definitions will apply and whenever appropriate, terms used in the singular will also include the plural and vice versa. In the event that any definition set forth below conflicts with any document incorporated herein by reference, the definition set forth below shall control.

As used herein, the terms “bone” or “bone adjacent to the cartilage,” which may be treated by compositions and methods of the present invention, comprise a subchondral bone or a cancellous (also known as trabecular) bone.

An “individual” refers a mammal, including humans, domestic and farm animals, and zoo, sport, or pet animals, such as chimpanzees and other apes and monkey species, dogs, horses, rabbits, cattle, pigs, goats, sheep, hamsters, guinea pigs, gerbils, mice, ferrets, rats, cats, and the like. In some embodiments, the individual is human. The term does not denote a particular age or gender.

An “effective amount” refers to at least an amount effective, at a dosage and for a period of time necessary, to achieve a desired therapeutic or clinical result. An effective amount can be provided in one or more administrations.

“Bioresorbable” refers to the ability of a biocompatible matrix to be resorbed or remodeled in vivo. The resorption process involves degradation and elimination of the original material through the action of body fluids, enzymes or cells. The resorbed material may be used by the host in the formation of new tissue, or it may be otherwise re-utilized by the host, or it may be excreted.

Collagen, as referred to herein, are materials in the form of gels, particles, powders, sheets, patches, pads, plugs, or sponges. Collagen may be manufactured from collagen extracts of, for example, bovine dermis or bovine Achilles tendon. Collage may also be made from collagen slurries where the concentration of the collagen in the slurry is different for each type of osseous phase and cartilage phase. For example, collagen can be made from a slurry with a collagen concentration of about 4.5%, about 5%, about 6%, or about 7%. For any biphasic biocompatible matrix, the percent of collagen used in the starting slurry does not reflect the percentage of collagen in the final osseous phase or cartilage phase in the biphasic biocompatible matrix.

As used herein, unless otherwise specified, the term “treatment” or “treating” refers to administrating to an individual a composition comprising a biphasic biocompatible matrix and platelet-derived growth factor which obtain beneficial or desired clinical results for which the subject is being treated. For purpose of this invention, beneficial or desired clinical results include, but are not limited to, alleviation of one or more symptoms associated with osteochondral injuries or defects, diminishment of extent of osteochondral injuries or defects, stabilizing (i.e., not worsening) one or more symptoms associated with osteochondral injuries or defects, delaying or slowing of osteochondral injuries or defects progression, amelioration or palliation of the osteochondral injuries or defects state, increased rate of healing process of osteochondral injuries or defects, and partial or total remission, whether detectable or undetectable. An example of osteochondral injuries or defects is osteoarthritis. Treating an osteochondral defect may involve treating a cartilage, a bone adjacent to the cartilage, or both, and the beneficial or desired clinical results may include beneficial or desired clinical results in the cartilage, the bone adjacent to the cartilage, or both. “Treatment” can also mean prolonging survival as compared to expected survival if not receiving treatment. In some embodiments, “treatment” of osteochondral injuries or defects can encompass curing a disease. In some embodiments, beneficial or desired results with respect to a condition include, but are not limited to, improving a condition, curing a condition, lessening severity of a condition, delaying progression of a condition, alleviating one or more symptoms associated with a condition, increasing the quality of life of one suffering from a condition, and/or prolonging survival.

As used herein, the term “allograft material” refers to a transplanted tissue or cell that is sourced from a genetically non-identical member of the same species. An allograft material can be used in its native state or a modified state. For example, an allograft material may be a mineralized bone matrix, a demineralized bone matrix, or a partially demineralized bone matrix (e.g., sponges or sheets). Demineralized bone matrix as used herein refers to a mineralized bone material which has been treated for removal of minerals within the bone. As used herein, the singular forms “a”, “an”, and “the” include plural references unless indicated otherwise.

Reference to “about” a value or parameter herein includes (and describes) embodiments that are directed to that value or parameter per se. For example, description referring to “about X” includes description of “X,” as well as “about X.”

It is understood that aspects and embodiments of the invention described herein include “comprising,” “consisting,” and “consisting essentially of” aspects and embodiments.

### Compositions and Methods of the Invention

Described here are compositions and methods for treating an osteochondral defect in a cartilage and a bone. In one aspect of the invention, a composition is provided comprising a biphasic biocompatible matrix and platelet derived growth factor (PDGF), wherein the biphasic biocompatible matrix comprises a scaffolding material and wherein the scaffolding material forms a porous structure comprising an osseous phase and a cartilage phase.

In some embodiments, provided is a composition for treating an osteochondral defect in a cartilage and/or a bone adjacent to the cartilage comprising a biphasic biocompatible matrix and PDGF, wherein the biphasic biocompatible matrix comprises a scaffolding material, wherein the scaffolding material forms a porous structure comprising an osseous and a cartilage phase, wherein the PDGF is in a solution, wherein the PDGF solution has a concentration of PDGF ranging from about 0.01 mg/ml to about 10 mg/ml. In some embodiments, the PDGF solution has a concentration of about 1.0 mg/ml. In some embodiments, the weight/weight ratio between the osseous phase and the cartilage phase in a scaffolding matrix of a biphasic biocompatible matrix is between about 65:35 to about 99:1.

In some embodiments, provided is a composition for treating an osteochondral defect in a cartilage and/or a bone adjacent to the cartilage consisting of a biphasic biocompatible matrix and PDGF, wherein the biphasic biocompatible matrix consisting of a scaffolding material, wherein the scaffolding material forms a porous structure consisting of an osseous and a cartilage phase, wherein the PDGF is in a solution, wherein the PDGF solution has a concentration of PDGF ranging from about 0.01 mg/ml to about 10 mg/ml. In some embodiments, the PDGF solution has a concentration of about 1.0 mg/ml. In some embodiments, the weight/weight ratio between the osseous phase and the cartilage phase in a scaffolding matrix of a biphasic biocompatible matrix is between about 65:35 to about 99:1.

In another aspect of the invention, provided are methods for treating an osteochondral defect in a cartilage and a bone adjacent to the cartilage in an individual comprising administering to the individual an effective amount of a composition comprising a biphasic biocompatible matrix and platelet derived growth factor (PDGF) to at least one site of the osteochondral defect, wherein the biphasic biocompatible matrix comprises a scaffolding material and wherein the scaffolding material forms a porous structure comprising an osseous phase and a cartilage phase.

In some embodiments, provided is a method for treating an osteochondral defect in a cartilage and a bone adjacent to the cartilage in an individual comprising administering to the individual an effective amount of a composition comprising a biphasic biocompatible matrix and platelet derived growth factor (PDGF) to at least one site of the osteochondral defect, wherein the biphasic biocompatible matrix comprises a scaffolding material and wherein the scaffolding material forms a porous structure comprising an osseous phase and a cartilage phase, wherein the PDGF is in a solution, wherein the PDGF solution has a concentration of PDGF ranging from about 0.01 mg/ml to about 10 mg/ml. In some embodiments, the PDGF solution has a concentration of about 1.0 mg/ml. In some embodiments, the weight/weight ratio between the osseous phase and the cartilage phase in a scaffolding matrix of a biphasic biocompatible matrix is between about 65:35 to about 99:1.

In some embodiments, provided is a method for treating an osteochondral defect in a cartilage and a bone adjacent to the cartilage in an individual consisting of administering to the individual an effective amount of a composition consisting of a biphasic biocompatible matrix and platelet derived growth factor (PDGF) to at least one site of the osteochondral defect, wherein the biphasic biocompatible matrix consisting of a scaffolding material and wherein the scaffolding material forms a porous structure consisting of an osseous phase and a cartilage phase, wherein the PDGF is in a solution, wherein the PDGF solution has a concentration of PDGF ranging from about 0.01 mg/ml to about 10 mg/ml. In some embodiments, the PDGF solution has a concentration of about 1.0 mg/ml. In some embodiments, the weight/weight ratio between the osseous phase and the cartilage phase in a scaffolding matrix of a biphasic biocompatible matrix is between about 65:35 to about 99:1.

### Biphasic Biocompatible Matrix

A biphasic biocompatible matrix, according to embodiments of the present invention, comprises a dual-layer or a biphasic scaffolding material. In some embodiments, the scaffolding material forms a porous structure comprising an osseous phase and a cartilage phase. The osseous phase and the cartilage phase provide a framework or scaffold for new bony tissue ingrowth and the cartilage regeneration, respectively. Cartilage regeneration includes cartilaginous tissue growth in an articular cartilage, a fibrocartilage, or an elastic cartilage. Bone ingrowth includes bone growth in a subchondral or a cancellous (also known as trabecular) bone.

Cartilage, according to embodiments of the present invention, comprises an articular cartilage, a fibrocartilage, or an elastic cartilage. Articular cartilage (or hyaline cartilage) is the smooth, glistening white tissue that covers the surface of all the diarthrodial joints including, but not limited to, knee joint (e.g., femur, tibia, femoral condyle), glenohumeral and elbow joints, radioulnar joint, interphalangeal joint, talus (e.g., foot and ankle), and hip.

In some embodiments, the osseous phase comprises at least one calcium phosphate. In some embodiments, the osseous phase comprises a plurality of calcium phosphates. In some embodiments, the calcium phosphate used in the osseous phase has a calcium to phosphorus atomic ratio ranging from about 0.5 to about 2.0. In some embodiments, the calcium phosphate used in the osseous phase consists of particles in a range of about 100 μm to about 5000 μm in size. In some embodiments, the calcium phosphate consists of particles in a range of about 100 μm to about 3000 μm in size. In some embodiments, the calcium phosphate consists of particles in a range of about 250 μm to about 1000 μm in size.

In some embodiments, the calcium phosphate used in the osseous phase has a lower volume percentage in comparison to the total of volume of the biphasic biocompatible matrix. In some embodiments, the calcium phosphate has a volume percentage ranging from about less than about 5% to about less than 50% of the total volume of the biphasic biocompatible matrix. In some embodiments, the calcium phosphate has a volume percentage less than about 5% of the total volume of the biphasic biocompatible matrix. In some embodiments, the calcium phosphate has a volume percentage less than about 10% of the total volume of the biphasic biocompatible matrix. In some embodiments, the calcium phosphate has a volume percentage less than about 15% of the total volume of the biphasic biocompatible matrix. In some embodiments, the calcium phosphate has a volume percentage less than about 20% of the total volume of the biphasic biocompatible matrix. In some embodiments, the calcium phosphate has a volume percentage less than about 30% of the total volume of the biphasic biocompatible matrix. In some embodiments, the calcium phosphate has a volume percentage less than about 35% of the total volume of the biphasic biocompatible matrix. In some embodiments, the calcium phosphate has a volume percentage less than about 40% of the total volume of the biphasic biocompatible matrix. In some embodiments, the calcium phosphate has a volume percentage less than about 45% of the total volume of the biphasic biocompatible matrix. In some embodiments, the calcium phosphate has a volume percentage less than about 50% of the total volume of the biphasic biocompatible matrix.

Calcium phosphates suitable for use in an osseous phase include, but are not limited to amorphous calcium phosphate, monocalcium phosphate monohydrate (MCPM), monocalcium phosphate anhydrous (MCPA), dicalcium phosphate dihydrate (DCPD), dicalcium phosphate anhydrous (DCPA), octacalcium phosphate (OCP), α-tricalcium phosphate (α-TCP), β-tricalcium phosphate (β-TCP), hydroxyapatite (OHAp), poorly crystalline hydroxyapatite, tetracalcium phosphate (TTCP), heptacalcium decaphosphate, calcium metaphosphate, calcium pyrophosphate dehydrate, carbonated calcium phosphate, and calcium pyrophosphate. In some embodiments, the calcium phosphate is β-TCP.

In some embodiments, the osseous phase comprises at least one calcium sulfate. In some embodiments, the osseous phase comprises a plurality of calcium sulfates.

Calcium sulfates suitable for use in an osseous phase include, but are not limited to, γ-anhydrite, hemihydrate (α-hemihydrate, and β-hemihydrate), gypsum (dehydrate), β-anhydrite, and calcium sulfate dehydrate.

In some embodiments, the osseous phase comprises collagen. In some embodiments, the collagen comprises Type I, II, III, or IV collagen. In some embodiments, the collagen comprises a mixture of collagens, such as a mixture of Type I and Type II collagen. In some embodiments, the collagen comprises Type II collagen. In some embodiments, the collagen comprises, for example, a fibrous collagen such as soluble Type II bovine dermis-derived or tendon-derived collagen. Collagen may comprise a fibrous collagen such as soluble Type II fibrous collagen in collagen gels, particles, powders, patches, pads, sheets, plugs, or sponges and, in some embodiments, may demonstrate sufficient mechanical properties, including wet tensile strength, to withstand suturing and hold a suture without tearing. In some embodiments, the collagen has a density ranging from about 0.75 g/cm³ to about 1.5 g/cm³.

In some embodiments, the collagen is soluble under physiological conditions. In some embodiments, the collagen is soluble and cross-linked under physiological conditions. In some embodiments, the collagen comprises fibrous and acid-soluble collagen derived from bovine dermal tissue or bovine Achilles tissue. A fibrous collagen, for example, can have a wet tear strength ranging from about 0.75 pounds to about 5 pounds. Other types of collagen present in bone or musculoskeletal tissues may be employed. Recombinant, synthetic, and naturally occurring forms of collagen may be used in the present invention.

In some embodiments, the collagen is obtained from a commercial source and is made from purified collagen extract from bovine dermis or bovine tendon. In some embodiments, the collagen is Type II bovine collagen. In some embodiments, the collagen is made from a collagen slurry with any one of the following concentrations of collagen (w/v): about 4.5%, about 5%, about 6% or about 7%.

In some embodiments, the osseous phase comprises an allograft material. Without wishing to be bound by theory, an allograft material may function to prevent delamination of the forming clot and immature tissue, recruitment of cells, and drive the synthesis of cartilage (e.g., articular cartilage, fibrocartilage, or elastic cartilage) and its underlying bone. An allograft material can be a mineralized bone matrix, a demineralized bone matrix, or a partially demineralized bone matrix. In some embodiments, the allograft material for the osseous phase is a demineralized bone matrix. In some embodiments, the allograft material for the osseous phase is a partially demineralized bone matrix.

In some embodiments, the osseous phase comprises a calcium phosphate and collagen. In some embodiments, the osseous phase comprises β-tricalcium phosphate and collagen. In some embodiments, the osseous phase comprises a calcium sulfate and collagen.

In some embodiments, the osseous phase comprises a calcium phosphate and an allograft material. In some embodiments, the osseous phase comprises β-tricalcium phosphate and an allograft material. In some embodiments, the osseous phase comprises a calcium phosphate and a demineralized bone matrix. In some embodiments, the osseous phase comprises β-tricalcium phosphate and a demineralized bone matrix. In some embodiments, the osseous phase comprises calcium sulfate and an allograft material. In some embodiments, the osseous phase comprises calcium sulfate and a demineralized bone matrix.

In some embodiments, the osseous phase comprises a calcium phosphate, an allograft material, and collagen. In some embodiments, the osseous phase comprises β-tricalcium phosphate, an allograft material, and collagen. In some embodiments, the osseous phase comprises a calcium phosphate, a demineralized bone matrix, and collagen. In some embodiments, the osseous phase comprises β-tricalcium phosphate, a demineralized bone matrix, and collagen. In some embodiments, the osseous phase comprises calcium sulfate, an allograft material, and collagen. In some embodiments, the osseous phase comprises calcium sulfate, a demineralized bone matrix, and collagen.

In some embodiments, the osseous phase comprises an allograft material, and collagen. In some embodiments, the osseous phase comprises a demineralized bone matrix, and collagen.

In some embodiments, the osseous phase forms a porous structure. In some embodiments, the osseous phase forms a porous structure and comprises pores with a pore area size ranging from about 4500 μm² to about 20000 μm², and a pore perimeter size ranging from about 200 μm to about 500 μm. In some embodiments, the osseous phase forms a porous structure and comprises pores with a pore area size ranging from about 6000 μm² to about 15000 μm² (see U.S. 61/191,641, hereby incorporated by reference by its entirety).

In some embodiments, the osseous phase forms a porous structure and comprises pores with a porosity greater than about 40%. In some embodiments, the osseous phase has a porosity greater than about 50%. In some embodiments, the osseous phase has a porosity greater than about 75%. In some embodiments, the osseous phase has a porosity greater than about 80%. In some embodiments, the osseous phase has a porosity greater than about 85%. In some embodiments, the osseous phase has a porosity greater than about 90%. In some embodiments, the osseous phase has a porosity greater than about 95%.

In some embodiments of the present invention, the porous structure of the osseous phase allows for infiltration of cells into pores of the osseous phase. In some embodiments, the osseous phase allows for attachment of cells. In some embodiments, the infiltrating or attached cells are mesenchymal stem cells (or marrow stromal cells). In some embodiments, the infiltrating or attached cells are osteoblasts. In some embodiments, the infiltrating or attached cells are chondrocytes.

In some embodiments of the present invention, the osseous phase is capable of increasing cell number or cell growth by about 100% to about 1000% (measured at about 2 days after cell seeding) in cells treated with PDGF in comparison to cells not treated with PDGF. In some embodiments, the osseous phase is capable of increasing cell number or cell growth by about 100% (measured at about 2 days after cell seeding) in cells treated with PDGF in comparison to cells not treated with PDGF. In some embodiments, the osseous phase is capable of increasing cell number or cell growth by about 200% (measured at about 2 days after cell seeding) in cells treated with PDGF in comparison to cells not treated with PDGF. In some embodiments, the osseous phase is capable of increasing cell number or cell growth by about 300% (measured at about 2 days after cell seeding) in cells treated with PDGF in comparison to cells not treated with PDGF. In some embodiments, the osseous phase is capable of increasing cell number or cell growth by about 400% (measured at about 2 days after cell seeding) in cells treated with PDGF in comparison to cells not treated with PDGF. In some embodiments, the osseous phase is capable of increasing cell number or cell growth by about 600% (measured at about 2 days after cell seeding) in cells treated with PDGF in comparison to cells not treated with PDGF. In some embodiments, the osseous phase is capable of increasing cell number or cell growth by about 800% (measured at about 2 days after cell seeding) in cells treated with PDGF in comparison to cells not treated with PDGF. In some embodiments, the osseous phase is capable of increasing cell number or cell growth by about 1000% (measured at about 2 days after cell seeding) in cells treated with PDGF in comparison to cells not treated with PDGF.

In some embodiments of the present invention, the trabecular number is increased by about 100% to about 1000% (measured at 12 weeks after administration of the matrix) in an individual treated with a composition comprising a biphasic biocompatible matrix and PDGF in comparison to an individual treated with a composition comprising the biphasic biocompatible matrix alone. In some embodiments, the trabecular number is increased by about 100% (measured at 12 weeks after administration of the matrix) in an individual treated with a composition comprising a biphasic biocompatible matrix and PDGF in comparison to an individual treated with a composition comprising the biphasic biocompatible matrix alone. In some embodiments, the trabecular number is increased by about 200% (measured at 12 weeks after administration of the matrix) in an individual treated with a composition comprising a biphasic biocompatible matrix and PDGF in comparison to an individual treated with a composition comprising the biphasic biocompatible matrix alone. In some embodiments, the trabecular number is increased by about 250% to about 1000% (measured at 12 weeks after administration of the matrix) in an individual treated with a composition comprising a biphasic biocompatible matrix and PDGF in comparison to an individual treated with a composition comprising the biphasic biocompatible matrix alone. In some embodiments, the trabecular number is increased by about 300% (measured at 12 weeks after administration of the matrix) in an individual treated with a composition comprising a biphasic biocompatible matrix and PDGF in comparison to an individual treated with a composition comprising the biphasic biocompatible matrix alone. In some embodiments, the trabecular number is increased by about 400% (measured at 12 weeks after administration of the matrix) in an individual treated with a composition comprising a biphasic biocompatible matrix and PDGF in comparison to an individual treated with a composition comprising the biphasic biocompatible matrix alone. In some embodiments, the trabecular number is increased by about 500% (measured at 12 weeks after administration of the matrix) in an individual treated with a composition comprising a biphasic biocompatible matrix and PDGF in comparison to an individual treated with a composition comprising the biphasic biocompatible matrix alone. In some embodiments, the trabecular number is increased by about 600% (measured at 12 weeks after administration of the matrix) in an individual treated with a composition comprising a biphasic biocompatible matrix and PDGF in comparison to an individual treated with a composition comprising the biphasic biocompatible matrix alone. In some embodiments, the trabecular number is increased by about 750% (measured at 12 weeks after administration of the matrix) in an individual treated with a composition comprising a biphasic biocompatible matrix and PDGF in comparison to an individual treated with a composition comprising the biphasic biocompatible matrix alone. In some embodiments, the trabecular number is increased by about 1000% (measured at 12 weeks after administration of the matrix) in an individual treated with a composition comprising a biphasic biocompatible matrix and PDGF in comparison to an individual treated with a composition comprising the biphasic biocompatible matrix alone.

In some embodiments, the cartilage phase comprises collagen. In some embodiments, the collagen comprises Type I, II, III, or IV collagen. In some embodiments, the collagen comprises a mixture of collagens, such as a mixture of Type I and Type II collagen. In some embodiments, the collagen comprises Type II collagen. In some embodiments, the collagen comprises a fibrous collagen such as soluble type II bovine dermis-derived or tendon-derived collagen. Collagen may comprise, for example, a fibrous collagen such as soluble type II fibrous collagen suitable for use in collagen gels, particles, powders, patches, pads, sheets, plugs, or sponges and in some embodiments, may demonstrate sufficient mechanical properties, including wet tensile strength, to withstand suturing and hold a suture without tearing. In some embodiments, the collagen has a density ranging from about 0.75 g/cm³ to about 1.5 g/cm³.

In some embodiments, the cartilage phase comprises a glycosaminoglycan (GAG or mucopolysaccharides). In some embodiments, the GAG is chondroitin sulfate. Other GAGs suitable for use in the invention include, but are not limited to, dermatan sulfate, keratan sulfate, heparin, heparin sulfate, hyaluronan, and combinations thereof. In some embodiments, the weight/weight ratio of collagen to GAG in the cartilage phase is between about 70:30 to about 95:5. In some embodiments, the weight/weight ratio of collagen to GAG in a cartilage phase is about 90:10. In some embodiments, the weight/weight ratio of collagen to GAG in a cartilage phase is about 95:5.

In some embodiments, the cartilage phase comprises a proteoglycan. In some embodiments, the proteoglycan is an aggrecan. In some embodiments, the weight/weight ratio of collagen to proteoglycan in the cartilage phase is between about 70:30 to about 95:5. In some embodiments, the weight/weight ratio of collagen to proteoglycan in a cartilage phase is about 90:10. In some embodiments, the weight/weight ratio of collagen to proteoglycan in a cartilage phase is about 95:5.

In some embodiments, the cartilage phase comprises an allograft material. Without wishing bound by theory, an allograft may function to prevent delamination of the forming clot and immature tissue, recruitment of cells, and drive the synthesis of cartilage (e.g., an articular cartilage, a fibrocartilage, or an elastic cartilage) and its underlying bone. An allograft material comprises, for example, a mineralized bone matrix, a demineralized bone matrix, or a partial demineralized bone matrix. In some embodiments, the allograft material for a cartilage phase is a mineralized bone matrix.

In some embodiments, the cartilage phase comprises a GAG and collagen. In some embodiments, the cartilage phase comprises chondroitin sulfate and collagen.

In some embodiments, the cartilage phase comprises a GAG and an allograft material. In some embodiments, the cartilage phase comprises chondroitin sulfate and an allograft material. In some embodiments, the cartilage phase comprises a GAG and a mineralized bone matrix. In some embodiments, the cartilage phase comprises a chondroitin sulfate and a mineralized bone matrix.

In some embodiments, the cartilage phase comprises a GAG, an allograft material, and collagen. In some embodiments, the cartilage phase comprises chondroitin sulfate, an allograft material, and collagen. In some embodiments, the cartilage phase comprises a GAG, a mineralized bone matrix, and collagen. In some embodiments, the cartilage phase comprises a chondroitin sulfate, a mineralized bone matrix, and collagen.

In some embodiments, the cartilage phase comprises collagen and a proteoglycan. In some embodiments, the cartilage phase comprises an allograft material and a proteoglycan. In some embodiments, the cartilage phase comprises a mineralized bone matrix and a proteoglycan.

In some embodiments, the cartilage phase comprises collagen, a proteoglycan, and an allograft material. In some embodiments, the cartilage phase comprises a mineralized bone matrix, a proteoglycan, and collagen.

In some embodiments, the cartilage phase forms a porous structure. In some embodiments, the cartilage phase forms a porous structure and comprises pores with a pore area size ranging from about 4500 μm² to about 20000 μm² and a pore perimeter size ranging from about 200 μm to about 500 μm. In some embodiments, the cartilage phase forms a porous structure and comprises pores with a pore area size ranging from about 6000 μm² to about 15000 μm² (see U.S. 61/191,641, hereby incorporated by reference by its entirety).

In some embodiments, the cartilage phase forms a porous structure and comprises pores with a porosity greater than about 40%. In some embodiments, the cartilage phase has a porosity greater than about 50%. In some embodiments, the cartilage phase has a porosity greater than about 75%. In some embodiments, the cartilage phase has a porosity greater than about 80%. In some embodiments, the cartilage phase has a porosity greater than about 85%. In some embodiments, the cartilage phase has a porosity greater than about 90%. In some embodiments, the cartilage phase has a porosity greater than about 95%.

In some embodiments of the present invention, the porous structure of the cartilage phase allows for infiltration of cells into pores of the cartilage phase. In some embodiments, the cartilage phase allows for attachment of cells. In some embodiments, the infiltrating or attached cells are mesenchymal stem cells (or marrow stromal cells). In some embodiments, the infiltrating or attached cells are osteoblasts. In some embodiments, the infiltrating or attached cells are chondrocytes.

In some embodiments of the present invention, the cartilage phase is capable of increasing cell number or cell growth by about 100% to about 1000% (measured at about 2 days after cell seeding) in cells treated with PDGF in comparison to cells not treated with PDGF. In some embodiments, the cartilage phase is capable of increasing cell number or cell growth by about 100% (measured at about 2 days after cell seeding) in cells treated with PDGF in comparison to cells not treated with PDGF. In some embodiments, the cartilage phase is capable of increasing cell number or cell growth by about 200% (measured at about 2 days after cell seeding) in cells treated with PDGF in comparison to cells not treated with PDGF. In some embodiments, the cartilage phase is capable of increasing cell number or cell growth by about 300% (measured at about 2 days after cell seeding) in cells treated with PDGF in comparison to cells not treated with PDGF. In some embodiments, the cartilage phase is capable of increasing cell number or cell growth by about 400% (measured at about 2 days after cell seeding) in cells treated with PDGF in comparison to cells not treated with PDGF. In some embodiments, the cartilage phase is capable of increasing cell number or cell growth by about 600% (measured at about 2 days after cell seeding) in cells treated with PDGF in comparison to cells not treated with PDGF. In some embodiments, the cartilage phase is capable of increasing cell number or cell growth by about 800% (measured at about 2 days after cell seeding) in cells treated with PDGF in comparison to cells not treated with PDGF. In some embodiments, the cartilage phase is capable of increasing cell number or cell growth by about 1000% (measured at about 2 days after cell seeding) in cells treated with PDGF in comparison to cells not treated with PDGF.

In some embodiments of the present invention, both the osseous phase and the cartilage phase are capable of increasing cell number or cell growth in both phases by about 100% to about 1000% (measured at about 2 days after cell seeding) in cells treated with PDGF in comparison to cells not treated with PDGF. In some embodiments, both the osseous phase and the cartilage phase are capable of increasing cell number or cell growth in both phases by about 100% (measured at about 2 days after cell seeding) in cells treated with PDGF in comparison to cells not treated with PDGF. In some embodiments, both the osseous phase and the cartilage phase are capable of increasing cell number or cell growth in both phases by about 200% (measured at about 2 days after cell seeding) in cells treated with PDGF in comparison to cells not treated with PDGF. In some embodiments, both the osseous phase and the cartilage phase are capable of increasing cell number or cell growth in both phases by about 300% (measured at about 2 days after cell seeding) in cells treated with PDGF in comparison to cells not treated with PDGF. In some embodiments, both the osseous phase and the cartilage phase are capable of increasing cell number or cell growth in both phases by about 400% (measured at about 2 days after cell seeding) in cells treated with PDGF in comparison to cells not treated with PDGF. In some embodiments, both the osseous phase and the cartilage phase are capable of increasing cell number or cell growth in both phases by about 600% (measured at about 2 days after cell seeding) in cells treated with PDGF in comparison to cells not treated with PDGF. In some embodiments, both the osseous phase and the cartilage phase are capable of increasing cell number or cell growth in both phases by about 800% (measured at about 2 days after cell seeding) in cells treated with PDGF in comparison to cells not treated with PDGF. In some embodiments, both the osseous phase and the cartilage phase are capable of increasing cell number or cell growth by about 1000% (measured at about 2 days after cell seeding) in cells treated with PDGF in comparison to cells not treated with PDGF.

In various embodiments, the weight/weight ratio between the osseous phase and the cartilage phase in a scaffolding matrix of a biphasic biocompatible matrix is between about 65:35 to about 99:1. In some embodiments, the weight/weight ratio between the osseous phase and the cartilage phase in a scaffolding matrix of a biphasic biocompatible matrix is about 65:35, about 70:30, about 75:25, about 80:20, about 85:15, about 90:10, about 95:5, about 96:4, about 97:3, about 98:2, or about 99:1.

In some embodiments, the osseous phase is on the bottom of the biphasic biocompatible matrix, and the cartilage phase is on the top of the biphasic biocompatible matrix.

The biphasic biocompatible matrix, according to some embodiments, can be provided in a shape suitable for implantation (e.g., a sphere, a cylinder, or a block). In some embodiments, the biphasic biocompatible matrix can be gels, particles, powders, patches, pads, sheets, plugs, or sponges. For example, when a biphasic biocompatible matrix comprising a scaffolding matrix with an osseous phase and a cartilage phase in the form of a plug is introduced into at least one site of an osteochondral defect (e.g., the bone adjacent to the cartilage, the cartilage, an interface between the cartilage and the bone adjacent to the cartilage, or combinations thereof), the biphasic biocompatible matrix plug is cell compatible and allows for a formulation of the product that yields an implantable plug that assists with the regeneration of both the cartilage (e.g., an articular cartilage, a fibrocartilage, or an elastic cartilage) and the bone (e.g., a subchondral bone or a cancellous bone). Commercially available biphasic biocompatible matrices may be obtained from a variety of sources, including Orthomimetics (e.g., Chondromimetic or RIVERSIDE®; Cambridge, UK), Smith and Nephew (London, UK), and Kensey Nash (OSSEOFIT™, Exton, Pa.). In some embodiments, the biphasic biocompatible matrix is Chondromimetic. In other embodiments, the biphasic biocompatible matrix is not Chondromimetic. In some embodiments, the biphasic biocompatible matrix is OSSEOFIT™. In some embodiments, the biphasic biocompatible matrix is not OSSEOFIT™.

In some embodiments, the biphasic biocompatible matrix is moldable, extrudable, and/or injectable. Moldable biphasic biocompatible matrices can facilitate efficient placement of compositions of the present invention in and around a cartilage (e.g., an articular cartilage, a fibrocartilage, and an elastic cartilage) and a bone (e.g., a subchondral bone or a cancellous bone). In some embodiments, the biphasic biocompatible matrix is applied to a bone adjacent to a cartilage, a cartilage, and the interface between the bone and the cartilage with a spatula or equivalent device. In some embodiments, the biphasic biocompatible matrix is flowable. The flowable biphasic biocompatible matrix, in some embodiments, can be applied to at least one site of an osteochondral defect (e.g., the bone adjacent to the cartilage, the cartilage, an interface between the cartilage and the bone adjacent to the cartilage, or combinations thereof)) through a syringe and needle or cannula. In some embodiments, the flowable biphasic biocompatible matrix can be applied to a surgically exposed site of at least one site of an osteochondral defect (e.g., the bone adjacent to the cartilage, the cartilage, an interface between the cartilage and the bone adjacent to the cartilage, or combinations thereof). In some embodiments, the biphasic biocompatible matrix is in a plug form and can be “press fit” into the osteochondral lesion.

In some embodiments, the biphasic biocompatible matrix comprises a scaffolding material. The scaffolding material forms a porous structure comprising an osseous phase and a cartilage phase, which allows for PDGF to be released from the biphasic biocompatible matrix. In some embodiments, the biphasic biocompatible matrix comprises a 5% collagen in both the osseous phase and the cartilage phase which allows for a higher percentage of PDGF to be released in comparison to a 6% collagen or a 7% collagen. In some embodiments, the biphasic biocompatible matrix comprising pores with porosity greater than about 85% allows for a higher percentage of PDGF to be released in comparison to a biphasic biocompatible matrix with porosity lower than about 85%. In some embodiments, the biphasic biocompatible matrix comprising pores with porosity greater than about 90% allows for a higher percentage of PDGF to be released in comparison to a biphasic biocompatible matrix with porosity lower than about 90%.

In some embodiments, the biphasic biocompatible matrix allows for release of PDGF at 24 hours. In some embodiments, the biphasic biocompatible matrix allows for release of at least about 50% of PDGF at 24 hrs. In some embodiments, the biphasic biocompatible matrix allows for release of at least about 55% of PDGF at 24 hrs. In some embodiments, the biphasic biocompatible matrix allows for release of at least about 60% of PDGF at 24 hrs. In some embodiments, the biphasic biocompatible matrix allows for release of at least about 65% of PDGF at 24 hrs. In some embodiments, the biphasic biocompatible matrix allows for release of at least about 70% of PDGF at 24 hrs. In some embodiments, the biphasic biocompatible matrix allows for release of at least about 71% of PDGF at 24 hrs. In some embodiments, the biphasic biocompatible matrix allows for release of at least about 72% of PDGF at 24 hrs. In some embodiments, the biphasic biocompatible matrix allows for release of at least about 73% of PDGF at 24 hrs. In some embodiments, the biphasic biocompatible matrix allows for release of at least about 74% of PDGF at 24 hrs. In some embodiments, the biphasic biocompatible matrix allows for release of at least about 75% of PDGF at 24 hrs. In some embodiments, the biphasic biocompatible matrix allows for release of at least about 80% of PDGF at 24 hrs. In some embodiments, the biphasic biocompatible matrix allows for release of at least about 85% of PDGF at 24 hrs. In some embodiments, the biphasic biocompatible matrix allows for release of at least about 90% of PDGF at 24 hrs. In some embodiments, the biphasic biocompatible matrix allows for release of at least about 95% of PDGF at 24 hrs. The PDGF released or eluted from the scaffolding material may be biochemically stable.

In some embodiments, the biphasic biocompatible matrix allows for release of at least about 75,000 ng of PDGF at 24 hrs. In some embodiments, the biphasic biocompatible matrix allows for release of at least about 80,000 ng of PDGF at 24 hrs. In some embodiments, the biphasic biocompatible matrix allows for release of at least about 81,000 ng of PDGF at 24 hrs. In some embodiments, the biphasic biocompatible matrix allows for release of at least about 82,000 ng of PDGF at 24 hrs. In some embodiments, the biphasic biocompatible matrix allows for release of at least about 83,000 ng of PDGF at 24 hrs. In some embodiments, the biphasic biocompatible matrix allows for release of at least about 84,000 ng of PDGF at 24 hrs. In some embodiments, the biphasic biocompatible matrix allows for release of at least about 85,000 ng of PDGF at 24 hrs. In some embodiments, the biphasic biocompatible matrix allows for release of at least about 86,000 ng of PDGF at 24 hrs. In some embodiments, the biphasic biocompatible matrix allows for release of at least about 87,000 ng of PDGF at 24 hrs. In some embodiments, the biphasic biocompatible matrix allows for release of at least about 88,000 ng of PDGF at 24 hrs. In some embodiments, the biphasic biocompatible matrix allows for release of at least about 89,000 ng of PDGF at 24 hrs. In some embodiments, the biphasic biocompatible matrix allows for release of at least about 90,000 ng of PDGF at 24 hrs. The PDGF released or eluted from the scaffolding material may be biochemically stable.

In some embodiments of the present invention, the maximum gross score by area is increased by about 100% to about 500% (measured at 12 weeks after administration of the matrix) in an individual treated with a composition comprising a biphasic biocompatible matrix and PDGF in comparison to an individual treated with a composition comprising the biphasic biocompatible matrix alone. In some embodiments, the maximum gross score by area is increased by about 100% (measured at 12 weeks after administration of the matrix) in an individual treated with a composition comprising a biphasic biocompatible matrix and PDGF in comparison to an individual treated with a composition comprising the biphasic biocompatible matrix alone. In some embodiments, the maximum gross score by area is increased by about 200% (measured at 12 weeks after administration of the matrix) in an individual treated with a composition comprising a biphasic biocompatible matrix and PDGF in comparison to an individual treated with a composition comprising the biphasic biocompatible matrix alone. In some embodiments, the maximum gross score by area is increased by about 300% (measured at 12 weeks after administration of the matrix) in an individual treated with a composition comprising a biphasic biocompatible matrix and PDGF in comparison to an individual treated with a composition comprising the biphasic biocompatible matrix alone. In some embodiments, the maximum gross score by area is increased by about 400% (measured at 12 weeks after administration of the matrix) in an individual treated with a composition comprising a biphasic biocompatible matrix and PDGF in comparison to an individual treated with a composition comprising the biphasic biocompatible matrix alone. In some embodiments, the maximum gross score by area is increased by about 500% (measured at 12 weeks after administration of the matrix) in an individual treated with a composition comprising a biphasic biocompatible matrix and PDGF in comparison to an individual treated with a composition comprising the biphasic biocompatible matrix alone

In some embodiments, the biphasic biocompatible matrix allows for infiltration of cells into pores of the matrix. In some embodiments, the scaffolding material comprising an osseous phase and a cartilage phase in a biphasic biocompatible matrix allows for infiltration of cells into pores of the matrix. In some embodiments, the biphasic biocompatible matrix allows for attachment of cells. In some embodiments, the scaffolding material comprising an osseous phase and a cartilage phase in a biphasic biocompatible matrix allows for attachment of cells. In some embodiments, the infiltrating or attached cells are chondrocytes. In some embodiments, the infiltrating or attached cells are mesenchymal stem cells (or marrow stromal cells). In some embodiments, the infiltrating cells are osteoblasts.

In some embodiments, the biphasic biocompatible matrix is porous and operable to absorb water or other fluid. In some embodiments, the scaffolding material comprising an osseous phase and a cartilage phase in a biphasic biocompatible matrix is porous and operable to absorb water or other fluid in an amount ranging from about 1× to about 15× the mass of the biphasic biocompatible matrix. In some embodiments, a complete absorption of a biphasic biocompatible matrix can be achieved with about 300 μl to about 1,000 μl of water, a buffer, or other fluid. In some embodiments, a complete absorption of a biphasic biocompatible matrix can be achieved with about 300 μl, about 350 μl, about 400 μl, about 450 μl, about 500 μl, about 550 μl, about 600 μl, about 650 μl, about 700 μl, about 750 μl, about 800 μl, about 850 μl, about 900 μl, about 950 μl, or about 1,000 μl of water, a buffer, or other fluid. A buffer can be, for example, an elution buffer of varying salt concentrations.

In some embodiments, the biphasic biocompatible matrix comprises a porous structure having multidirectional and/or interconnected pores. In some embodiments, the scaffolding material comprising an osseous phase and a cartilage phase in a biphasic biocompatible matrix comprises a porous structure having multidirectional and/or interconnected pores. Porous structure, according to some embodiments, comprises pores having diameters ranging from about 1 μm to about 1 mm. In some embodiment, the biphasic biocompatible matrix comprises macropores having diameters ranging from about 100 μm to about 1 mm. In some embodiments, the biphasic biocompatible matrix comprises mesopores having diameters ranging from about 10 μm to about 100 μm. In some embodiments, the biphasic biocompatible matrix comprises micropores having diameters less than about 10 μm. Various embodiments of the present invention contemplate a biphasic biocompatible matrix comprising macropores, mesopores, micropores or any combination thereof.

In some embodiments, the scaffolding material comprising an osseous phase and a cartilage phase in a biphasic biocompatible matrix comprises a porous structure having pores that are not interconnected. In some embodiments, the scaffolding material comprising an osseous phase and a cartilage phase in a biphasic biocompatible matrix comprises a porous structure having pores that are interconnected. In some embodiments, the scaffolding material comprising an osseous phase and a cartilage phase in a biphasic biocompatible matrix comprises a porous structure having a mixture of interconnected pores and pores that are not interconnected.

In some embodiments, the osseous phase in a scaffolding material comprises a porous structure having pores that are not interconnected. In some embodiments, the osseous phase in a scaffolding material comprises a porous structure having pores that are interconnected. In some embodiments, the osseous phase in a scaffolding material comprises a porous structure having a mixture of interconnected pores and pores that are not interconnected.

In some embodiments, the cartilage phase in a scaffolding material comprises a porous structure having pores that are not interconnected. In some embodiments, the cartilage phase in a scaffolding material comprises a porous structure having pores that are interconnected. In some embodiments, the cartilage phase in a scaffolding material comprises a porous structure having a mixture of interconnected pores and pores that are not interconnected.

In some embodiments, the biphasic biocompatible matrix can be resorbed within about one year of in vivo administration. In some embodiments, the biphasic biocompatible matrix can be resorbed within about 1, 2, 3 4, 5, 6, 7, 8, 9, 10, or 11 months of in vivo administration. In some embodiments, the biphasic biocompatible matrix can be resorbed within about 30 days of in vivo administration. In some embodiments, the biphasic biocompatible matrix can be resorbed within about 10-14 days of in vivo administration. In some embodiments, the biphasic biocompatible matrix can be resorbed within about 10 days of in vivo administration. In some embodiments, the biphasic biocompatible matrix is resorbed such that at least about 70% to about 95% of the matrix is resorbed. In some embodiments, the biphasic biocompatible matrix is resorbed such that at least about 80% of the matrix is resorbed. Bioresorbability is dependent on: (1) the nature of the biphasic biocompatible matrix material (i.e., its chemical make up, physical structure and size); (2) the location within the body in which the biphasic biocompatible matrix is placed; (3) the amount of biphasic biocompatible matrix material that is used; (4) the metabolic state of the patient (diabetic/non-diabetic, osteoporotic, smoker, old age, steroid use, etc.); (5) the extent and/or type of injury treated; and (6) the use of other materials in addition to the biphasic biocompatible matrix such as other bone anabolic, catabolic and anti-catabolic factors.

In some embodiments, the scaffolding material comprising an osseous phase and a cartilage phase in a biphasic biocompatible matrix can be resorbed within about one year of in vivo administration. In some embodiments, the scaffolding material can be resorbed within about 1, 2, 3 4, 5, 6, 7, 8, 9, 10, or 11 months of in vivo administration. In some embodiments, the scaffolding material can be resorbed within about 30 days of in vivo administration. In some embodiments, the scaffolding material can be resorbed within about 10-14 days of in vivo administration. In some embodiments, the scaffolding material can be resorbed within about 10 days of in vivo administration. In some embodiments, the scaffolding material is resorbed such that at least about 70% to about 95% of the material is resorbed. In some embodiments, the scaffolding material is resorbed such that at least about 80% of the matrix is resorbed.

### Biocompatible Binder

In some embodiments, the biphasic biocompatible matrix comprises a scaffolding matrix and a biocompatible binder. Biocompatible binders can comprise one or more materials operable to promote cohesion between one or more substances. A biocompatible binder, for example, can promote adhesion between particles of a scaffolding material in the formation of a biphasic biocompatible matrix. In certain embodiments, the same material may serve as both a scaffolding material and a binder if such material acts to promote cohesion between the substances and provides a framework for new cartilage and bone growth to occur. See WO2008/005427 and U.S. Ser. No. 11/772,646 (U.S. Publication 2008/00274470), hereby incorporated by reference in their entirety.

Biocompatible binders, in some embodiments, can comprise one or more of: collagen, elastin, polysaccharides, nucleic acids, carbohydrates, proteins, polypeptides, poly(α-hydroxy acids), poly(lactones), poly(amino acids), poly(anhydrides), polyurethanes, poly(orthoesters), poly(anhydride-co-imides), poly(orthocarbonates), poly(α-hydroxy alkanoates), poly(dioxanones), poly(phosphoesters), polylactic acid (PLA), poly(L-lactide) (PLLA), poly(D,L-lactide) (PDLLA), polyglycolide or polyglycolic acid (PGA), poly(lactide-co-glycolide (PLGA), poly(L-lactide-co-D,L-lactide), poly(D,L-lactide-co-trimethylene carbonate), polyhydroxybutyrate (PHB), poly(ε-caprolactone), poly(δ-valerolactone), poly(γ-butyrolactone), poly(caprolactone), polyacrylic acid, polycarboxylic acid, poly(allylamine hydrochloride), poly(diallyldimethylammonium chloride), poly(ethyleneimine), polypropylene fumarate, polyvinyl alcohol, polyvinylpyrrolidone, polyethylene, polymethylmethacrylate, carbon fibers, poly(ethylene glycol), poly(ethylene oxide), poly(vinyl alcohol), poly(vinylpyrrolidone), poly(ethyloxazoline), poly(ethylene oxide)-co-poly(propylene oxide) block copolymers, poly(ethylene terephthalate)polyamide, copolymers, and mixtures thereof.

Biocompatible binders, in some embodiments, can comprise one or more of: alginic acid, arabic gum, guar gum, xantham gum, gelatin, chitin, chitosan, chitosan acetate, chitosan lactate, chondroitin sulfate, N,β-carboxymethyl chitosan, a dextran (e.g., α-cyclodextrin, β-cyclodextrin, γ-cyclodextrin, or sodium dextran sulfate), fibrin glue, lecithin, phosphatidylcholine derivatives, glycerol, hyaluronic acid, sodium hyaluronate, a cellulose (e.g., methylcellulose, carboxymethylcellulose, hydroxypropyl methylcellulose, or hydroxyethyl cellulose), a glucosamine, a proteoglycan, a starch (e.g., hydroxyethyl starch or starch soluble), lactic acid, a pluronic acids, sodium glycerophosphate, glycogen, a keratin, silk, and derivatives and mixtures thereof.

In some embodiments, the biocompatible binder is water-soluble. A water-soluble binder can dissolve from the biphasic biocompatible matrix shortly after its implantation, thereby introducing macroporosity into the biocompatible matrix. Macroporosity, as discussed herein, can increase the osteoconductivity of the implant material by enhancing the access and, consequently, the remodeling activity of the osteoclasts and osteoblasts at the implant site.

In some embodiments, the biocompatible binder can be present in a biphasic biocompatible matrix in an amount ranging from about 5 weight percent to about 50 weight percent of the matrix. In some embodiments, the biocompatible binder can be present in an amount ranging from about 10 weight percent to about 40 weight percent of the biphasic biocompatible matrix. In some embodiments, the biocompatible binder can be present in an amount ranging from about 15 weight percent to about 35 weight percent of the biphasic biocompatible matrix. In some embodiments, the biocompatible binder can be present in an amount of about 20 weight percent of the biphasic biocompatible matrix. In some embodiments, the biocompatible binder can be present in an amount of less than about 50 weight percent of the biphasic biocompatible matrix. In some embodiments, the biocompatible binder can be present in an amount of less than about 40 weight percent of the biphasic biocompatible matrix. In some embodiments, the biocompatible binder can be present in an amount of less than about 30 weight percent of the biphasic biocompatible matrix. In some embodiments, the biocompatible binder can be present in an amount of less than about 20 weight percent of the biphasic biocompatible matrix. In some embodiments, the biocompatible binder can be present in an amount of less than about 10 weight percent of the biphasic biocompatible matrix. In some embodiments, the biocompatible binder can be present in an amount of less than about 5 weight percent of the biphasic biocompatible matrix.

A biphasic biocompatible matrix comprising a scaffolding material and optionally a biocompatible binder, according to some embodiments, can be flowable, moldable, and/or extrudable. In some embodiments, the biphasic biocompatible matrix can be in the form of a paste or putty. The biocompatible matrix in the form of a paste or putty, in some embodiments, can comprise particles of a scaffolding material adhered to one another by a biocompatible binder.

A biphasic biocompatible matrix in paste or putty form can be molded into the desired implant shape or can be molded to the contours of the implantation site. In some embodiments, the biphasic biocompatible matrix in paste or putty form can be injected into an implantation site with a syringe or cannula. In some embodiments, moldable and/or flowable scaffolding materials can be applied to at least one site of the osteochondral defect in a bone adjacent to a cartilage (e.g., the bone adjacent to the cartilage, the cartilage, an interface between the cartilage and the bone adjacent to the cartilage, or combinations thereof).

In some embodiments, the biphasic biocompatible matrix in paste or putty form does not harden and retains a flowable and moldable form subsequent to implantation. In some embodiments, a paste or putty can harden subsequent to implantation, thereby reducing matrix flowability and moldability.

A biphasic biocompatible matrix comprising a scaffolding material and an optional biocompatible binder, in some embodiments, can also be provided in a predetermined shape including a block, sphere, or cylinder or any desired shape, for example, a shape defined by a mold or a site of application. In some embodiments, the biphasic biocompatible matrix comprising a scaffolding material and an optional biocompatible binder can be provided in the form of gels, particles, powders, sheets, patches, pads, plugs, or sponges.

A biphasic biocompatible matrix comprising a scaffolding material and an optional biocompatible binder, in some embodiments, is bioresorbable. The biphasic biocompatible matrix, in some embodiments, can be resorbed within about one year of in vivo implantation. In some embodiments, the biphasic biocompatible matrix comprising a scaffolding material and an optional biocompatible binder can be resorbed within about 1, 2, 3, 4, 5, 6, 7, 8, 9, 10, or 11 months of in vivo implantation. In some embodiments, the biphasic biocompatible matrix comprising a scaffolding material and an optional biocompatible binder can be resorbed within about 30 days of in vivo administration. In some embodiments, the biphasic biocompatible matrix comprising a scaffolding material and an optional biocompatible binder can be resorbed within about 10-14 days of in vivo administration. In some embodiments, the biphasic biocompatible matrix comprising a scaffolding material and an optional biocompatible binder can be resorbed within about 10 days of in vivo administration. In some embodiments, the biphasic biocompatible matrix comprising a scaffolding material and an optional biocompatible binder is resorbed such that at least about 70% to about 95% of the matrix is resorbed. In some embodiments, the biphasic biocompatible matrix comprising a scaffolding material and an optional biocompatible binder is resorbed such that at least about 80% of the matrix is resorbed.

### Platelet-Derived Growth Factor

The invention provides for compositions and methods for treating an osteochondral defect in a cartilage and a bone. In some embodiments, provided are compositions and methods for treating an osteochondral defect in an cartilage and a bone adjacent to the cartilage in an individual. In some embodiments, the cartilage comprises an articular cartilage, a fibrocartilage, or an elastic cartilage. In some embodiments, the bone comprises a subchondral bone or a cancellous bone.

A biphasic biocompatible matrix, according to some embodiments of the present invention, comprises a scaffolding material and PDGF. A scaffolding material may further comprise an osseous phase and a cartilage phase. PDGF is a growth factor released from platelets at sites of injury. PDGF synergizes with Vascular Endothelial Growth Factor (VEGF) to promote angiogenesis (revascularization) and stimulate chemotaxis and proliferation of mesenchymally-derived cells including tenocytes, osteoblasts, chondrocytes, and vascular smooth muscle cells. When, together with a biphasic biocompatible matrix, introduced into at least one site of an osteochondral defect (e.g., the bone adjacent to the cartilage, the cartilage, an interface between the cartilage and the bone adjacent to the cartilage, or combinations thereof). PDGF evokes the synthesis of Type II collagen (the primary collagen subtype of hyaline cartilage), increases the recruitment of adequate number of stem cells, and enhances both the bony ingrowth and the cartilage regeneration in osteochondral defects.

In one aspect, compositions and methods provided by the present invention may comprise a biphasic biocompatible matrix and a solution of PDGF, wherein the solution is dispersed in the biocompatible matrix. In various embodiments, PDGF is present in a solution and is at a concentration in the range of about 0.01 mg/ml to about 10.0 mg/ml. In some embodiments, PDGF is present in a solution and is at a concentration in the range of about 0.01 mg/ml to about 1.0 mg/ml. In some embodiments, PDGF is present in a solution and is at a concentration in the range of about 0.01 mg/ml to about 2.0 mg/ml. In some embodiments, PDGF is present in a solution and is at a concentration in the range of about 0.01 mg/ml to about 3.0 mg/ml. In some embodiments, PDGF is present in a solution and is at a concentration in the range of about 0.05 mg/ml to about 5.0 mg/ml. In some embodiments, PDGF is present in a solution and is at a concentration in the range of about 0.1 mg/ml to about 5.0 mg/ml. In some embodiments, PDGF is present in a solution and is at a concentration in the range of about 0.1 mg/ml to about 3.0 mg/ml. In some embodiments, PDGF is at a concentration in the range of about 0.1 mg/ml to about 1.0 mg/ml. In some embodiments, PDGF is at a concentration of about 0.03 mg/ml, about 0.15 mg/ml, about 0.3 mg/ml, or about 1.0 mg/ml. In some embodiments, PDGF is present in the solution at any one of the following concentrations: about 0.05 mg/ml; about 0.1 mg/ml; about 0.2 mg/ml; about 0.25 mg/ml; about 0.35 mg/ml; about 0.4 mg/ml; about 0.45 mg/ml; about 0.5 mg/ml, about 0.55 mg/ml, about 0.6 mg/ml, about 0.65 mg/ml, about 0.7 mg/ml; about 0.75 mg/ml; about 0.8 mg/ml; about 0.85 mg/ml; about 0.9 mg/ml; about 0.95 mg/ml; about 1.5 mg/ml, or about 2.0 mg/ml. It is to be understood that these concentrations are simply examples of particular embodiments, and that the concentration of PDGF may be within any of the concentration ranges stated above.

In one variation, compositions and methods provided by the present invention may comprise a biphasic biocompatible matrix and a solution of PDGF, wherein the PDGF solution is lyophilized or freeze-dried into the biphasic biocompatible matrix. The composition can be reconstituted for use in methods described herein.

Various amounts of PDGF may be used in the compositions of the present invention. Examples of amounts of PDGF that may be used include amounts in the following ranges: about 1 μg to about 50 mg, about 1 μg to about 10 mg, about 1 μg to about 1 mg, about 1 μg to about 500 μg, about 10 μg to about 25 mg, about 10 μg to about 500 μg, about 100 μg to about 10 mg, or about 250 μg to about 5 mg. In some embodiments, PDGF is at an amount of about 15 μg, about 75 μg, about 150 μg, or about 500 μg.

The concentration of PDGF (or other growth factors) in some embodiments of the present invention can be determined by using an enzyme-linked immunoassay as described in U.S. Pat. Nos. 6,221,625; 5,747,273; and 5,290,708, or any other assay known in the art for determining PDGF concentration. When provided herein, the molar concentration of PDGF is determined based on the molecular weight of PDGF dimer (e.g., PDGF-BB, MW about 25 kDa).

PDGF may comprise PDGF homodimers and/or heterodimers, including PDGF-AA, PDGF-BB, PDGF-AB, PDGF-CC, PDGF-DD, and mixtures and derivatives thereof. In some embodiments, PDGF comprises PDGF-BB. In some embodiments, PDGF comprises a recombinant human PDGF, such as rhPDGF-BB.

In some embodiments, PDGF can be obtained from natural sources. In some embodiments, PDGF can be produced by recombinant DNA techniques. In some embodiments, PDGF or fragments thereof may be produced using peptide synthesis techniques known to one of skill in the art, such as solid phase peptide synthesis.

When obtained from natural sources, PDGF can be derived from biological fluids. Biological fluids, according to some embodiments, can comprise any treated or untreated fluid associated with living organisms including blood. Biological fluids can also comprise blood components including platelet concentrate, apheresed platelets, platelet-rich plasma, plasma, serum, fresh frozen plasma, and buffy coat. Biological fluids can comprise platelets separated from plasma and resuspended in a physiological fluid.

When produced by recombinant DNA techniques, a DNA sequence encoding a single monomer (e.g., PDGF B-chain or A-chain) can be inserted into cultured prokaryotic or eukaryotic cells for expression to subsequently produce the homodimer (e.g., PDGF-BB or PDGF-AA). The homodimer PDGF produced by recombinant techniques may be used in some embodiments. In some embodiments, a PDGF heterodimer can be generated by inserting DNA sequences encoding for both monomeric units of the heterodimer into cultured prokaryotic or eukaryotic cells and allowing the translated monomeric units to be processed by the cells to produce the heterodimer (e.g., PDGF-AB). Commercially available recombinant human PDGF-BB may be obtained from a variety of sources, including cGAMP recombinant PDGF-BB from Chiron/Norvartis Corporation (Emeryville, Calif.), research grade rhPDGF-BB (R&D Systems, Inc. (Minneapolis, Minn.), BD Biosciences (San Jose, Calif.), and Chemicon, International (Temecula, Calif.)).

In some embodiments of the present invention, PDGF comprises one or more PDGF fragments. In some embodiments, rhPDGF-B comprises one or more of the following fragments: amino acid sequences 1-31, 1-32, 33-108, 33-109, and/or 1-108 of the entire B chain. The complete amino acid sequence (AA 1-109) of the B chain of PDGF is provided in FIG. 15 of U.S. Pat. No. 5,516,896. It is to be understood that the rhPDGF compositions of the present invention may comprise a combination of intact rhPDGF-B (AA 1-109) and fragments thereof. Other fragments of PDGF may be employed such as those disclosed in U.S. Pat. No. 5,516,896. In accordance with some embodiments, the rhPDGF-BB comprises at least 65% of intact rhPDGF-B (AA 1-109). In accordance with some embodiments, the rhPDGF-BB comprises at least 75%, 80%, 85%, 90%, 95%, or 99% of intact rhPDGF-B (AA 1-109).

In some embodiments of the present invention, PDGF can be in a purified form. Purified PDGF, as used herein, comprises compositions having greater than about 95% by weight PDGF prior to incorporation in solutions of the present invention. The solution may be prepared using any pharmaceutically acceptable buffer or diluent. In some embodiments, the PDGF can be substantially purified. Substantially purified PDGF, as used herein, comprises compositions having about 5% to about 95% by weight PDGF prior to incorporation into solutions of the present invention. In one embodiment, substantially purified PDGF comprises compositions having about 65% to about 95% by weight PDGF prior to incorporation into solutions of the present invention. In some embodiments, substantially purified PDGF comprises compositions having about 70% to about 95%, about 75% to about 95%, about 80% to about 95%, about 85% to about 95%, or about 90% to about 95%, by weight PDGF, prior to incorporation into solutions of the present invention. Purified PDGF and substantially purified PDGF may be incorporated into the scaffolding matrix.

In a further embodiment, PDGF can be partially purified. Partially purified PDGF, as used herein, comprises compositions having PDGF in the context of platelet-rich plasma, fresh frozen plasma, or any other blood product that requires collection and separation to produce PDGF. Embodiments of the present invention contemplate that any of the PDGF isoforms provided herein, including homodimers and heterodimers, can be purified or partially purified. Compositions of the present invention comprising PDGF mixtures may comprise PDGF isoforms or PDGF fragments in partially purified proportions. Partially purified and purified PDGF, in some embodiments, can be prepared as described in U.S. Ser. No. 11/159,533 (U.S. Publication 20060084602).

In some embodiments, solutions comprising PDGF are formed by solubilizing PDGF in one or more buffers. Buffers suitable for use in PDGF solutions of the present invention can comprise, but are not limited to, carbonates, phosphates (e.g., phosphate-buffered saline), histidine, acetates (e.g., sodium acetate), acidic buffers such as acetic acid and HCl, and organic buffers such as lysine, Tris buffers (e.g., tris(hydroxymethyl)aminoethane), N-2-hydroxyethylpiperazine-N′-2-ethanesulfonic acid (HEPES), and 3-(N-morpholino) propanesulfonic acid (MOPS). Buffers can be selected based on biocompatibility with PDGF and the buffer's ability to impede undesirable protein modification. Buffers can additionally be selected based on compatibility with host tissues. In one embodiment, sodium acetate buffer is used. The buffers may be employed at different molarities, for example about 0.1 mM to about 100 mM, about 1 mM to about 50 mM, about 5 mM to about 40 mM, about 10 mM to about 30 mM, or about 15 mM to about 25 mM, or any molarity within these ranges. In some embodiments, an acetate buffer is employed at a molarity of about 20 mM.

In another embodiment, solutions comprising PDGF may be formed by solubilizing lyophilized PDGF in water, wherein prior to solubilization the PDGF is lyophilized from an appropriate buffer.

Solutions comprising PDGF, according to embodiments of the present invention, can have a pH ranging from about 3.0 to about 8.0. In one embodiment, a solution comprising PDGF has a pH ranging from about 5.0 to about 8.0, more preferably about 5.5 to about 7.0, most preferably about 5.5 to about 6.5, or any value within these ranges. The pH of solutions comprising PDGF, in some embodiments, can be compatible with the prolonged stability and efficacy of PDGF or any other desired biologically active agent. PDGF is generally more stable in an acidic environment. Therefore, in accordance with some embodiments, the present invention comprises an acidic storage formulation of a PDGF solution. In accordance with some embodiments, the PDGF solution preferably has a pH from about 3.0 to about 7.0, and more preferably from about 4.0 to about 6.5. The biological activity of PDGF, however, can be optimized in a solution having a neutral pH range. Therefore, in some embodiments, the present invention comprises a neutral pH formulation of a PDGF solution. In accordance with this embodiment, the PDGF solution preferably has a pH from about 5.0 to about 8.0, more preferably about 5.5 to about 7.0, most preferably about 5.5 to about 6.5.

In some embodiments, the pH of the PDGF-containing solution may be altered to optimize the binding kinetics of PDGF to a matrix substrate. If desired, as the pH of the material equilibrates to adjacent material, the bound PDGF may become labile.

The pH of solutions comprising PDGF, in some embodiments, can be controlled by the buffers recited herein. Various proteins demonstrate different pH ranges in which they are stable. Protein stabilities are primarily reflected by isoelectric points and charges on the proteins. The pH range can affect the conformational structure of a protein and the susceptibility of a protein to proteolytic degradation, hydrolysis, oxidation, and other processes that can result in modification to the structure and/or biological activity of the protein.

In some embodiments, solutions comprising PDGF can further comprise additional components, such as other biologically active agents. In some embodiments, solutions comprising PDGF can further comprise cell culture media, other stabilizing proteins such as albumin, antibacterial agents, protease inhibitors (e.g., ethylenediaminetetraacetic acid (EDTA), ethylene glycol-bis(beta-aminoethylether)-N,N,N′,N′-tetraacetic acid (EGTA), aprotinin, E-aminocaproic acid (EACA), etc.) and/or other growth factors such as fibroblast growth factors (FGFs), epidermal growth factors (EGFs), transforming growth factors (TGFs), keratinocyte growth factors (KGFs), insulin-like growth factors (IGEs), bone morphogenetic proteins (BMPs), or other PDGFs including compositions of PDGF-AA, PDGF-BB, PDGF-AB, PDGF-CC and/or PDGF-DD.

In some embodiments, the biphasic biocompatible matrix is capable of absorbing an amount of a solution comprising PDGF that is between a range of about 25% to about 2000% by weight of the biphasic biocompatible matrix. In some embodiments, the biphasic biocompatible matrix is capable of absorbing an amount of a solution comprising PDGF that is between a range of about 100% to about 1600% by weight of the biphasic biocompatible matrix. In some embodiments, the biphasic biocompatible matrix is capable of absorbing an amount of a solution comprising PDGF that is equal to at least about 25% by weight of the biphasic biocompatible matrix. In some embodiments, the biphasic biocompatible matrix is capable of absorbing an amount of a solution comprising PDGF that is equal to at least about 100% by weight of the biphasic biocompatible matrix. In some embodiments, the biphasic biocompatible matrix is capable of absorbing an amount of a solution comprising PDGF that is equal to at least about 500% by weight of the biphasic biocompatible matrix. In some embodiments, the biphasic biocompatible matrix is capable of absorbing an amount of a solution comprising PDGF that is equal to at least about 1000% by weight of the biphasic biocompatible matrix. In some embodiments, the biphasic biocompatible matrix is capable of absorbing an amount of a solution comprising PDGF that is equal to at least about 1550% by weight of the biphasic biocompatible matrix. In some embodiments, the biphasic biocompatible matrix is capable of absorbing an amount of a solution comprising PDGF that is equal to at least about 1600% by weight of the biphasic biocompatible matrix. In some embodiments, the biphasic biocompatible matrix is capable of absorbing an amount of a solution comprising PDGF that is equal to at least about 2000% by weight of the biphasic biocompatible matrix.

### Compositions Further Comprising Biologically Active Agents

Compositions and methods of the present invention, according to some embodiments, can further comprise one or more biologically active agents in addition to PDGF. Biologically active agents that can be incorporated into compositions of the present invention, in addition to PDGF, can comprise, for example, organic molecules, inorganic materials, proteins, peptides, nucleic acids (e.g., genes, gene fragments, small-interfering ribonucleic acids (siRNAs), gene regulatory sequences, nuclear transcriptional factors and antisense molecules), nucleoproteins, polysaccharides (e.g., heparin), glycoproteins, and lipoproteins. Non-limiting examples of biologically active compounds that can be incorporated into compositions of the present invention, including, e.g., anti-cancer agents, antibiotics, analgesics, anti-inflammatory agents, immunosuppressants, enzyme inhibitors, antihistamines, hormones, muscle relaxants, prostaglandins, trophic factors, osteoinductive proteins, growth factors, and vaccines, are disclosed in U.S. Ser. No. 11/159,533 (U.S. Publication 20060084602). Biologically active compounds that can be incorporated into compositions of the present invention, in some embodiments, include osteoinductive factors such as insulin-like growth factors, fibroblast growth factors, or other PDGFs. In accordance with some embodiments, biologically active compounds that can be incorporated into compositions of the present invention preferably include osteoinductive and osteostimulatory factors such as bone morphogenetic proteins (BMPs), BMP mimetics, calcitonin, calcitonin mimetics, statins, statin derivatives, fibroblast growth factors, insulin-like growth factors, growth differentiating factors, and/or parathyroid hormone. Additional factors for incorporation into compositions of the present invention, in some embodiments, include protease inhibitors, as well as osteoporotic treatments that decrease bone resorption including bisphosphonates, and antibodies to the NF-kB (RANK) ligand.

Standard protocols and regimens for delivery of additional biologically active agents are known in the art. Additional biologically active agents can be introduced into compositions of the present invention in amounts that allow delivery of an appropriate dosage of the agent to the at least one site of the osteochondral defect (e.g., the bone adjacent to the cartilage, the cartilage, an interface between the cartilage and the bone adjacent to the cartilage, or combinations thereof). In most cases, dosages are determined using guidelines known to practitioners and applicable to the particular agent in question. The amount of an additional biologically active agent to be included in a composition of the present invention can depend on such variables as the type and extent of the condition, the overall health status of the particular patient, the formulation of the biologically active agent, release kinetics, and the bioresorbability of the biocompatible matrix.

Methods for Treating Defects and/or Injuries to Cartilage

The present invention provides methods for treating osteochondral defects in a cartilage and a bone. In one aspect, methods for treating osteochondral defects in a cartilage and a bone may comprise providing a composition comprising a PDGF solution disposed in a biphasic biocompatible matrix and applying the composition to at least one site of an osteochondral defect. In some embodiments, the PDGF solution is disposed within the osseous and/or cartilage phase(s).

In another aspect, methods for treating osteochondral defects in a cartilage and a bone may comprise providing a composition comprising lyophilized or freeze-dried PDGF from a PDGF solution with predetermined concentration in a biphasic biocompatible matrix, hydrating the composition with normal saline solution or water to at least one site of an osteochondral defect, and applying the composition to the same site(s).

In some embodiments, the method for treating an osteochondral defect in a cartilage and a bone adjacent to the cartilage in an individual comprises administering to the individual an effective amount of a composition comprising a biphasic biocompatible matrix and PDGF to at least one site of the osteochondral defect, wherein the biphasic biocompatible matrix comprises a scaffolding material and wherein the scaffolding material forms a porous structure comprising an osseous phase and a cartilage phase.

In some embodiments, the bone comprises a subchondral bone or a cancellous bone. In some embodiments, the cartilage comprises an articular cartilage, a fibrocartilage, or an elastic cartilage.

In some embodiments, articular cartilage comprises articular cartilage of the knee, including that of the femur and/or tibia. In some embodiments, the articular cartilage comprises femoral condyle or trochlear. In some embodiments, the articular cartilage comprises articular cartilage of the glenohumeral joint, elbow and radioulnar joints, interphalangeal joint, talus (e.g., foot and ankle), and/or hip.

In some embodiments, a composition comprising a PDGF solution disposed in a biphasic biocompatible matrix can be applied through affixing a combination of staples, tacks, and fibrin glue to the perforated subchondral bone surface and inserting the composition into both the articular cartilage and the subchondral bone or cancellous bone.

In some embodiments of the present invention, the method may be performed using open or mini-open arthroscopic techniques, endoscopic techniques, laparoscopic techniques, or any other suitable minimally-invasive techniques.

In some embodiments, the composition comprising a PDGF solution disposed in a biphasic biocompatible matrix can be applied with the aid of a delivery device. For example, the delivery device comprises an outer sleeve, which can be used to load the composition into a site of an osteochondral defect in a cartilage and a bone adjacent to the cartilage. In some embodiments, a site of an osteochondral defect comprises the bone adjacent to the cartilage, the cartilage, an interface between the cartilage and the bone adjacent to the cartilage, or combinations thereof.

PDGF solutions and biocompatible matrices suitable for use in compositions, according to embodiments of methods of the present invention, are consistent with those provided hereinabove.

### Kits of the Invention

In one aspect, the present invention provides a kit comprising a first container comprising a PDGF solution and a second container comprising a biphasic biocompatible matrix. In some embodiments, the solution comprises a predetermined concentration of PDGF. The concentration of PDGF, in some embodiments, can be predetermined according to the nature of the injured or defective cartilages or bones to be treated. In some embodiments, the biphasic biocompatible matrix comprises a predetermined amount according to the type of cartilage and bone being treated. In some embodiments, the biphasic biocompatible matrix comprises a scaffolding matrix, wherein the scaffolding matrix comprises an osseous phase and a cartilage phase. In some embodiments, a syringe can facilitate dispersion of the PDGF solution in the biphasic biocompatible matrix for application at a surgical site, such as at least one site of an osteochondral defect.

The kit may also contain instructions for use for treating an osteochondral defect in a cartilage and a bone. In some embodiments, the present invention provides a kit comprising a first container comprising a PDGF solution and a second container comprising a biphasic biocompatible matrix, and instructions for mixing the PDGF solution and the biphasic biocompatible matrix for treating an osteochondral defect in a cartilage and a bone.

In another aspect, the present invention provides a kit comprising lyophilized or freeze-dried PDGF and a biphasic biocompatible matrix and instructions for hydrating the lyophilized or freeze-dried PDGF and biphasic biocompatible matrix with normal saline or other solution (e.g., water) to at least one site of an osteochondral defect and for using the resulting mixture to treat an osteochondral defect in a cartilage and/or a bone. The lyophilized or freeze-dried PDGF may be provided separately from the biocompatible matrix. For example, PDGF can be rehydrated with various solutions, including sodium acetate buffer) or it can be contained within the biphasic biocompatible matrix (e.g., by incorporating PDGF solution into the biphasic biocompatible matrix, following by lyophilization and freeze-drying.)

The following examples are provided for illustrative purposes only and are not intended to limit the scope of the invention in any manner.

### EXAMPLES

### Example 1

Evaluation of the Physical Characteristics of a Biphasic Plug for Application in Treatment of Osteochondral Defects

This study evaluated the surface topography, composition, and visualized porosity of a biphasic plug material from Orthomimetic's Chondromimetic using scanning electron microscopy.

In preparation, the plug (8.5 mm×8 mm) was placed in liquid nitrogen and vertically sectioned in two. The plug was placed in LN₂ to maintain the structural integrity of the plug.

Once halved, the plug was then mounted with double sided adhesive tape to a 26 mm round sample mounting stub. The stub was then placed into the sputter coating apparatus. The sputter coating process bombards the sample to ensure thorough coating with gold particles to increase the electrical conductivity of the sample. Once the sputter coating process was completed, the sample was then ‘grounded’ with graphite glue to discourage charging when viewed in the electron microscope.

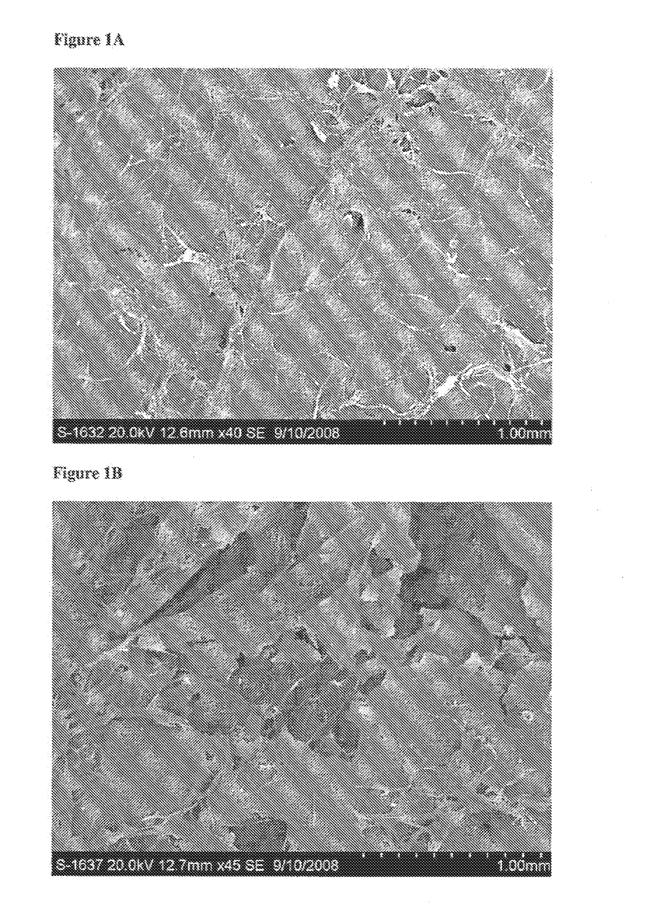

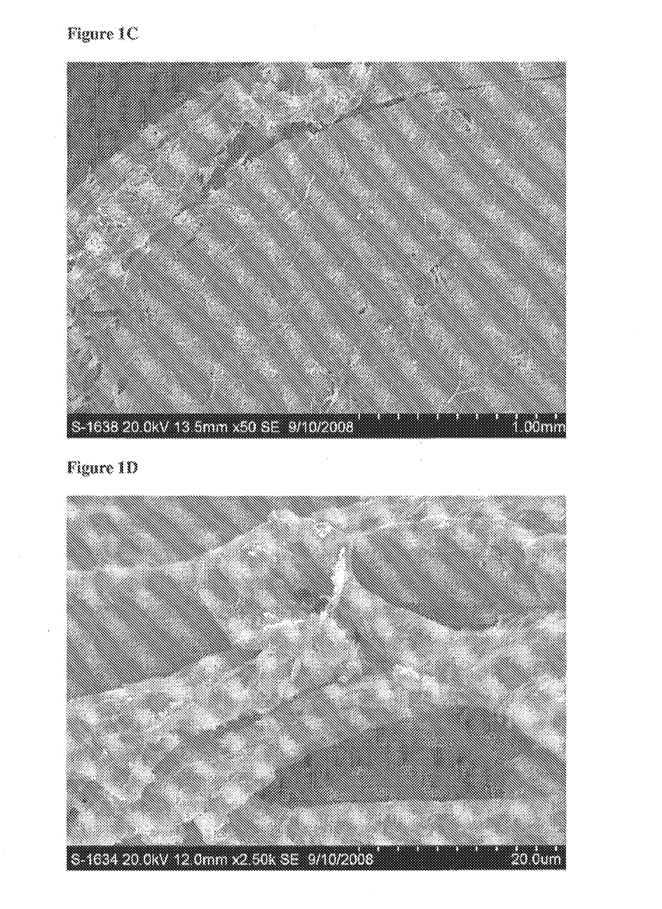

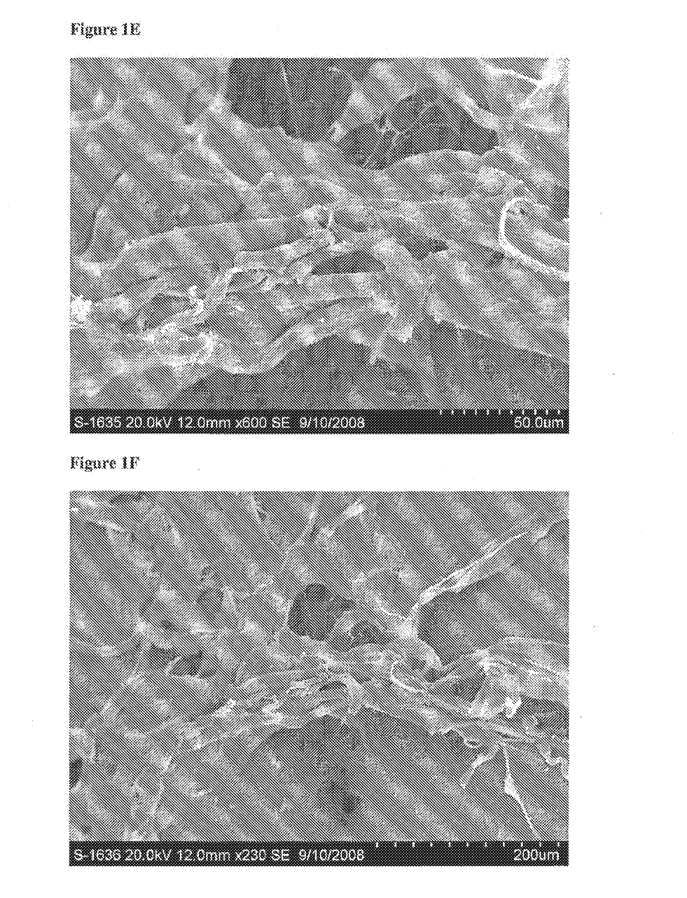

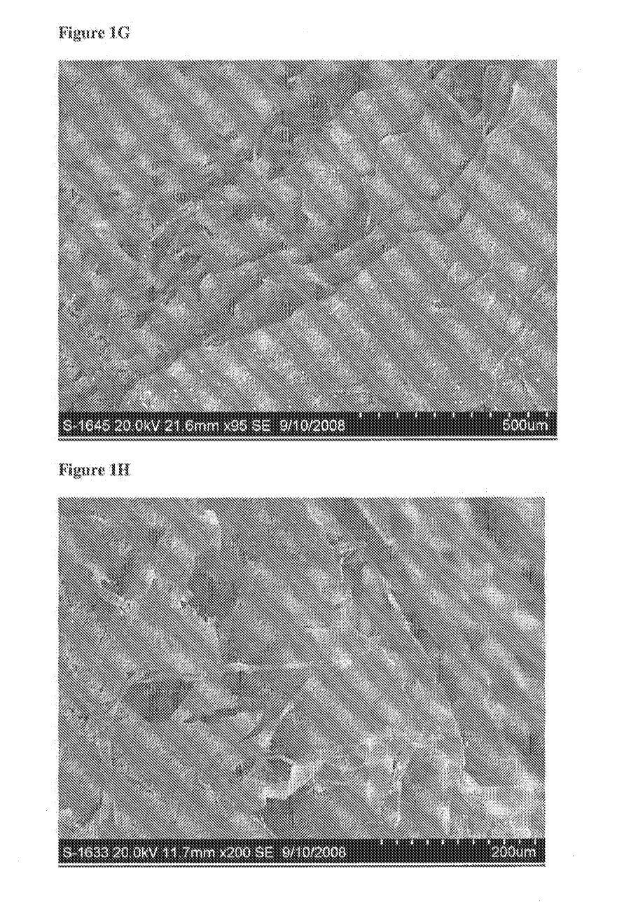

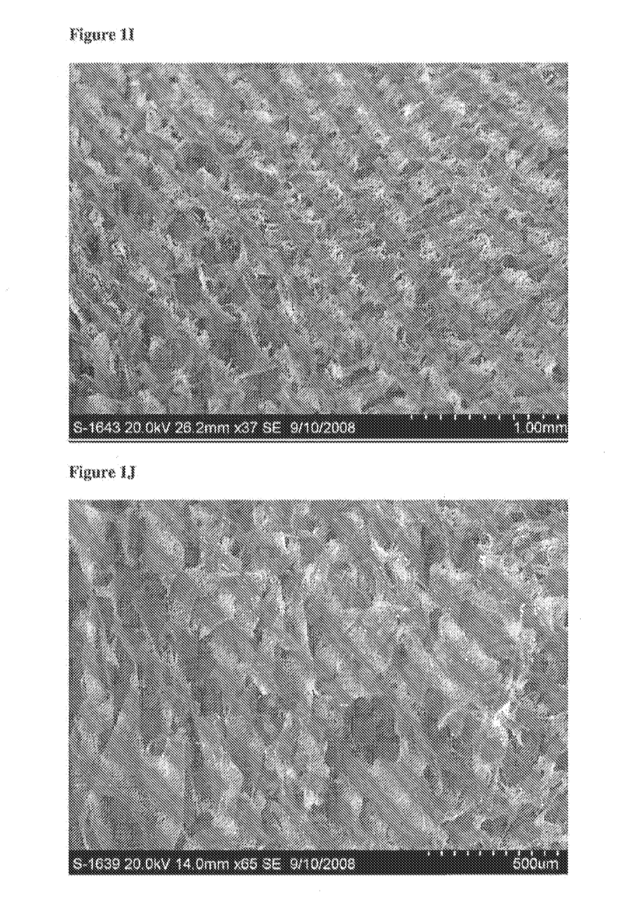

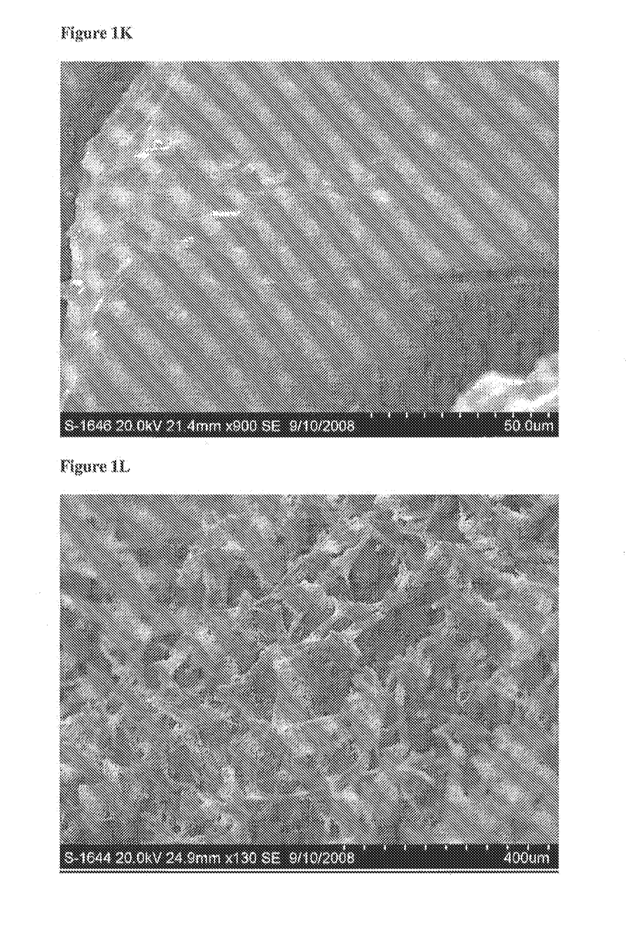

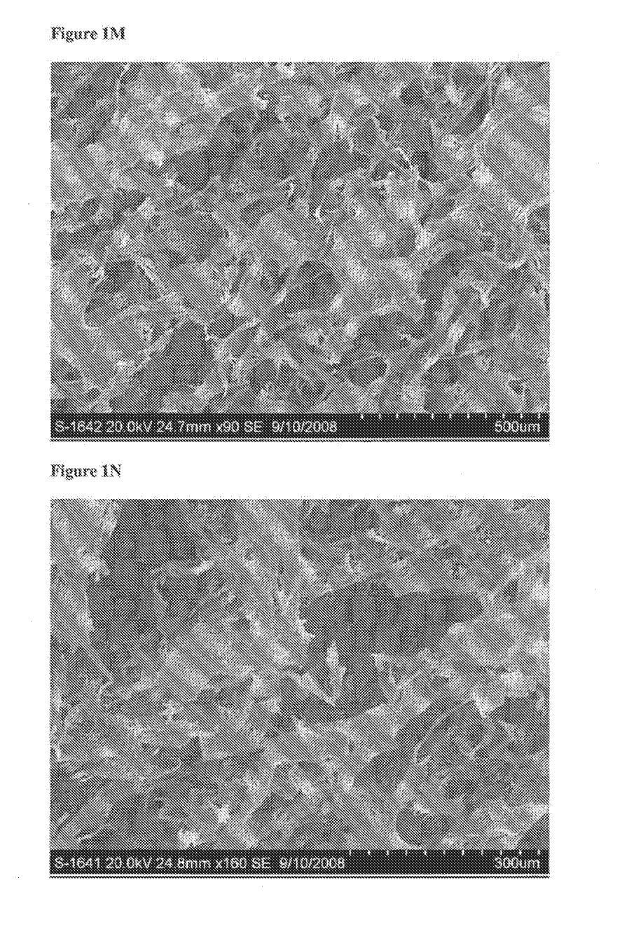

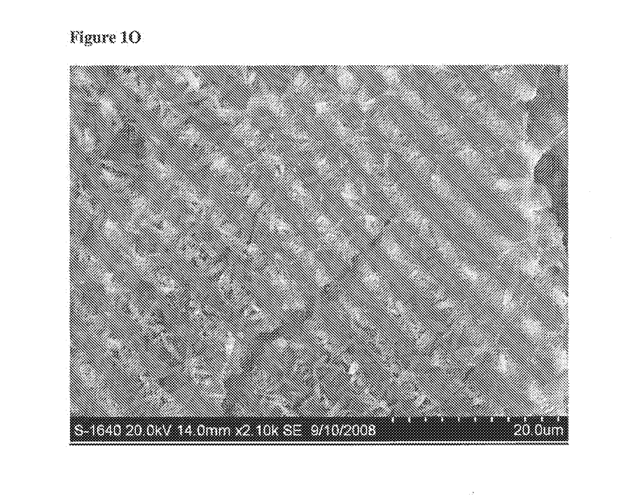

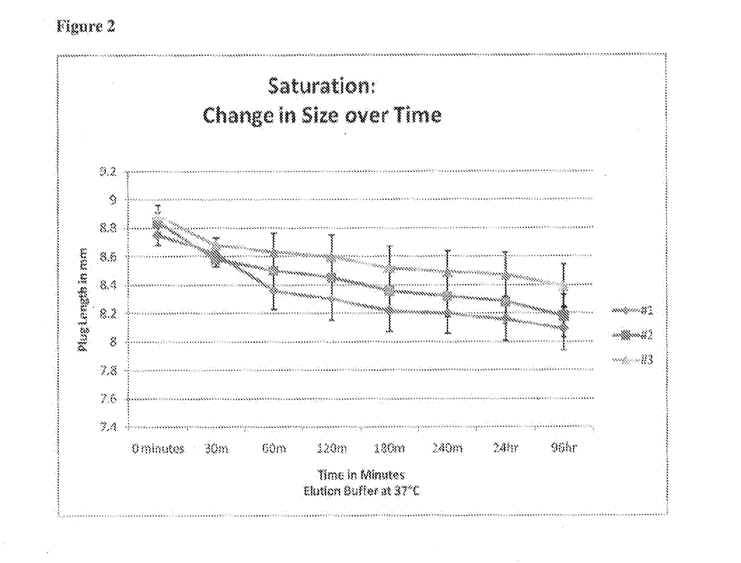

The samples were then transferred to the scanning electron microscope, and the images were recorded (FIGS. 1A-1Q).

### Example 2

### Handling Characteristics of a Biphasic Plug

This study evaluated the handling characteristics of a biphasic plug (Orthomimetic's Chondromimetic Plug), both to evaluate the progress of hydration of the plug material in a buffer solution and to determine the effects of prolonged saturation of the plug material with elution buffer over time. Methylene Blue dye was used as a visual aid to document the hydration of the plug material.

For both the hydration and saturation study components, initial observations were noted, including: size (upper and lower phase), weight, texture, rigidity, and photographically.

For the hydration component of the study, a P200 pipette was used to add Methylene Blue dyed sodium acetate buffer to the plug material in increments of 50 μL. Aqueous Methylene Blue solution was made in 20 μl Methylene Blue and 5 mL sodium acetate buffer to make 1% x/v (volume/volume) Methylene Blue. Sodium acetate buffer (20 mM sodium acetate, pH 5.99) was made with 5.44 g sodium acetate (Sigma 13505PL) and 1.8 L MQ ddH2O. The pH was adjusted to 6.0 with 200 μL 17.4 M acetic acid (Sigma 06911 ME), then q. s. to 2 L. The sodium acetate buffer was then sterilely filtered with 0.22 μm filter.

Once the plug reached visual saturation, the plug remained fully saturated for ten minutes, and then was vertically cut with a scalpel to ensure complete hydration throughout the plug material. Observations and photographs were taken. Once required volume was established for hydration, the hydration steps were repeated utilizing a syringe and needle, and additionally via syringe vacuum.

For the saturation aspect of the study, the plug was placed into a 24 well plate, fully immersed in 2.5 mL elution buffer, and placed into the 37° C. CO₂ incubator. The plate was removed at the following intervals for observation: 30 minutes; 60 minutes; 120 minutes; 180 minutes; 240 minutes; 24 hours; 96 hours. All handling was performed in a sterile test environment.

For the hydration component of the study, one plug was hydrated via calibrated pipette, one plug hydrated via syringe and needle, and one hydrated with syringe vacuum.

For the saturation component, the process was performed in triplicate.

TABLE 1
Initial Measurement of Plug Materials for Absorption Study

| Absorption Study | Upper | Lower | Width | Width | |
|---|---|---|---|---|---|
| measurements | Phase | Phase | (top) | (center) | Weight |
| #1 | 1.95 | 6.85 | 7.92 | 7.93 | 0.03116 |
| #2 | 1.87 | 7.28 | 8.26 | 8.19 | 0.02879 |
| #3 | 2.02 | 7.3 | 8.4 | 8.36 | 0.02808 |
| Average (mm) | 1.9467 | 7.1433 | 8.1933 | 8.1600 | 0.0293 |
| Standard Deviation | 0.0751 | 0.2542 | 0.2468 | 0.2166 | 0.0016 |
| % CV | 3.8556 | 3.5590 | 3.0128 | 2.6540 | 5.4964 |

TABLE 2
Initial Measurement of Plug Materials for Saturation Study

| Saturation Study | Upper | Lower | Width | Width | |
|---|---|---|---|---|---|
| Measurements | Phase | Phase | (top) | (center) | Weight |
| #1 | 1.6 | 7.15 | 8.78 | 8.4 | 0.02538 |
| #2 | 1.62 | 7.22 | 8.32 | 8.25 | 0.02822 |
| #3 | 1.67 | 7.22 | 8.39 | 8.29 | 0.02839 |
| Average (mm) | 1.6300 | 7.1967 | 8.4967 | 8.3133 | 0.0273 |
| Standard Deviation | 0.0361 | 0.0404 | 0.2479 | 0.0777 | 0.0017 |
| % CV | 2.2120 | 0.5616 | 2.9171 | 0.9343 | 6.1869 |

TABLE 3
Saturation Study: Time Point Measurements of Plug Materials in Elution Buffer at 37° C.

| Saturation | 0 | 30 | 60 | 120 | 180 | 240 | 24 | 96 | Total |
|---|---|---|---|---|---|---|---|---|---|
| Study | minutes | minutes | minutes | minutes | minutes | minutes | hours | hours | Loss |
| #1 | 8.75 | 8.62 | 8.36 | 8.3 | 8.22 | 8.2 | 8.16 | 8.09 | 0.66 |
| #2 | 8.84 | 8.58 | 8.5 | 8.45 | 8.36 | 8.32 | 8.28 | 8.18 | 0.66 |
| #3 | 8.89 | 8.68 | 8.63 | 8.6 | 8.52 | 8.49 | 8.47 | 8.39 | 0.5 |
| Average | 8.8267 | 8.6267 | 8.4967 | 8.4500 | 8.3667 | 8.3367 | 8.3033 | 8.2200 | 0.6067 |
| (mm) | | | | | | | | | |
| Standard | 0.0709 | 0.0503 | 0.1350 | 0.1500 | 0.1501 | 0.1457 | 0.1563 | 0.1539 | 0.0924 |
| Deviation | | | | | | | | | |
| % CV | 0.8038 | 0.5834 | 1.5892 | 1.7751 | 1.7942 | 1.7479 | 1.8825 | 1.8728 | 15.2268 |

In the absorption aspect of the study, it was found that complete absorption of the plug material was achieved with 450 μL loading buffer. This volume ensured complete saturation throughout plug material. Loading the plug material via vacuum syringe proved more difficult to properly hydrate the plug, and required more manipulation of the plug within the syringe to sufficiently hydrate with the buffer. Once fully hydrated, the plug material retained the buffer solution, and acted very much as a sponge in texture and rigidity.

For the saturation component of the study, it was determined no significant shrinkage or change to the physical aspect of the plug material was identified after immersion in buffer solution, placing at 37° C., for up to 96 hrs of incubation. The top phase of the plug did exhibit greater buoyancy compared to the bottom phase, as evident by the reorientation of the plug from lying on its side to top side up once placed in elution buffer. The plug material exhibited excellent shape memory at all time points. No particulate matter or cloudiness of elution buffer was noted throughout the experiment.

### Example 3

### Evaluation of the Release of rhPDGF-BB (Recombinant Human Platelet-Derived Growth Factor-BB) from a Biphasic Plug Material

This study evaluated the kinetics of rhPDGF-BB release from a biphasic matrix plug (Chondromimetic (Orthomimetics)) over time.

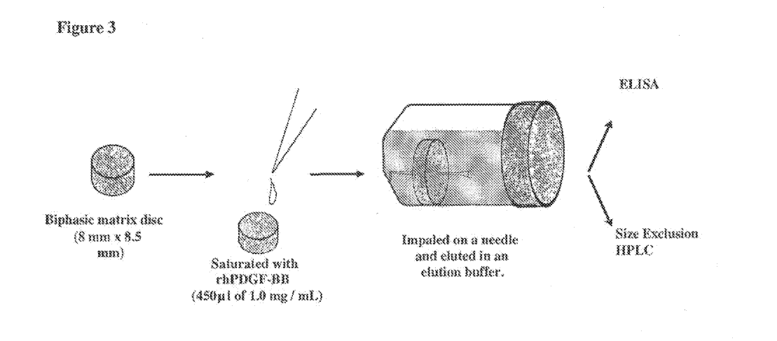

In sterile conditions, each plug was stabilized over a Sarstedt 15 ml conical polypropylene tube with a 27G1/2″ needle and syringe (plunger removed from syringe). Each of Orthomimetic's Chondromimetic matrix plug (×3; 8.5 mm×8 mm) was loaded with 450 μL rhPDGF-BB (e.g., 0.3 mg/ml or 1.0 mg/ml), and then allowed to sit at room temperature saturated within conical tube for ten minutes (see FIG. 3).

Following incubation, 9 ml elution buffer (1% L-glutamine, 1% Pen-Strep, 2% FBS (heat inactivated, Gamma-irradiated), 2.5% HEPES) was placed into 15 ml conical tubes, numbered 1-6. Conical tubes numbered 1-3 contained loaded sample plugs, tubes numbered 4-6 served as controls, where 450 μL rhPDGF-BB was added directly to the elution buffer (1% L-glutamine, 1% Pen-Strep, 2% FBS (heat inactivated, Gamma-irradiated), and 2.5% HEPES). See Table 4. The conical tubes were then placed onto a rocking platform located within a 37° C. Incubator.

At each time point: 10 minutes, 1 hour, 8 hours, 24 hours, the conical tubes were removed from the rocking platform, and returned to the sterile laminar flow hood for complete wash out collection of all elution buffer for use in an ELISA (Enzyme-Linked ImmunoSorbent Assay).

Once all the wash out had been collected into sterile conical tubes, 9 mls of fresh elution were placed into each sample conical and returned to the rocking platform. The collected wash out samples were placed into the 2-4° C. cold box.

After all samples had been collected, an ELISA was performed to determine rhPDGF-BB concentration within the wash out at each time point.

TABLE 4
Sample Preparation

| | | | | | | |
|---|---|---|---|---|---|---|
| | | Initial | | | | |
| | | Incubation | | | | |
| Sample | | Time | | | | |
| Number | Test Material | (Minutes) | +10 min | +1 hr | +8 hr | +24 hr |

| | | | |
|---|---|---|---|
| 1 | Orthomimetic Matrix Plug | 10-12 | Sample* |
| | loaded with 450 μL | minutes | |
| | rhPDGF-BB | | |
| 2 | Orthomimetic Matrix Plug | 10-12 | Sample* |
| | loaded with 450 μL | minutes | |
| | rhPDGF-BB | | |
| 3 | Orthomimetic Matrix Plug | 10-12 | Sample* |
| | loaded with 450 μL | minutes | |
| | rhPDGF-BB | | |
| 4 | rhPDGF-BB Control | 10-12 | Sample* |
| | | minutes | |
| 5 | rhPDGF-BB Control | 10-12 | Sample* |
| | | minutes | |
| 6 | rhPDGF-BB Control | 10-12 | Sample* |
| | | minutes | |

*Sampling involved removing elution buffer from the matrices and then replacing it with the same volume of fresh elution buffer.

### A. ELISA Assay Procedure

Diluted capture reagent in 100λ was added to each well of a 96-well plate (Corning 3590). Adhesive plate cover was covered, and the diluted capture reagent was allowed to coat at room temperature overnight on an orbital shaker.

Each well was then aspirated and washed with 300 μl PBST (1× phosphate buffered saline with Tween 20).

Sample diluent (elution buffer) in 200 μl was added to block for at least 2 hours at room temperature on plate rocker.

Each well was then aspirated and washed with 300 μl PBST for 3 times.

A standard curve of rhPDGF-BB was prepared using the lot of rhPDGF-BB used in the test samples. The rhPDGF-BB was then diluted to 10 ng/ml using the elution buffer as a diluent. Serial doubling dilutions were made to 0.15625 ng/ml.

Samples 1-7 were diluted for assay using the elution buffer as diluent (see Table 5).

TABLE 5
Dilution Series Chart

| Tube # | | 10 min | 1 h | 8 h | 24 h |
|---|---|---|---|---|---|
| 1-3 | D-1 | 1000 | 200 | 100 | 50 |
| | D-2 | 5000 | 1000 | 200 | 100 |
| 4-6 | D-1 | 2000 | 100 | 20 | No |
| | | | | | dilution |
| | D-2 | 10000 | 500 | 100 | 5 |

Each sample and standard in 100 μl were pipetted in duplicate into plate, following assay template. Plates were covered with adhesive film and incubated 1 to 2 hours at room temperature while rocking.

Each well was then aspirated and washed with 300 μl PBST for 3 times.

Detection antibody in 100 μl was added to each well, covered with adhesive film and rocked for 1 to 1½ hours.

Each well was then aspirated and washed with 300 μl PBST for 3 times.

Streptavidin-HRP was diluted in 1:200 using reagent diluent, added 100 μl to each well, covered with aluminum foil, and incubated for 20 minutes at room temperature.

Each well was then aspirated and washed with 300 μl PBST for 3 times.

Sure Blue in 100 μl from KPL (KPL Protein Research Products, Gaithersburg, Md.) was added, covered with aluminum foil, and incubated for 20 minutes at room temperature. TMB (tetramethylbenzemidine) was protected from light at all times. Blue color appeared with time.

HCL (1N) in 50 μl was added to each well to quench the reaction. Wells containing blue color appeared yellow.

Optical density of each well was determined within 30 minutes of addition of Stop solution in a microplate reader set to 450 nm with wavelength correction of 540 nm. Optical density readings were exported to MicroSoft Excel for analysis.

TABLE 6
Concentration of rhPDGF-BB Released and Total Release of rhPDGF-BB from
the Orthomimetic Plug Matrix at time points through 24 hours.

| | Concentration of | | | | | | | |
|---|---|---|---|---|---|---|---|---|
| | rhPDGF-released (ng/ml) | | | | Amount of rhPDGF-released (ng) | | | |
| | 10 min | 1 h | 8 h | 24 h | 10 min | 1 h | 8 h | 24 h |
| Ortho Plug | 5948.2 | 2179.0 | 741.3 | 357.2 | 53534.0 | 19611.4 | 6671.6 | 3214.5 |
| Ortho Plug | 7158.9 | 2010.0 | 501.9 | 246.7 | 64430.5 | 18089.7 | 4517.1 | 2220.5 |
| Ortho Plug | 5924.8 | 2084.7 | 650.1 | 259.7 | 53322.9 | 18762.1 | 5851.0 | 2337.6 |
| Mean | 6344.0 | 2091.2 | 631.1 | 287.9 | 57095.8 | 18821.0 | 5679.9 | 2590.9 |
| STDEV | 705.9 | 84.7 | 120.8 | 60.4 | 6352.9 | 762.6 | 1087.4 | 543.2 |
| CV | 11.1 | 4.1 | 19.1 | 21.0 | 11.1 | 4.1 | 19.1 | 21.0 |
| Control | 12373.1 | 248.3 | 15.6 | 1.9 | 111357.7 | 2234.6 | 140.0 | 17.1 |
| Control | 12243.1 | 290.8 | 15.4 | 1.8 | 110187.9 | 2617.6 | 139.0 | 16.2 |
| Control | 12711.5 | 283.3 | 17.9 | 1.9 | 114403.6 | 2549.3 | 161.5 | 17.2 |
| Mean | 12442.6 | 274.1 | 16.3 | 1.9 | 111983.0 | 2467.2 | 146.8 | 16.9 |
| STDEV | 241.8 | 22.7 | 1.4 | 0.1 | 2176.3 | 204.3 | 12.7 | 0.5 |
| CV | 1.9 | 8.3 | 8.7 | 3.1 | 1.9 | 8.3 | 8.7 | 3.1 |

TABLE 7
Cumulative rhPDGF-BB Released and % Release as Compared to Control.

| | Cumulative amount of | | | | % rhPDGF-released | | | |
|---|---|---|---|---|---|---|---|---|
| | rhPDGF-released (ng) | | | | of control (ng) | | | |
| | 10 min | 1 h | 8 h | 24 h | 10 min | 1 h | 8 h | 24 h |
| Ortho | 53534.0 | 73145.4 | 79817.0 | 83031.5 | 47.8 | 63.9 | 69.7 | 72.4 |
| Plug | | | | | | | | |
| Ortho | 64430.5 | 82520.2 | 87037.2 | 89257.7 | 57.5 | 72.1 | 76.0 | 77.9 |
| Plug | | | | | | | | |
| Ortho | 53322.9 | 72084.9 | 77936.0 | 80273.6 | 47.6 | 63.0 | 68.0 | 70.0 |
| Plug | | | | | | | | |
| Mean | 57095.8 | 75916.8 | 81596.7 | 84187.6 | 51.0 | 66.3 | 71.2 | 73.5 |
| STDEV | 6352.9 | 5743.2 | 4804.6 | 4602.3 | 5.7 | 5.0 | 4.2 | 4.0 |
| CV | 11.1 | 7.6 | 5.9 | 5.5 | 11.1 | 7.6 | 5.9 | 5.5 |
| Control | 111357.7 | 113592.3 | 113732.2 | 113749.3 | 101.0 | 103.0 | 103.2 | 103.2 |
| Control | 110187.9 | 112805.5 | 112944.5 | 112960.8 | 99.9 | 102.3 | 102.4 | 102.5 |
| Control | 114403.6 | 116952.9 | 117114.4 | 117131.6 | 103.8 | 106.1 | 106.2 | 106.2 |
| Mean | 111983.0 | 114450.2 | 114597.0 | 114613.9 | 101.6 | 103.8 | 103.9 | 104.0 |
| STDEV | 2176.3 | 2202.8 | 2215.4 | 2215.7 | 2.0 | 2.0 | 2.0 | 2.0 |
| CV | 1.9 | 1.9 | 1.9 | 1.9 | 1.9 | 1.9 | 1.9 | 1.9 |

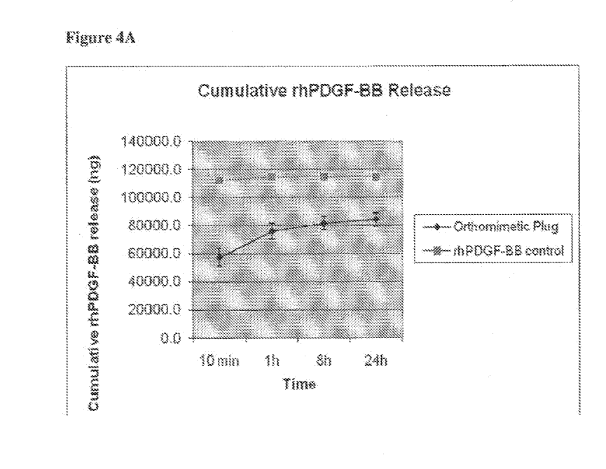

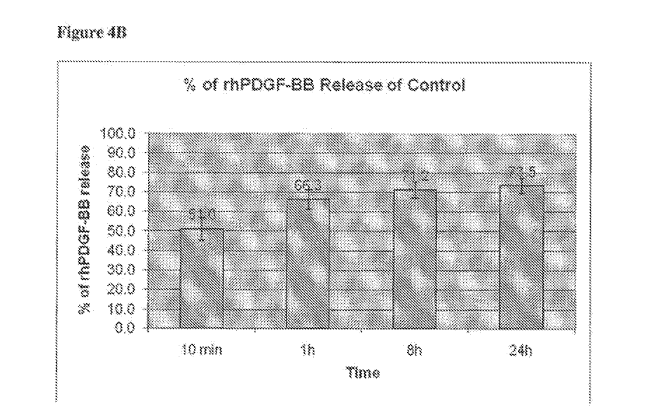

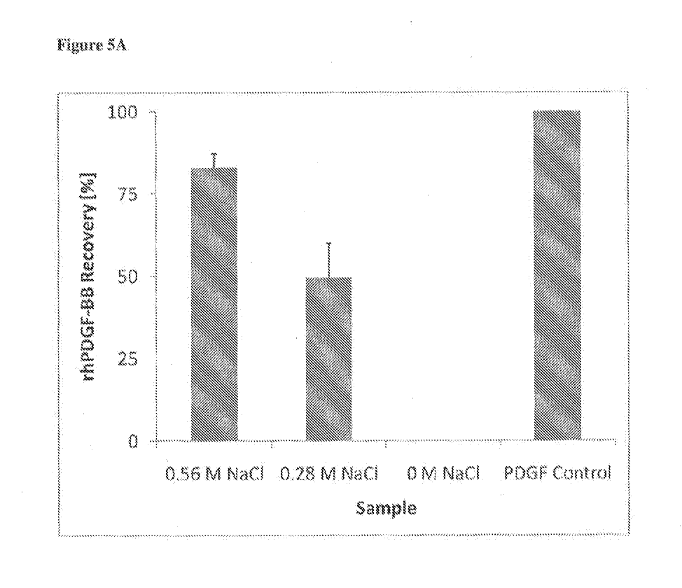

There was an initial bolus release of 51% of rhPDGF-BB after ten minutes, followed by a slower phase of release over the remaining 23 hour study period. The cumulative release of rhPDGF-BB was 70-75% (mean value 73.5%) as compared to control from the matrix material after 24 hours (see Tables 6-7 and FIGS. 4A-4B). Complete recovery of rhPDGF-BB was not achieved utilizing this in vitro experiment design. A portion of the added protein may become covalently cross linked to the material and is not released over the time frame measured in this study. Positive controls run as part of the study design indicated that the assays were functioning normally for the detection of rhPDGF-BB. Combination of rhPDGF-BB with biphasic matrix did not negatively impact PDGF-BB biochemical stability. The PDGF-receptor binding efficiency, as determined by non-liner regression, of the released rhPDGF-BB was equivalent to that observed for the control rhPDGF-BB.

### Example 4

### Effect of a Biphasic (RIVERSIDE®) Plug from Orthomimetics on Properties of Recombinant Human Platelet Derived Growth Factor BB (rhPDGF-BB)

This study was to evaluate the structural and functional integrity of rhPDGF-BB after combination with RIVERSIDE® plug from Orthomimetics via size exclusion and reversed phase HPLC and binding affinity to rhPDGF-BB receptor determined by ELISA.

### A. Test Methods

Two RIVERSIDE® plugs were cut in quarters. Plugs were soaked with 100 μl of rhPDGF-BB (e.g., 1.0 mg/mL in 20 mM sodium acetate buffer) to each quarter of plug labeled with samples 1-6 or with the same volume of the sodium acetate (NaAc) buffer (controls 7, 8) and incubated for 10 minutes at room temperature.

Microcentrifuge tubes were filled with 300 μl of elution buffer containing different salt concentration based as shown in Table 8.

TABLE 8
Sample Preparation

| | | | Final concentration |
|---|---|---|---|
| | Saturation | | of NaCl (mM) in |
| Sample No. | reagent | Elution buffer (20 mM) | elution Buffer |
| 1 | rhPDGF | NaAC | 0.56 |
| 2 | rhPDGF | NaAC | 0.56 |
| 3 | rhPDGF | NaAC | 0.28 |
| 4 | rhPDGF | NaAC | 0.28 |
| 5 | rhPDGF | NaAC | 0 |
| 6 | rhPDGF | NaAC | 0 |
| 7 | NaAC | NaAC | 0.56 |
| 8 | NaAC | NaAC | 0 |

Tubes were placed on a rocker in a 37° C. incubator and rocked for 1 hour.

After 1 hour, tubes were removed from the incubator and the elution buffer was transferred from each tube to a microtube.

Samples were centrifuged at 14,000 rpm at 20° C. for 5 minutes. Then 150 μl of each sample was transferred into a glass vial for size exclusion HPLC, 90 μl to a micro tube for reversed phase HPLC, and 10 μl for PDGF detection assay using DuoSet ELISA.

### Size Exclusion HPLC

Sample at 100 μl was loaded on the size exclusion column from an auto sampler using automatic injector and eluted from the column equilibrated with 0.4 M NaCl in 0.05 M sodium acetate, pH 4.0 at flow rate 0.8 ml/min at room temperature. Samples were kept at 4° C. for the time of chromatography.

### Reversed Phase HPLC

Samples, 90 μl each, were first denatured with 200 mM dithiothreitol and 4 M guanidine hydrochloride for 5 min at 50° C. and then loaded on a C₁₈ reversed phase column eluted with a gradient 24-45% acetonitrile in 0.06% trifluoroacetic acid at 1.2 ml/min following the Test Procedure QCT004. Absorbance at 214 nm was used for data collection.

### ELISA Binding Assay

Samples at 4 folds dilution were diluted to 10 ng/ml using Elution buffer and serial 2-fold dilutions were prepared for total 7 dilutions in duplicates. Then PDGF detection assay using DuoSet assay was conducted following standard protocol from R & D Lab for this assay. No deviations were recorded for this study.

### B. Results

Profiles of rhPDGF-BB from SEC (Size Exclusion Chromatography) reflected changes in its native structure and/or presence of soluble components eluted from the plugs. There was some low molecular weight background eluted from the plug material at all salt concentrations. However, rhPDGF-BB was released from the material only if salt was present in the elution buffer indicating that rhPDGF-BB adheres to the plug by ionic interactions. At lower salt concentrations (e.g., 0.28 M NaCl), a small high molecular weight peak appeared at elution time ˜8 minutes (not visible in the blank elution), indicating perhaps that some rhPDGF-BB aggregation occurred. This was not visible at higher salt concentrations (e.g., 0.56 M NaCl).

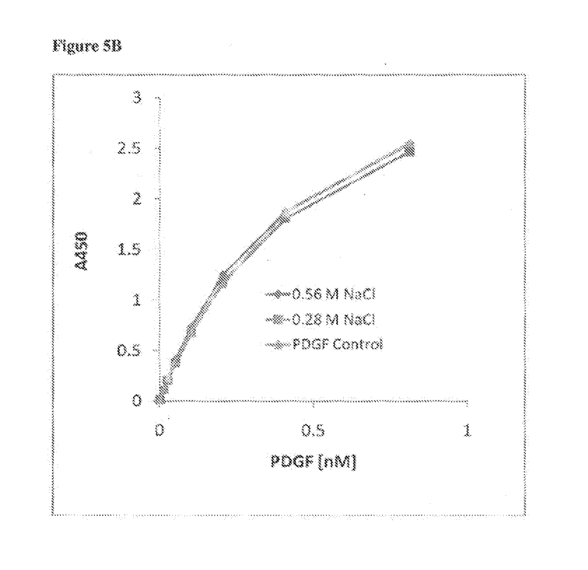

Integration of the rhPDGF-BB peaked in the SEC profiles provided the base for quantitation of the amounts of the growth factor eluted from the biphasic biocompatible matrix. Standard curve performed from a set of six standards of different concentrations of rhPDGF-BB ran before and after the sample set was used for calibration of the assay (R²=1.0) ran on SEC. The data shown in FIG. 5A show that the recovery of PDGF was dependent on the concentration of sodium chloride in the elution buffer.

Reversed phase HPLC (RPHPLC) profile of rhPDGF-BB eluted from the plugs showed that no changes/modifications in the denatured structure of the growth factor occurred due to its interaction with the biphasic biocompatible matrix. It was also confirmed that no elution of rhPDGF-BB from the biphasic biocompatible matrix in the absence of salt in the elution buffer.

Efficiency of rhPDGF-BB binding to its receptor was evaluated by ELISA assay with receptor coated to the plate. FIG. 5B shows that no significant changes were visible in the binding curves obtained at 8 different concentrations of rhPDGF-BB. Related dissociation constants were shown in Table 9.

TABLE 9
Dissociation Constants of rhPDGF-BB calculated from the binding curves
in FIG. 5D by non-linear regression using SigmaStat.

| | Average | | | | | |
|---|---|---|---|---|---|---|
| | K_{D} | | SD | | | |
| Name | [nM] | Size | [nM] | % CV | P | R² |
| 0.56M NaCl | 0.4311 | 8 | 0.0125 | 2.90 | <0.0001 | 0.9998 |
| 0.28M NaCl | 0.4739 | 8 | 0.0098 | 2.07 | <0.0001 | 0.9999 |
| PDGF Control | 0.5052 | 8 | 0.0214 | 4.24 | <0.0001 | 0.9995 |

Based on the results of these experiments, elution of rhPDGF-BB from the RIVERSIDE® plug is salt dependent. rhPDGF-BB can form aggregates after elution at lower salt concentrations. Further, no changes in the denatured structure of rhPDGF-BB were observed (oxidation, cleavage, or other chemical modification). Finally, rhPDGF-BB is mostly unaffected by its interaction with the biphasic biocompatible matrix.

### Example 5

### Osteochondral Defects Treatment Studies Using Biphasic Biocompatible Matrix and rhPDGF-BB

### A. Study I

The study includes 3 groups of Boer-cross male castrated goat. The first group (group 1) consists of six to eight goats that receive biphasic biocompatible matrix plug (Chondromimetic (Orthomimetics)) alone. The second (Group 2) and the third groups (Group 3) also consist of six to eight goats and receive one of two concentrations of rhPDGF-BB (0.3 mg/ml or 1.0 mg/ml), disposed within a biphasic biocompatible matrix plug. On the initial day of the study, all animals undergo bilateral creation of a grade 3 defect within the medial femoral condyle (8-10 mm in diameter and 6-8 mm deep). For Groups 2 and 3, a biphasic biocompatible matrix plug is implanted within the defect in one condyle. The defect is a hole running down through the condyle into the bone adjacent to the condyle (or its underlying bone), so the hole includes the hole in the condyle, the bone adjacent to the condyle, and also the interface between the condyle and the bone adjacent to the condyle. The contralateral defect in the same animal is treated with biphasic biocompatible matrix plug and sodium acetate buffer. Alternatively, a biphasic biocompatible matrix plug is implanted within the defect in both condyles in the same animal in groups 2 and 3. The goats are maintained for a period of 2 weeks, 12 weeks, or 26 weeks, at which time, they are euthanized, and the implanted area is prepared for histological examination, MRI (Magnetic Resonance Imaging), gross evaluation, photodocumentation, synovial fluid analysis (time zero and at sac), mechanical stiffness testing (all can be done on the same speciemen).

Defects in the articular cartilage of the medial femoral condyle and the bone adjacent to the condyle treated with compositions comprising the PDGF solution disposed in the biphasic biocompatible matrix demonstrate enhanced healing and repair including the formation and growth of the medial femoral condyle and its underlying bone.

### B. Study II

The study includes 4 groups of Boer-cross male castrated goat. The first group (Group 1) consists of four goats without articular cartilage grade 3 defect and receives no treatment. The second group (Group 2) consists of eight goats with articular cartilage grade 3 defect and receive biphasic biocompatible matrix plug (Chondromimetic (Orthomimetics)) alone. The third (Group 3) and the fourth groups (Group 4) also consist of eight goats and receive one of two concentrations of rhPDGF-BB (0.3 mg/ml or 1.0 mg/ml), disposed within a biphasic biocompatible matrix plug. On the initial day of the study, animals in Groups 2-4 undergo bilateral creation of a defect within the medial femoral condyle (8-10 mm in diameter) and proximal trochlear sulcus. For Groups 2 to 4, a biphasic biocompatible matrix plug is implanted within the defect in one condyle and trochlear. The defect is a hole running down through the cartilage into the bone so the plug is placed into the defect. The hole includes the hole in the medial femoral condyle or in the proximal trochlear sulcus, the bone adjacent to the medial femoral condyle or the proximal trochlear sulcus, and also the interface between the medial femoral condyle or the proximal trochlear sulcus and the bone adjacent to the medial femoral condyle or the proximal trochlear sulcus. The contralateral defect in the same animal is treated with biphasic biocompatible matrix plug and sodium acetate buffer. Alternatively, a biphasic biocompatible matrix plug is implanted within the defect in both condyles or in both the proximal trochlear sulci in the same animal in Groups 2 to 4. The goats are maintained for a period of 25 weeks and 52 weeks, at which time, they are euthanized, and the implanted area is prepared for histological examination, MRI (Magnetic Resonance Imaging), gross evaluation, photodocumentation, synovial fluid analysis (time zero and at sac), mechanical stiffness testing (all can be done on the same speciemen).

Defects in the articular cartilage of the medial femoral condyle or of the proximal trochlear sulcus and the bone adjacent to the medial femoral condyle or the proximal trochlear sulcus treated with compositions comprising the PDGF solution disposed in the biphasic biocompatible matrix demonstrate enhanced healing and repair including the formation and growth of medial femoral condyle or proximal trochlear sulcus and its underlying bone.

### Example 6

### In Vitro Cytocompatibility Study: Cell Seeding onto Biphasic Biocompatible Matrix Disc

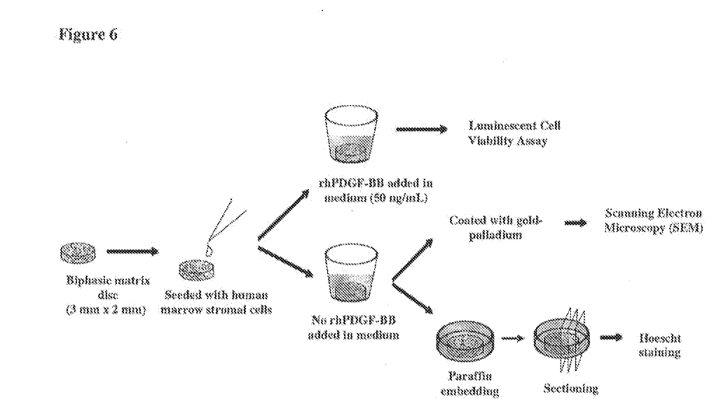

Biphasic matrix discs were seeded with human marrow stromal (hMSC) cells with 5×10⁴ hMSC cells in 20 μl complete growth media without rhPDGF-BB or biphasic matrix discs seeded with 1×10⁴ hMSC cells in 20 μl of starvation media (0.3% FBS) with or without rhPDGF-BB (50 ng/mL). Biphasic matrix discs were incubated at 37° C. in 5% CO₂ incubator for 48 hours prior to removal for the luminescent cell viability assay, histology, and scanning electron microscopy (SEM) (FIG. 6).

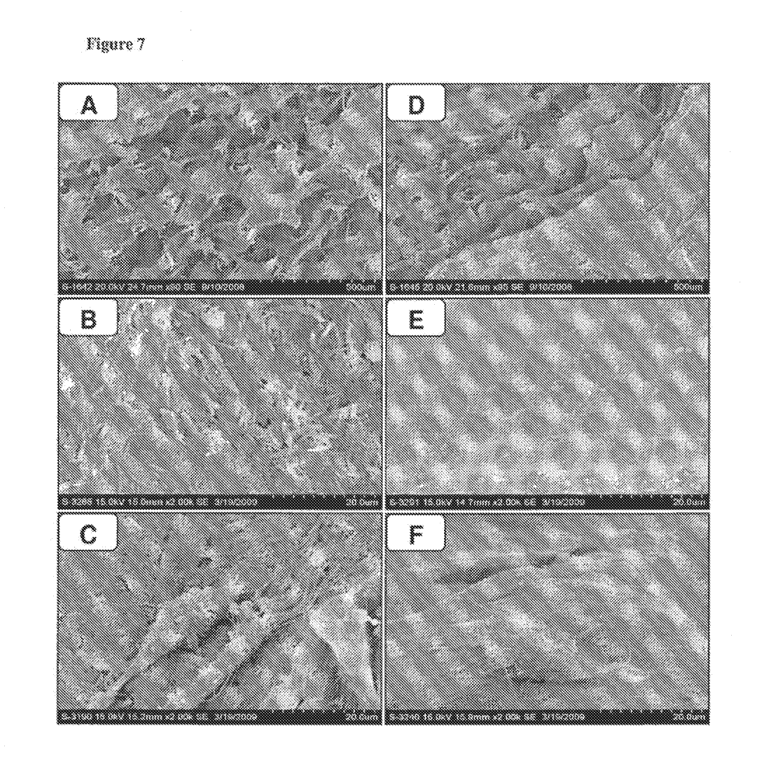

The SEM images of the biphasic matrix disc showed the dual-layer structure of the scaffolding material (FIG. 7A-7F). The lower phase of the biphasic matrix disc was comprised of cross-linked fibers with a calcium phosphate coating without cells (FIGS. 7A and 7B) or with hMSCs cells (FIG. 7C). The top layer parallel fiber alignment was shown without cells (FIGS. 7D and 7E) or with hMCs cells (FIG. 7F). Histology data confirmed the presence of cells distributed throughout the matrix.

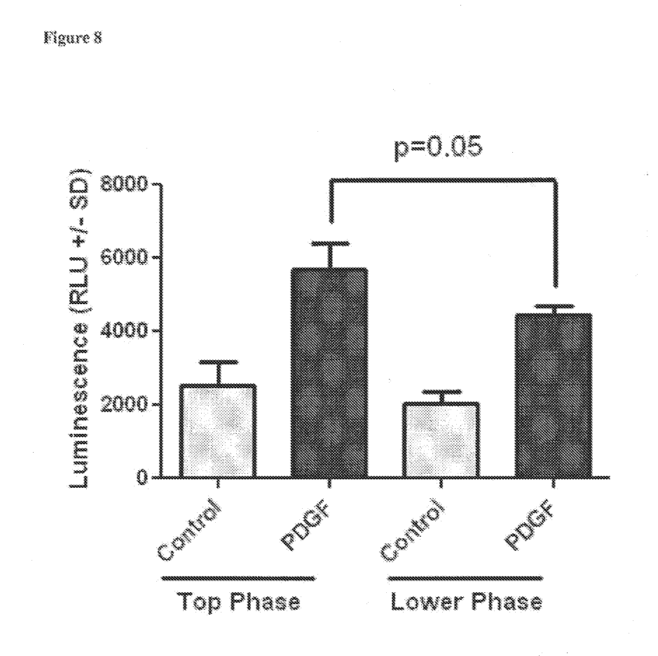

For luminescent cell viability ATP assay, the hMSC seeded biphasic matrix discs were added alone or added with rhPDGF-BB to the cell suspension at 2-day time point. The hMSC cells were observed to readily attach to both top and lower phases on the biphasic matrix disc. The luminescent signal was proportional to the amount of ATP present, which was directly proportional to number of live cells present. The assay showed that there was statistical significance (P<0.05) between the rhPDGF-BB treated and control groups for both the top and lower phases (FIG. 8). Cell number increased significantly at two days for rhPDGF-BB treated cells compared to cells in media alone in both top and lower phases of the biphasic matrix disc.

### Example 7

### Evaluation of rhPDGF-BB Combined with a Bi-Phasic Biocompatible Matrix for Osteochondral Defect Repair in a Caprine Model

The goal of this study was to determine the impact of augmentation of osteochondral defect repair using a bi-phasic biocompatible matrix plug/implant (e.g., Chondromimetic (Orthomimetics)) combined with rhPDGF-BB.

### Materials and Methods

The following materials were used: 1) 1.0 mg/ml rhPDGF-BB in 20 mM sodium acetate buffer, pH 6.0 (Lot/Batch #: QCPDGF-090209-1.0); 2) 0.15 mg/ml (±10%) rhPDGF-BB in 20 mM sodium acetate buffer, pH 6.0 (Lot/Batch #: QCPDGF-090209-0.15); and 3) Chondromimetic Implant (Orthomimetics Ltd, Cambridge, UK), porous collagen implant, 8.5 mm diameter×8 mm depth (Lot/Batch #: CM019, Expiration: 02/2010)), and 4) 20 mM sodium acetate buffer, pH 6.0+/−0.5 (Lot/Batch #: QCAB040709).

Retained and returned samples of 0.15, and 1.0 mg/ml concentrations of rhPDGF-BB were tested for concentration by UV and stability by rpHPLC (reversed phase high performance liquid chromatography). All returned samples had concentrations and stability profiles which were comparable to the retained samples.

A total of 32 skeletally mature castrated male, Nubian Boer-cross goats were used for this study. They were acquired from an approved, USDA source (Thomas D. Morris, Inc.). All animals were between 3 to 4 years old at the start of the study. The goat was chosen because of the large stifle joint size, ease of handling and use in other cartilage repair studies. See, Shahgaldi B F et al., J Bone Joint Surg, 73(1): 57-64 (1991).

### Study Design

This study was designed as a dose-range finding and efficacy study, containing 5 surgical groups. A control group with no treatment to the osteochondral defect, a control group with the Chondromimetic type I collagen implant saturated with 20 mM sodium acetate (buffer), an experimental group with the Chondromimetic type I collagen implant saturated with 0.5 cc of 0.030 mg/ml rhPDGF-BB in buffer, an experimental group with the Chondromimetic type I collagen implant saturated with 0.5 cc of 0.15 mg/ml rhPDGF-BB in buffer, and an experimental group with the Chondromimetic type I collagen implant saturated with 0.5 cc of 1.0 mg/ml rhPDGF-BB in buffer. The allocation of the groups to the animals is described in Table 10 below.

TABLE 10
Treatment Groups

| | | Implant | | Splint | | |
|---|---|---|---|---|---|---|
| | | (Chondromimetic | Growth | Time | Survival | # |
| Group | Implant Site | type I collagen) | Factor | (days) | (weeks) | Animals |
| 1 | MFC | No | None | 14 ± 1 | 12 ± 0.5 | 4 |
| 2 | MFC | Yes | NaAc | 14 ± 1 | 12 ± 0.5 | 7 |
| | | | Buffer | | | |
| 3 | MFC | Yes | rhPDGFBB | 14 ± 1 | 12 ± 0.5 | 7 |
| | | | 15 μg | | | |
| 4 | MFC | Yes | rhPDGFBB | 14 ± 1 | 12 ± 0.5 | 7 |
| | | | 75 μg | | | |
| 5 | MFC | Yes | rhPDGFBB | 14 ± 1 | 12 ± 0.5 | 7 |
| | | | 500 μg | | | |

| | |
|---|---|
| Total | 32 |

MFC: Medial Femoral Condyle

Treatments were randomized using the random number generator in Excel, as described in Table 11 below.

TABLE 11
Treatment randomization

| Animal Number | Treatment |
|---|---|
| 1 | 1 |
| 2 | 3 |
| 3 | 2 |
| 4 | 5 |
| 5 | 4 |
| 6 | 2 |
| 7 | 3 |
| 8 | 5 |
| 9 | 4 |
| 10 | 5 |
| 11 | 1 |
| 12 | 4 |
| 13 | 3 |
| 14 | 2 |
| 15 | 3 |
| 16 | 3 |
| 17 | 4 |
| 18 | 5 |
| 19 | 1 |
| 20 | 3 |
| 21 | 4 |
| 22 | 3 |
| 23 | 4 |
| 24 | 5 |
| 25 | 4 |
| 26 | 2 |
| 27 | 5 |
| 28 | 2 |
| 29 | 1 |
| 30 | 2 |
| 31 | 2 |
| 32 | 5 |

Group Assignment

| Treatment Group | Treatment |
|---|---|
| 1 | Empty Defect |
| 2 | Implant + sodium acetate |
| | buffer |
| 3 | Implant + 15 μg rhPDGF-BB |
| 4 | Implant + 75 μg rhPDGF-BB |
| 5 | Implant + 500 μg rhPDGF-BB |

### Surgical Protocol

### 1) Animal Anesthesia and Surgical Preparation

One (1) osteochondral defect was created in the right medial femoral condyle. The basic surgical procedure was identical for all subjects. All surgeries were performed under strict asepsis. All animals will have food and water removed 12 hours before surgery.

An IV injection consisting of Diazepam 0.22 mg/kg and Ketamine 10 mg/kg were given for induction of general anesthesia. A cuffed endotracheal tube was placed and general anesthesia was maintained with Isoflurane 0.5-5% delivered through a rebreathing system. One 50 μg Fentanyl patch was applied to the tail just prior to surgery for approximately 72 hours of post operative pain. Xylazine aided in analgesia during the acute post operative time. Each knee was physically examined for drawer range of motion (goniometer), swelling, temperature, crepitus, patella tracking, and valgus/varus. A physical examination record was provided by Applied Biological Concepts for each animal at the time of surgery.

The animal was then transferred to the operating suite and positioned in dorsal recumbency. The endotracheal tube was attached to an anesthesia machine delivering oxygen, room air and Isoflurane. The surgical area was shaved and prepped. A standard surgical scrub with chlorohexidine detergent followed by wash with 70% alcohol and followed with a paint of betadine was performed. Each animal will receive peri-operative antibiotics for prophylaxis. Maintenance of a surgical plane of anesthesia was achieved by inhalation anesthesia using Isoflurane (range 0.5-5.0% depending on animal) and oxygen (1.5 L/min). While the animal was under anesthesia the heart rate, respiratory rate and mucus membranes were monitored a minimum of every 15 minutes.

### 2) Blood Collection

In addition to blood collection for pre-surgery and pre-necropsy health screening, one extra tube of blood was collected the day of surgery and the day of euthanasia in a clot tube and the serum collected, and at least 2 ml of serum placed into a cryovial labeled with the study number, animal number, and collection date and stored frozen at −70 to −80° C.

### 3) Synovial Fluid Collection

At the time of surgery, for all animals, gross evaluation of the synovial fluid for color and viscosity were recorded. If sufficient volume permits, a sample of the synovial fluid was collected and placed into a cryovial labeled with the study number, animal number, collector's initials, and collection date and stored frozen at −70 to −80° C.

At the time of necropsy, gross evaluation, the color and viscosity of the synovial fluid were recorded. For all animals, gross evaluation of the synovial fluid for color and viscosity were recorded. If sufficient volume permits, up to a 500 μl sample of the synovial fluid were collected and placed into a cryovial labeled with the study number, animal number, and collection date, and stored frozen at −70 to −80° C.

### 4) Surgical Implantation

The surgical approach consisted of a curved, lateral skin incision made from the distal one-third of the right femur to the level of the tibial plateau and across to the medial side of the tibial spine. Using this method, the skin was bluntly dissected and retracted to allow a lateral parapatellar approach into the stifle joint. An incision was made parallel to the lateral border of the patella and patellar ligament. This extended from the lateral side of the fascia lata along the cranial border of the biceps femoris and into the lateral fascia of the stifle joint. The biceps femoris and attached lateral fascia were retracted allowing an incision into the joint capsule. The joint was extended and the patella luxated medially exposing the stifle joint.

With the knee joint fully flexed, the fat pad may be partially dissected with cautery to allow visualization of the medial femoral condyle. The point of drilling for the medial femoral condyle was defined as 19 mm distal to the condyle groove junction and aligned with the medial crest of the trochlear groove. Using the surgical instruments supplied, an 8 mm diameter by 8 mm deep osteochondral defect was created. The defect was copiously flushed with sterile saline. The remaining portions of the joint were carefully flushed prior to placement of the test article, and the joint blotted dry before placement of any test article.

The medial femoral condylar defect was either left empty (Group 1) or filled with a Chondromimetic implant that had been saturated with either the control 20 mM sodium acetate solution (Group 2) or one of the 3 dosages of rhPDGF-BB (Groups 3-5).

The patella was then reduced. This was followed by routine closure of the joint in three layers using 1 Vicryl suture material and surgical skin staples. Following closure of the surgical incision, a modified Thomas splint was applied to the leg to limit weight bearing and motion. The fiberglass cast and splint remained on for 14 days post-operatively. For splint removal, the animals were given an IV injection consisting of Diazepam 0.22 mg/kg and Ketamine 10 mg/kg for induction of short-term, general anesthesia. While anesthetized, the splint was removed. The leg was not moved through a full range of motion.

Digital photographs of each surgical site following implantation of test or control articles were taken for each animal. The animal number and date were included on the digital photograph.

### 5) Materials Preparation

Bi-phasic Matrix Implant/rhPDGF-BB:

Prior to implantation, one of three doses of rhPDGF-BB (0.030 mg/ml, 0.15 mg/ml, or 1.0 mg/ml rhPDGF-BB in buffer) or 20 mM sodium acetate buffer, was combined with the bi-phasic collagen implant by adding 0.5 cc of the appropriate test article to the sterile, collagen implant in a stainless steel bowl. The hydrated collagen implant was allowed to sit at room temperature for 5-15 minutes and then gently transferred with surgical forceps to the defect site. Any excess rhPDGF-BB solution was drawn up by syringe and expressed into the defect site.

### In-Life Observations and Measurements

A minimum of twice-daily postoperative checks were made for any animal displaying signs of postoperative discomfort. Additional postoperative analgesics were given if the animals display any signs of distress of discomfort. All treatments were recorded in the appropriate study documentation. Daily clinical observations were performed and recorded for each animal until the time of euthanasia. Post-op checks were done on all animals as part of routine clinical observations.

Bodyweight measurements were taken from all animals prior to surgery (Day 0) and at the end of the study (Week 12±0.5). Food consumption was qualitative. Animals were monitored daily and the degree of appetite was recorded.

### Necropsy

Animals were humanely sacrificed at Day 84±3 (12 weeks) postoperatively. Bodyweights were recorded immediately prior to sacrifice. An IV injection consisting of Diazepam 0.22 mg/kg and Ketamine 10 mg/kg was given for induction of general anesthesia. Following this, the anesthetized animals were given an IV overdose of concentrated potassium chloride (KCl) until the cardiac arrest has been verified.

At the time of euthanasia, blood was taken from each animal. Each knee was physically examined for drawer, range of motion (goniometer), swelling, temperature, crepitus, patella tracking, and valgus/varus. A physical examination record was provided by Applied Biological Concepts for each animal at the time of necropsy.

The stifle joints were grossly evaluated, synovial fluid evaluated grossly for color and viscosity, and samples collected as described in Table 12. The joints were opened, photographed and the surface of the osteochondral defect sites scored as indicated in Table 13. The articulating surfaces opposing the defect sites were examined for any abnormal joint surface.

Gross evaluations were performed on the control and operated knee joints. Gross evaluation included scoring of edge integration of nascent repair tissue relative to native cartilage, smoothness of repair surface, degree of fill, and the color of the repair tissue.

Popliteal lymph nodes and the synovial membranes were examined for any lymphadenopathy and inflammation, respectively.

TABLE 12
Gross Evaluation and Sample Collection

| | Gross | Photograph | Sample | Sample | |
|---|---|---|---|---|---|
| Sample | Evaluation | and Score | collection | histology | Initials |
| 2 ml serum - time zero | | | X | | |
| 2 ml serum - at necropsy | | | X | | |
| Right Popliteal lymph | X | | X | X | |
| node | | | | | |
| Right Knee joint Synovial | X | | X* | | |
| Fluid | | | | | |
| Right Knee joint | X | | | | |
| Right Femur | X | X | X | | |
| Right Femur Implant | | | | X | |
| Sites (submitted | | | | | |
| individually) | | | | | |
| Left Popliteal lymph node | X | | X | X | |
| Left Knee joint | X | | | | |
| Left Knee joint Synovial | X | | X* | | |
| Fluid | | | | | |
| Left Femur | X | X | X | | |

X* = sample saved if volume permits

TABLE 13
Scoring Criteria for Gross Morphological Evaluations

| Characteristic | Grading | Score |
|---|---|---|
| Edge Integration | Full | 2 |
| (new tissue relative to native cartilage) | Partial | 1 |
| | None | 0 |
| Smoothness of the cartilage surface | Smooth | 2 |
| | Intermediate | 1 |
| | Rough | 0 |
| Cartilage surface, degree of filling | Flush | 2 |
| | Slight depression | 1 |
| | Depressed/overgrown | 0 |
| Color of cartilage, opacity or | Opaque | 2 |
| translucency of the neocartilage | Translucent | 1 |
| | Transparent | 0 |

### Histologic Analysis

Immediately after gross evaluation, the specimens, along with the left and right popliteal lymph nodes, were placed in 10% phosphate buffered formalin (at least ten-fold volume). The formalin fixed specimens were grossly trimmed to remove extra tissue. The popliteal lymph nodes were processed using standard histological techniques as known by one skilled in the art and stained with hematoxylin and eosin (H&E). These soft tissues were graded for inflammation, fibrosis, or other changes according to the following grading system:
0=No Change
1=Minimal Change
2=Mild Change
3=Moderate Change
4=Marked Change

The femoral specimens were then decalcified in 10% EDTA or Formic Acid until complete decalcification was determined. Contact radiographs were taken prior to decalcification to ensure complete decalcification of the sample. Following complete decalcification, the specimens were dehydrated through a series of ethanol exchanges of increasing concentrations, if necessary a xylene or other appropriate chemical exchange were done to remove excess fat in the specimen and improve penetration into the specimen, and the specimen embedded in paraffin. Four sections, 5-10 μm thick were made. One section was stained with hematoxylin and eosin (H&E). The second section was stained with Safranin O and counterstained with Fast Green. A third and fourth section were made and these sections will undergo immunohistochemical staining for Type I and Type II collagen. Two additional slides were also made and left unstained.

### Synovial Fluid Evaluation

Gross evaluation of the synovial fluid for color and viscosity were recorded. A synovial fluid sample was saved from each knee joint. Synovial fluid were stored in a labeled cryovial and stored at −70 to −80° C.

### MicroCT Analysis

MicroCT scanning and analysis was performed on a microCT80 system (SCANCO USA, Southeastern, Pa.) using the manufacturer's analysis software. Endpoints for microCT analysis will include assessment of bony fill throughout the subchondral zone and the bone volume/total volume (BV/TV) of the central cavity.

Osteochondral defect site gross morphological evaluations were summarized for each treatment group on the basis of the individual characteristic scores and on the total score. Nonparametric tests were used to compare the treatment groups that fit the data with a significance level of p<0.05. Histological change scores were similarly evaluated.

### Results

### Gross Necropsy

Animals were observed twice daily until sacrifice. All animals were sacrificed 12 weeks following surgery and specimens were collected for microCT and histological analysis.

All animals exhibited moderate healing of the defect site unless otherwise noted:

3379 had incomplete filling of the defect.
3743 had visible blood spots within defect, healing site not flush with surrounding cartilage.
3743 had blood spots observed in defect site, vascularization of the fossa in the right stifle joint, defect in right and left patellofemoral groove.
3382 had visible blood spots within defect.
3388 had visible blood spots, darker tissue color, and irregular surface to the repair tissue within the defect site.
3733 had visible blood spots, and a minor osteophyte on medial condyle (medial to defect location).
3383 had visible blood spots, good healing at defect site, and calcinosis on posterior medial condyle of right stifle joint.
3375 had poor filling of the defect, very little soft tissue formation, repair tissue appeared dull and darkened, with collapse observed at defect site, osteophyte formation on medial and lateral condyles of the right stifle joint, small osteophyte formation on the lateral condyle of the left stifle joint, and fibrosis in the fat pad of the right stifle joint.
3387 had an irregular defect surface.
3728 had poor filling of the defect, very little overlying soft tissue formation, where color appears dull and darkened, focal defect on posterior aspect of medial condyle (˜20 mm from defect) on both left and right stifle joints, and cartilage damage in patellofemoral groove of both right and left stifle joints.
3735 had visible blood spots, a slight depression at healing site, and osteophyte formation in the medial aspect of proximal medial tibia of right stifle joint.
3746 had visible blood spots in repair tissue, and slight osteophyte formation medial and lateral of defect.
3749 had visible blood spots in repair tissue, osteophyte formation on the patellar groove, patella, condyle and patellofemoral groove of the left stifle joint, and hyperemia in the fat pad of the right stifle joint.
3393 had visible blood spots in the repair tissue.
3737 had visible blood spots in the repair tissue, depression in the repair tissue, and small osteophyte formation medial of the defect.
3731 had visible blood spots in the repair tissue, a slight depression of the surface of the repair tissue, and some roughness of the cartilage surface proximal to the defect.
3384 had good healing, repair tissue flush with the cartilage surface, and visible blood spots in the repair tissue.
3742 had poor filling of the defect, very little soft tissue formation that appeared dull and darkened in color.
3376 had visible blood spots in the repair tissue.
3747 had visible blood spots in the repair tissue, good healing of the defect, diameter of the defect decreased to 3-4 mm, and small osteophyte formations on the medial and lateral aspects of the medial femoral condyle of the right stifle joint.
3748 had visible blood spots in the repair tissue, healing site depressed compared to surrounding cartilage, right stifle joint synovial fluid was hazy, laxity in right stifle joint where the anterior cruciate ligament was stretched out but not torn, fat pad hyperemia in right stifle joint, synovial fluid of left stifle joint was clear, and cartilage wear on the proximal aspect of the medial condyle and tibial plateau of the right and left stifle joints.
3734 had significant visible blood spots in repair tissue, slight depression at repair site, and small osteophyte formation on lateral aspect of medial femoral condyle of the right stifle joint.
3732 had visible blood spots in the repair tissue, and repair site was slightly depressed compared to surrounding cartilage.
3390 had significant blood spots visible in repair tissue.
3739 had blood spots visible in repair tissue, and an irregular surface to the repair tissue.
3738 had visible blood spots in the repair tissue, defect site was flush with defect rim, osteophyte formation medial and lateral to defect site, and the medial meniscus of the right stifle joint had visible vascular changes.
3736 had visible blood spots in the repair tissue.
3730 had visible blood spots in the repair tissue, and repair tissue was flush with surrounding cartilage surface.
3745 had very little overlying soft tissue formation, no bone collapse, and the repair tissue within the defect was pink in color.
3389 had repair tissue with an irregular surface.
3380 had poor filling of the defect, very little soft tissue formation with color that appeared dull and dark, a collapse of the repair was observed at the defect site, and hyperemia located in the fat pad of both right and left stifle joint.

### Gross Evaluation

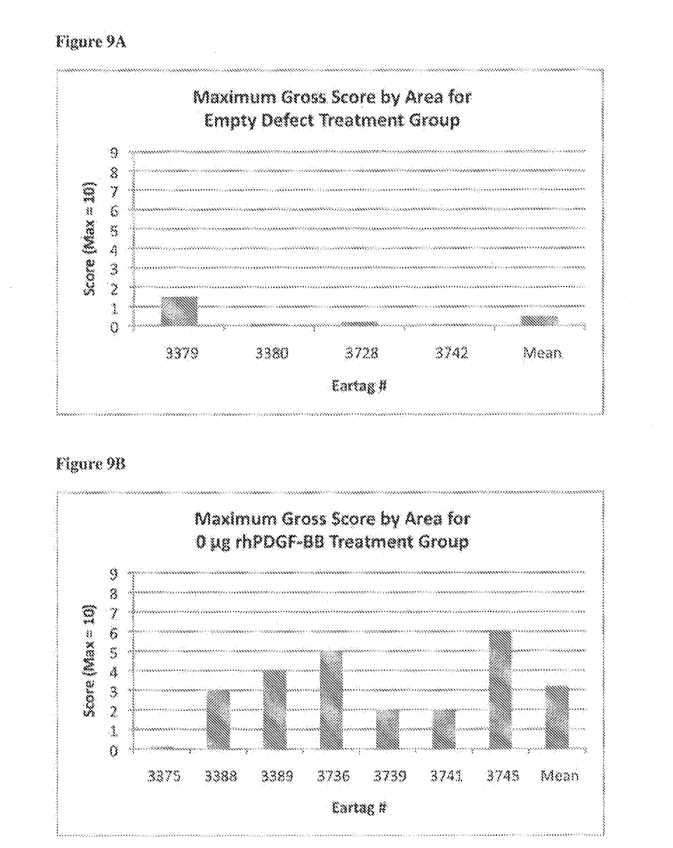

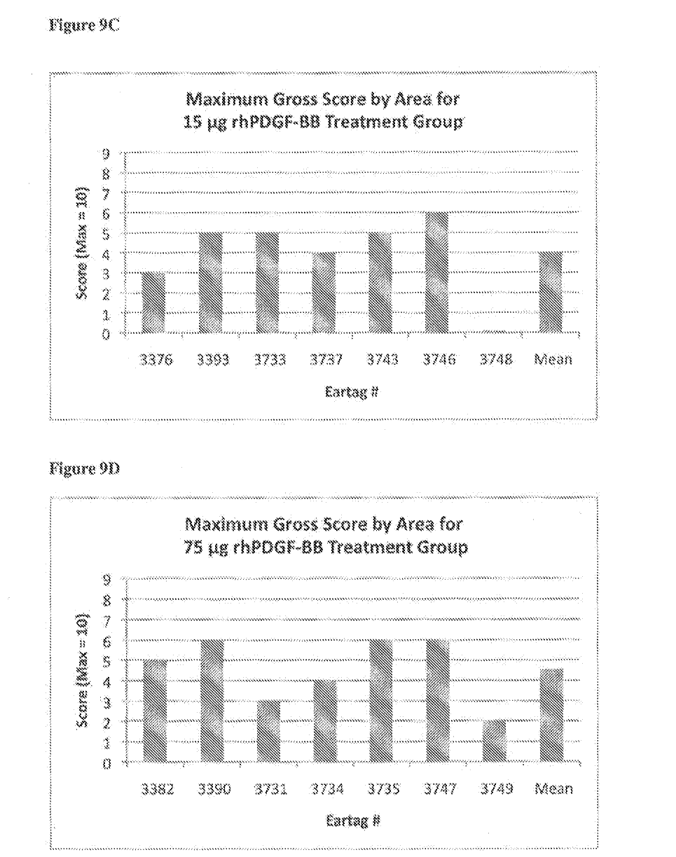

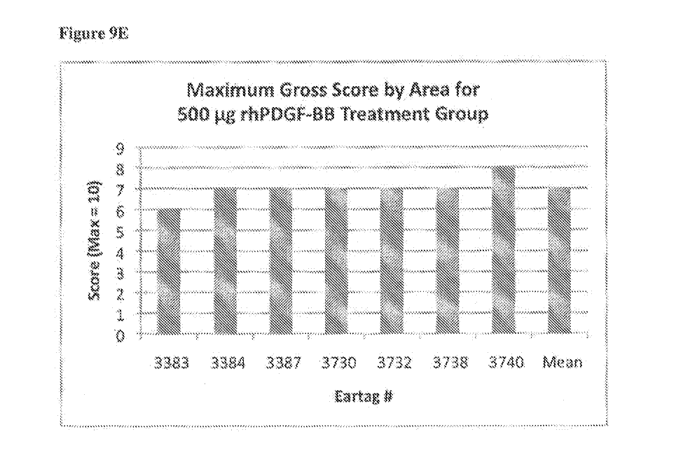

Maximum gross score by area for each specimen within each treatment group were presented in FIGS. 9A to 9E. For scoring criteria, see Scoring Criteria for Gross Morphological Evaluation (Table 13). A one-way ANOVA with a Tukey post-hoc test was performed in GraphPad Prism 5 to determine the effect of the treatment group on the quantitative measures. Significance was determined at p<0.05.

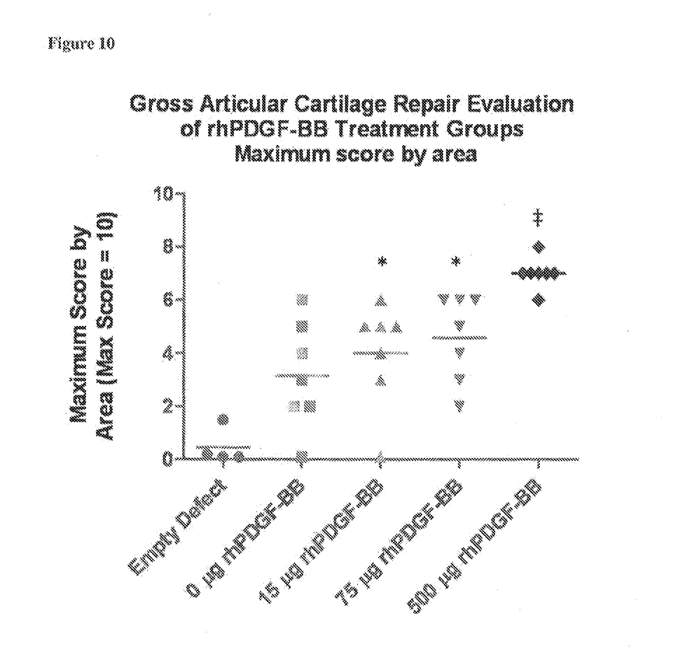

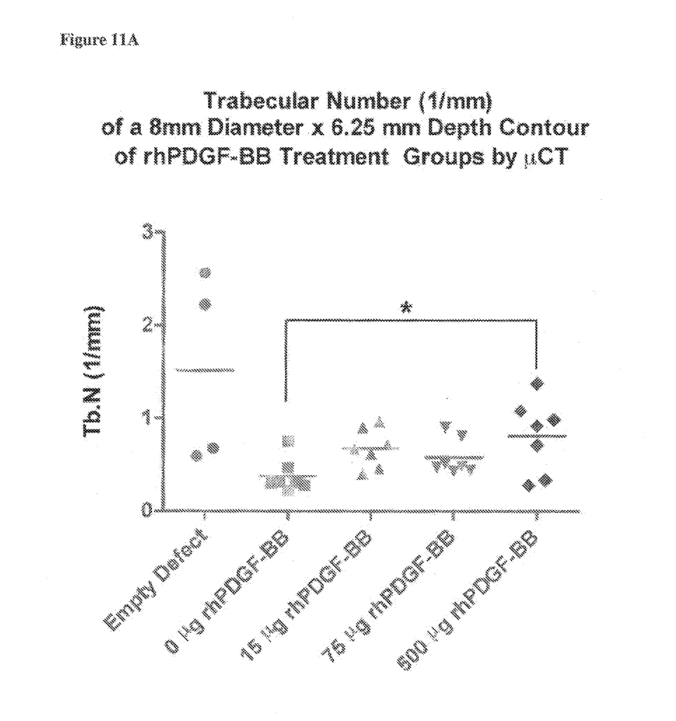

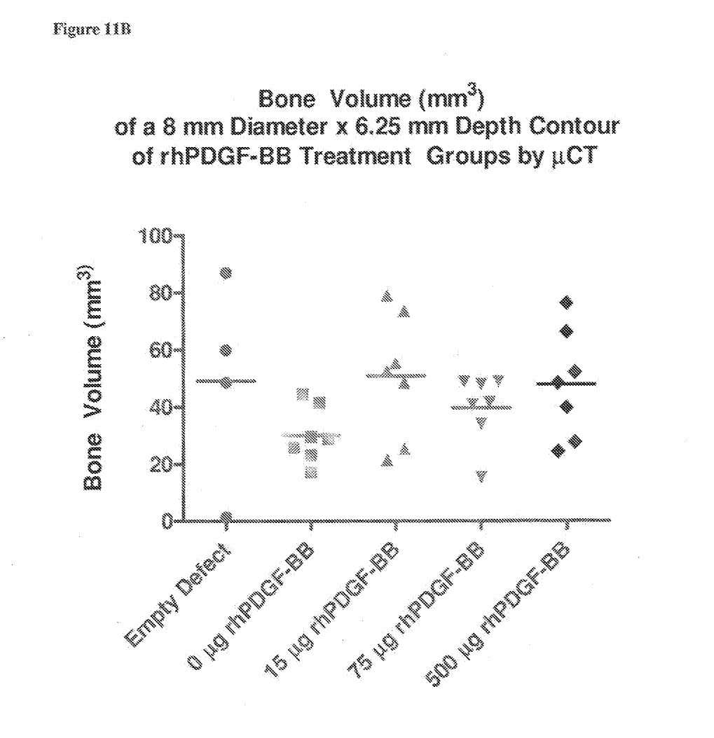

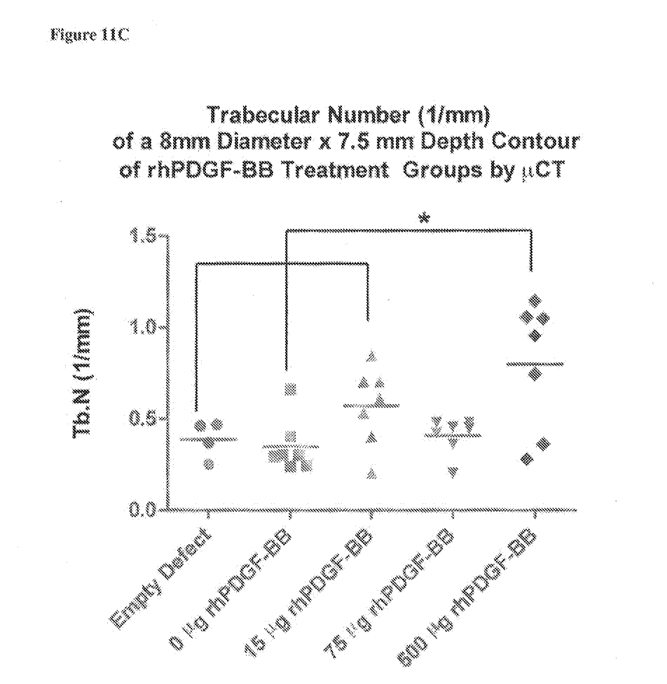

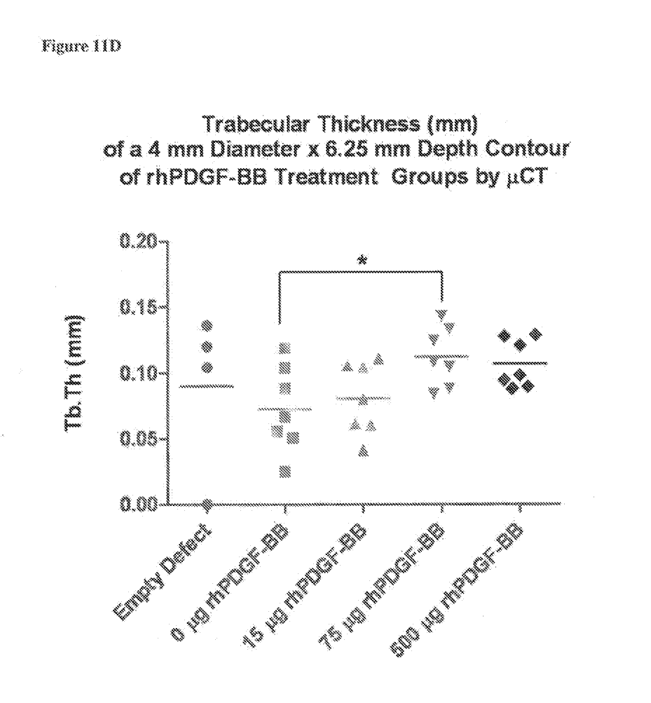

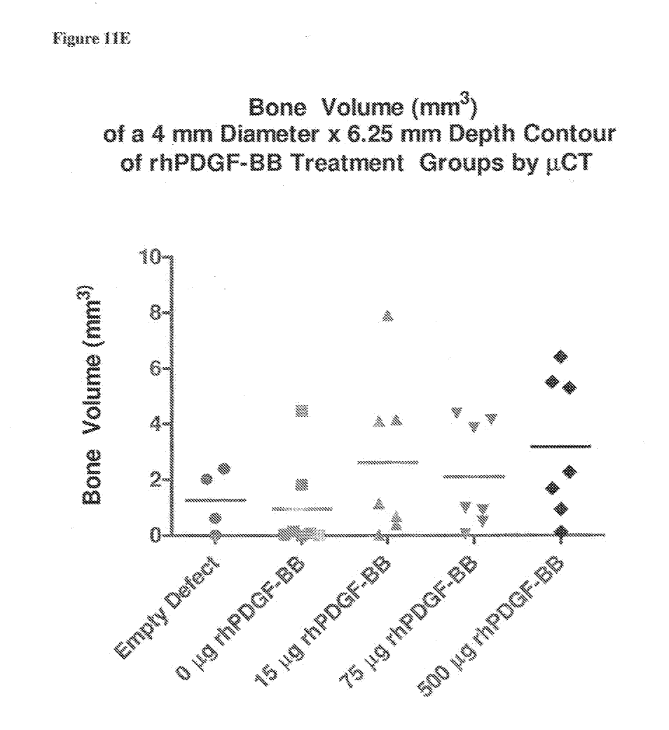

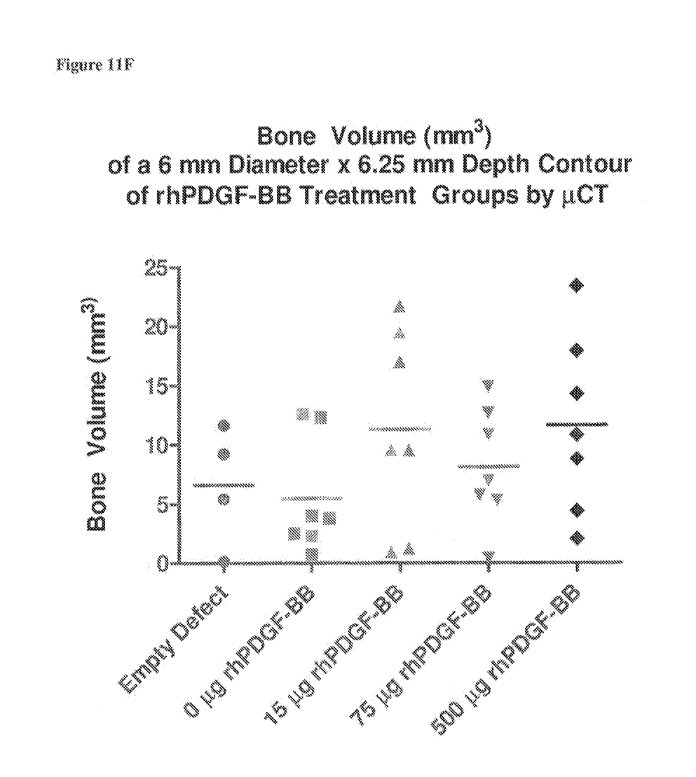

The maximum gross score by area was significantly increased (FIG. 10) in specimens treated in either the 500 μg rhPDGF-BB, 75 μg rhPDGF-BB, or 15 μg rhPDGF-BB treatment groups compared to specimens in the Empty Defect treatment group. Additionally, there was a significant increase in maximum score by area for the 500 μg rhPDGF-BB compared to the 15 μg rhPDGF-BB and 0 μg rhPDGF-BB treatment groups.

### MicroCT

Animals were humanely euthanized and operated (right) stifle joints were harvested, placed in 10% neutral buffered formalin for microCT analysis. In addition to the operated stifle joints, all unoperated contralateral (left) stifle joints were harvested for microCT analysis. The description of microCT is presented in Example 8.

Animals were humanely euthanized and operated stifle joints were harvested in 10% neutral buffered formalin. Following microCT analysis, all samples had a fresh 10% neutral buffered formalin exchanged, and were ready for histological preparation. All specimens were processed and paraffin embedded for undecalcified histologic analysis. The coronal aspect was processed for decalcified histology and embedded in paraffin. The specimens were fixed, decalcified, dehydrated, cleared, infiltrated, and embedded using standard paraffin histology techniques and equipment. Six sections were taken at 3-5 μm steps: two unstained sections were not tested, two sections were prepared for IHC (immunohistochemical) analysis for Type I and Type II collagen, one section was stained with safranin-o and fast green, and the final section was stained with H&E. Both stain sections and sections prepared for IHC analysis were ready for histopathology.

TABLE 14
Specimens Allocated for Histological Evaluation

| Treatment | Sample Size |
|---|---|
| Empty Defect | 4 |
| Implant + 0 μg rhPDGF-BB | 7 |
| Implant + 15 μg rhPDGF-BB | 7 |
| Implant + 75 μg rhPDGF-BB | 7 |
| Implant + 500 μg rhPDGF-BB | 7 |

### Histology

All decalcified tissue sections were graded according to the Modified Sellers Scoring System. Evaluations included the nature of the predominant tissue, structural characteristics, freedom from cellular changes of degeneration, freedom from degenerative changes in adjacent cartilage tissue, reconstitution of subchondral bone, and intensity of safranin-o staining. Sections were first assessed and evaluated for overall healing compared to one another and given a healing score. The histologic scoring scale for cartilage and bone repair is listed in Table 15 below.

TABLE 15
Histologic Scoring Scale for Cartilage and Bone Repair

| Characteristic | | Grading | Score |
|---|---|---|---|
| I. | Nature of predominant tissue | hyaline cartilage | 4 |
| | (indication of dose-dependent | mostly hyaline cartilage | 3 |
| | increase in hyaline like | mixed hyaline and fibrocartilage | 2 |
| | cartilage formation) | mostly fibrocartilage | 1 |
| | | some fibrocartilage, mostly | 0 |
| | | nonchrondocytic cells | |
| II. | Structural Characteristics | | |
| | (indication of dose-dependent | | |
| | preservation of native | | |
| | cartilage and repair tissue | | |
| | surface integrity/integrity with | | |
| | rhPDGF-BB) | | |
| | A. Surface regularity | smooth and intact | 3 |
| | | superficial horizontal lamination | 2 |
| | | fissures | 1 |
| | | severe disruption, including fibrillation | 0 |
| | B. Structural Integrity | normal | 2 |
| | | slight disruption, including cysts | 1 |
| | | severe disintegration | 0 |
| | C. Thickness | 100% of normal adjacent cartilage | 2 |
| | | 50-100% of normal cartilage | 1 |
| | | 0-50% of normal cartilage | 0 |
| | D. Bonding to adjacent | Bonded at both ends of graft | 2 |
| | cartilage | Bonded at one end or partially at both ends | 1 |
| | | Not bonded | 0 |
| III. | Freedom from Cellular | | |
| | Changes of Degeneration | | |
| | (Indication of hypercellularity | | |
| | in rhPDGF-BB treated groups | | |
| | as compared to biphasic | | |
| | biocompatible matrix plug | | |
| | alone or empty defect). | | |
| | A. Hypocellularity | normal cellularity | 2 |
| | | slight hypocellularity | 1 |
| | | moderate hypocellularity or | 0 |
| | | hypercellularity | |
| | B. Chondrocyte Clustering | No clusters | 2 |
| | | <25% of the cells | 1 |
| | | 25-100% of the cells | 0 |
| IV. | Freedom from Degenerative | Normal cellularity, no clusters, normal | 3 |
| | Changes in Adjacent | staining | |
| | Cartilage (Indication of | Normal cellularity, mild clusters, moderate | 2 |
| | decreased degenerative | staining | |
| | changes in adjacent cartilage | Mild or moderate hypocellularity, slight | 1 |
| | tissues as compared to empty | staining | |
| | defect) | Severe hypocellularity, poor or no staining | 0 |
| V. | A. Reconstitution of | Normal | 3 |
| | subchondral | Reduced subchondral bone reconstitution | 2 |
| | Bone (Indication of dose- | Minimal subchondral bone reconstitution | 1 |
| | dependent increase in | No subchondral bone reconstitution | 0 |
| | Safranin-O staining with | | |
| | rhPDGF-BB) | | |
| | B. Inflammatory response in | None/mild | 2 |
| | subchondral bone region | Moderate | 1 |
| | (indication of non/mild | Severe | 0 |
| | inflammation in all treatment | | |
| | groups as compared to empty | | |
| | defect) | | |
| VI. | Safranin-O Staining | Normal | 3 |
| | (indication of dose-dependent | Moderate | 2 |
| | increase in Safranin-O | Slight | 1 |
| | staining with rhPDGF-BB). | None | 0 |

| | |
|---|---|
| TOTAL MAXIMUM SCORE: | 28 |

*If the tissue is scored as “4 = hyaline cartilage” it essentially consists of only hyaline cartilage, no trace of fibrocartilage. Scoring the nature of the repair tissue as “3 = mostly hyaline cartilage” is given to sections which have some trace of fibrocartilage, but less than 25% as determined visually. A score of “2 = mixed hyaline and fibrocartilage” is given to repair tissue which has both hyaline and fibrous tissue, varying from approximately75% hyaline/25% fibrous to 25% hyaline/75% fibrous. A score of “1 = mostly fibrocartilage” is given to repair tissue which showed some traces (less than 25%) of hyaline, but was primarily fibrous in nature. A score of “0 = some fibrocartilage, mostly non-chondrocytic” is given to repair tissue which does not exhibit any hyaline tissue at all.

The results show the following: 1) minimal inflammatory response for all treatment groups; 2) dose-dependent increase in histological repair total score for rhPDGF-BB treatment groups compared to 0 μg rhPDGF-BB treatment group or empty defect treatment group; 3) dose-dependent increase in reconstitution of subchondral bone for PDGF treatment groups compared to 0 μg rhPDGF-BB treatment group or empty defect treatment group; 4) dose-dependent increase in the number and/or thickness of nascent bony trabeculae within the defect space for PDGF treatment groups compared to 0 μg rhPDGF-BB treatment group or empty defect treatment group; 4) newly formed trabeculae primarily isolated to the base and edges of the defect, with the exception of a number of specimens within the 500 μg rhPDGF-BB treatment group, where bridging of the defect space is noted; 5) incomplete filling of the defect, and/or collapse of surrounding native cartilage into the defect, in the empty defect treatment group; 6) dose-dependent integration of repair tissue with adjacent native cartilage for PDGF treatment groups compared to 0 μg rhPDGF-BB treatment group or empty defect treatment group; 7) repair tissue consisting primarily of fibrocartilage for all treatment groups; 8) presence of hyaline like cartilage, indicated by positive immunohistochemistry staining for Type II collagen and glycosaminoglycan content as determined by Safaranin-O staining, in central region of the chondral zone of the defect for all treatment groups with the exception of the empty defect treatment group; and 9) small amount of residual collagen implant within the defect space for all treatment groups containing the collagen implant.

Additionally, quantitative histomorphometric analysis is completed to further evaluate the tissue filling the defect. Total area of the repair tissue and the percentages of the specific tissues present (hyaline cartilage, fibrocartilage, fibrous tissue, osseous tissue) are evaluated. The results include the following: 1) an increase in the percentage of reconstitution for the subchondral space by calcified tissues (new bone) in rhPDGF-BB treatment groups compared to 0 μg rhPDGF-BB treatment group or empty defect treatment group; 2) dose-dependent increase in total fill of the defect by all tissues for rhPDGF-BB treatment groups compared to 0 μg rhPDGF-BB treatment group or empty defect treatment group; 3) dose-dependent increase in the percentage of hyaline-like cartilage within the chondral region of the defect space for rhPDGF-BB treatment groups compared to 0 μg rhPDGF-BB treatment group or empty defect treatment group; 4) dose-dependent increase in the percentage of fibrocartilage within the chondral region of the defect space for rhPDGF-BB treatment groups compared to 0 μg rhPDGF-BB treatment group or empty defect treatment group; and 5) decrease native cartilage tissue collapse into the defect space for all treatment groups containing a collagen implant (0, 15, 75, 500 μg rhPDGF-BB) compared to the empty defect treatment group.

### Example 8

### MicroCT Evaluation of Subchondral Bone Reconstitution for Osteochondral Defect Repair in a Caprine Model

The objective of the study was to assess the degree of subchondral bone repair of caprine femoral condyles in an osteochondral defect model. Quantitative factors (e.g., bone volume, trabecular thickness, etc.) were measured to evaluate the quantity and quality of bone formed. The treatment groups used in this study were the same as the ones used in Example 7.

### Materials and Methods

Scanning Protocol for Medial Femoral Condyle in the microCT

Each medial femoral condyle in the 51.2 mm brown resin specimen holder was loaded. Each condyle was then wrapped tightly in foam rubber to stabilize it in the specimen holder. The wrapped condyle was inserted into the specimen tube with the defect side facing up and it was parallel with the long axis of the tube. The stability of the condyle was checked by rotation and movement of the specimen side-to-side within the tube. After loading and checking the stability of each condyle, 10% neutral buffered formalin was added to completely submerge the specimens while leaving 2-3 mm of air at the top of the tube. The specimen tube was sealed with the plastic tube cap. The sealed specimen tube in the microCT with the orientation scratch was placed facing the user.

The scans of the condyle were then acquired and the Evaluation software was then launched. Specimen slices were contoured by hand drawing a region of interest in a counter-clockwise motion that closely approximated the outer surface of the entire condyle.

Quantitative analysis of the specimen was then carried out. For the 560 slices in the condyle region, 250 slices (6.25 mm depth) or 300 slices (7.5 mm depth) were contoured, starting with the first full slice that entirely outlined the original defect. The remodeled defect site was contoured by drawing a circular contour of the following dimensions for each analysis: 160 pixels×160 pixels (0.1266 cm², 4 mm diam.), 240 pixels×240 pixels (0.2842 cm², 6 mm diam.), 320 pixels×320 pixels (0.5046 cm², 8 mm diam.), 400 pixels×400 pixels (0.785 cm², 10 mm diam.), centered on the central canal of the original defect.

A one-way ANOVA with a Tukey post-hoc test was performed in GraphPad Prism 5 to determine the effect of the treatment group on the quantitative measures. Significance was determined at p<0.05.

### Results

Quantitative measures of the total volume, bone volume, material mean density, connectivity density, trabecular number, trabecular thickness, and trabecular separation were evaluated using the analysis program of the Scanco microCT80 machine (Southeastern, Pa.). The treatment groups and number of animals per group were the same as the ones used in Example 7 and outlined in Table 16. The quantitative analysis was performed on the central canal of the original defect using multiple analysis criteria, including: 8 mm diameter cylinders which were 6.25 mm in depth, 6 mm diameter cylinders which were 6.25 mm in depth, 4 mm diameter cylinders which were 6.25 mm in depth, 8 mm diameter cylinders which were 7.5 mm in depth, and 10 mm diameter cylinders which were 6.25 mm in depth. The total volume (volume of the contoured cylinder) was kept constant for each analysis criteria. No significant differences were observed for the connectivity density or trabecular separation. For all analysis criteria, no significant differences in bone volume between treatment groups were noted, however substantial bony bridging spanning the entire width of the defect space was noted in four out of seven specimens for the 500 μg rhPDGF-BB treatment group. This type of bridging was not observed in remaining treatment groups. The trabecular number (FIG. 10A) of the specimens treated with 500 μg rhPDGF-BB were significantly increased compared to the 0 μg rhPDGF-BB treatment group for the 8 mm thickness×6.25 mm depth contour. The trabecular number (FIG. 10C) was significantly increased in the 500 μg rhPDGF-BB treatment group compared to the 0 μg rhPDGF-BB, 15 μg rhPDGF-BB, and Empty Defect treatment groups for the 8 mm diameter×7.5 mm depth contour. Trabecular thickness (FIG. 10D) was significantly increased in specimens treated with 75 μg rhPDGF-BB compared to the 0 μg rhPDGF-BB treatment group for the 4 mm thickness×6.25 mm depth contour. There was also a significant increase in the trabecular thickness of the Empty Defect treatment group compared to the 0 μg rhPDGF-BB, 75 μg rhPDGF-BB, and 500 μg rhPDGF-BB (10 mm diameter×6.25 mm depth contour), and the 0 μg rhPDGF-BB, 15 μg rhPDGF-BB, and 75 μg rhPDGF-BB treatment groups (8 mm diameter×7.5 mm depth contour).

TABLE 16
Treatment Groups

| | | Implant | | | |
|---|---|---|---|---|---|
| | Implant | (Chondromimetic | Growth | Survival | # |
| Group | Site | type I collagen) | Factor | (weeks) | Animals |
| 1 | MFC | No | None | 12 ± 0.5 | 4 |
| 2 | MFC | Yes | NaAc | 12 ± 0.5 | 7 |
| | | | Buffer | | |
| 3 | MFC | Yes | rhPDGFBB | 12 ± 0.5 | 7 |
| | | | 15 μg | | |
| 4 | MFC | Yes | rhPDGFBB | 12 ± 0.5 | 7 |
| | | | 75 μg | | |
| 5 | MFC | Yes | rhPDGFBB | 12 ± 0.5 | 7 |
| | | | 500 μg | | |

| | |
|---|---|
| Total | 32 |

MFC: Medial Femoral Condyle

### CONCLUSION

Several points of analysis revealed a significant increase in trabecular thickness for the Empty Defect treatment group compared to remaining treatments. This difference may be attributed to the collapse of surrounding endogenous bone into the defect space for the Empty Defect treatment group, where the endogenous bone was analyzed within the contours compared to nascent bone in remaining treatment groups with no collapse of the defect profile. The significant increases in trabecular number found in the 500 μg rhPDGF-BB treatment group for multiple contour analyses, coupled with the presence of enhanced bony bridging compared to other treatment groups, would indicate rhPDGF-BB has a significant impact on new bone formation at the early time point of 12 weeks. This enhancement of subchondral bone reconstitution could result in an augmented repair, suggesting that when combined with a collagen matrix, rhPDGF-BB may have promise as a therapeutic treatment for osteochondral defect repair.

### Example 9

### A Pilot Human Clinical Trial to Evaluate the Safety of Using Biphasic Biocompatible Matrix and rhPDGF-BB for Surgical Repair of Osteochondral Defects in the Knee

The primary objective of the study is to confirm the safety and explore the performance of rhPDGF-BB and biphasic biocompatible matrix (e.g., Chondromimetic (Orthomimetics)) for treatment of high-load-bearing and low-load-bearing osteochondral defects of the knee. The secondary objective of the study is to evaluate the surgical procedure and clinical outcome measurements (ICRS—International Cartilage Repair Society Form, VAS—Visual Analogue Scale, Cincinnati Rating, KOOS-Knee Injury and Osteoarthritis Outcome Score) for the implantation of rhPDGF-BB and biphasic biocompatible matrix.

The study is carried out in 3 clinical centers. In each clinical center, the study includes 3 groups of qualified subjects. The qualified subject (human) meets the inclusion criteria listed in Table 17. The first group (Group 1 (control)) consists of six qualified subject without bone and/or cartilage defects caused by trauma (e.g., sports injuries) or without early stage osteochondral defects and receives no treatment. The control group can also be based on historical controls, based on published articles, as known by one skilled in the art. The second group (Group 2) consists of seven qualified subjects with at least one osteochondral defect (<12 mm) to the knee that requires surgical treatment by either minimally invasive or open procedure. This group receives biphasic biocompatible matrix plug (e.g., Chondromimetic (Orthomimetics)) and 500 μg rhPDGF (0.5 cc 1.0 mg/ml rhPDGF-BB) per defect placed in a low-load-bearing region of the knee, with a maximum of 6 defects per qualified subject. The third group (Group 3) consist of seven qualified subjects with at least one osteochondral defect (<12 mm) to the knee that requires surgical treatment by either minimally invasive or open procedure. This group receives biphasic biocompatible matrix plug (e.g., Chondromimetic (Orthomimetics)) and 500 μg rhPDGF (0.5 cc 1.0 mg/ml rhPDGF-BB) per defect placed in a high-load-bearing region of the knee.

TABLE 17
Inclusion/Exclusion Criteria:

Inclusion:
Review, understand, and sign informed consent
At least one osteochondral defect (<12 mm) to the knee (Orthomimetic
states <12 mm)
Independent, ambulatory, and can comply with all post-operative
evaluations and visits.
At least 18 years of age and considered to be skeletally mature
Symptoms must include pain, pain with weight bearing and squatting,
locking of joints or swelling.
Exclusion:
Index knee has undergone previous treatment for cartilage repair with
ACI, osteochondral grafting, microfracture, and/or autologous
chondrocytes.
Ligament treatment within the affected knee within one year prior to
current study
Treatment of meniscal pathology within the affected knee by partial or
total meniscectomy within six months prior to current study
Mechanical axis malalignment of greater than 5 degrees
Presence of inflammation or osteoarthritis of the knee
Infection in the knee
The patient currently has untreated malignant neoplasm(s), or is currently
undergoing radio- or chemotherapy.
Has a pre-existing sensory impairment which limits the ability to perform
objective functional measurements
Physically or mentally compromised.
Allergy to yeast-derived products.
Has received an investigational therapy or an approved therapy for
investigational use within 30 days of surgery or during the follow-up
phase of this study.
Prisoner, or is known or suspected to be transient.
History of drug/alcohol abuse within 12 months prior to screening.
Pregnant or a female intending to become pregnant during this study
BMI (body mass index) is greater than 40 kg/m².
Acute infection at the surgical site.

MRI (Magnetic Resonance Imaging) scans of the affected knee are taken within 12 weeks prior to surgery. MRI scans are taken as outlined below and are evaluated by the independent radiologist for determination of the effectiveness of the backfill and the presence of adverse events. Follow-up MRI scans are taken at the following intervals post-treatment: 1) Week 12 (+/−3 days) post-surgery; and 2) Week 24 (+/−3 days) post-surgery.

The subjects undergo a functional assessment by a designated assessor at the pre-treatment and 4, 12, and 24 week intervals. The subjects are evaluated at pre-treatment, 4 weeks, 12 weeks and at 24 weeks for clinical, MRI (12 and 24 weeks only), as well as complications and/or device related adverse events and concomitant medication usage.

The subjects are evaluated at surgery only for ICRS standard, for VAS at baseline level and 1, 3, and 6 months, for KOOS at baseline level and 1, 3, and 6 months, for Modified Cincinnati Rating System at baseline level and 1, 3, and 6 months.

Groups 3 receiving both the rhPDGF-BB and biphasic biocompatible matrix plug surgically implanted in an osteochondral defect located in a high-load-bearing region of the knee is reported to have accelerated time of healing at the defect site, as measured by MRI, ICRS, VAS, KOOS, Modified Cincinnati Rating System, and arthroscopy.

Unless defined otherwise, the meanings of all technical and scientific terms used herein are those commonly understood by one of skill in the art to which this invention belongs. It is to be understood that this invention is not limited to the particular methodology, protocols, and reagents described, as these may vary. One of skill in the art will also appreciate that any methods and materials similar or equivalent to those described herein can also be used to practice or test the invention.

The headings provided herein are not limitations of the various aspects or embodiments of the invention which can be had by reference to the specification as a whole.

1-27. (canceled)

28. A composition for treating an osteochondral defect comprising a biphasic biocompatible matrix and platelet derived growth factor (PDGF) in a solution,
wherein the biphasic biocompatible matrix comprises a dual-layer scaffolding material comprising:
a) a top layer, wherein the top layer comprises a cartilage phase
b) a bottom layer, wherein the bottom layer comprises an osseous phase,
and wherein the scaffolding material forms a porous structure.

29. The composition of claim 28, wherein the osseous phase comprises a calcium phosphate.

30. The composition of claim 28, wherein the osseous phase comprises a calcium phosphate and collagen.

31. The composition of claim 28, wherein the osseous phase comprises a calcium phosphate and an allograft material.

32. The composition of claim 28, wherein the osseous phase comprises a calcium phosphate, collagen and an allograft material.

33. The composition of claim 28, wherein the osseous phase comprises a collagen and an allograft material.

34. The composition of claim 28, wherein the cartilage phase comprises collagen.

35. The composition of claim 28, wherein the cartilage phase comprises glycosaminoglycan.

36. The composition of claim 28, wherein the cartilage phase comprises glycosaminoglycan and collagen.

37. The composition of claim 28, wherein the cartilage phase comprises glycosaminoglycan, an allograft material and collagen.

38. The composition of claim 28, wherein the biocompatible matrix further comprises a biocompatible binder.

39. A method for treating an osteochondral defect in an individual comprising administering to the individual an effective amount of a composition comprising a biphasic biocompatible matrix and platelet derived growth factor (PDGF) in a solution,
wherein the biphasic biocompatible matrix comprises a dual-layer scaffolding material comprising:
a) a top layer, wherein the top layer comprises a cartilage phase
b) a bottom layer, wherein the bottom layer comprises an osseous phase,
and wherein the scaffolding material forms a porous structure.

40. The method of claim 39, wherein the osteochondral defect is in a cartilage and a bone adjacent to the cartilage, and wherein the cartilage comprises articular cartilage.

41. The method of claim 39, wherein the osteochondral defect is in a cartilage and a bone adjacent to the cartilage, and wherein the bone adjacent to the cartilage comprises a subchondral bone or a cancellous bone.

42. The method of claim 39, wherein the at least one site of the osteochondral defect comprises the bone adjacent to the cartilage, the cartilage, an interface between the cartilage and the bone adjacent to the cartilage, or combinations thereof.

43. The method of claim 39, wherein the osseous phase comprises a calcium phosphate and collagen.

44. The method of claim 39, wherein the osseous phase comprises an allograft material and collagen.

45. The method of claim 39, wherein the cartilage phase comprises a glycosaminoglycan and collagen.

46. The method of claim 39, wherein the cartilage phase comprises a glycosaminoglycan and an allograft material.

47. The method of claim 39, wherein the biphasic biocompatible matrix further comprises a biocompatible binder.
